(19) United States
(12) Patent Application Publication
(10) Pub. No.: US 2005/0112121 A1
(43) Pub. Date: May 26, 2005

(54) Therapeutic and diagnostic methods and compositions based on notch proteins and nucleic acids

(75) Inventors: Spyridon Artavanis-Tsakonas, Hamden, CT (US); Richard Grant Fehon, Durham, NC (US); Panayiotis Zagouras, New Haven, CT (US); Christine Marie Blaumueller, New Haven, CT (US)

Correspondence Address: JONES DAY, 222 EAST 41ST ST, NEW YORK, NY, 10017, US

(73) Assignee: Yale University

(21) Appl. No.: 10/781,060

(22) Filed: Feb. 17, 2004

Related U.S. Application Data
(63) Continuation of application No. 09/564,504, filed on May 4, 2000, now abandoned.
Application No. 09/564,504 is a division of application No. 08/532,384, filed on Sep. 22, 1995, now Pat. No. 6,083,904.
Application No. 08/532,384 is a continuation of application No. 08/083,590, filed on Jun. 25, 1993, now Pat. No. 5,786,158.
Application No. 08/083,590 is a continuation-in-part of application No. 07/955,012, filed on Sep. 30, 1992, now abandoned.
Application No. 08/083,590 is a continuation-in-part of application No. 07/879,038, filed on Apr. 30, 1992, now abandoned.

(51) Int. Cl. A61K 39/395; A61K 38/17

(52) U.S. Cl. 424/144.1; 514/12

(57) ABSTRACT

The present invention relates to therapeutic and diagnostic methods and compositions based on Notch proteins and nucleic acids. The invention provides for treatment of disorders of cell fate or differentiation by administration of a therapeutic compound of the invention. Such therapeutic compounds (termed herein “Therapeutics”) include Notch proteins and analogs and derivatives (including fragments) thereof, antibodies thereto, nucleic acids encoding the Notch proteins, analogs, or derivatives, Notch antisense nucleic acids, as well as toporythmic proteins and derivatives which bind to or otherwise interact with Notch proteins and their encoding nucleic acids and antibodies. In a preferred embodiment, a Therapeutic of the invention is administered to treat a cancerous condition, or to prevent progression from a pre-neoplastic or non-malignant state into a neoplastic or a malignant state. In other embodiments, a Therapeutic is administered to treat a nervous system disorder or to promote tissue regeneration and repair. In one embodiment, Therapeutics which antagonize, or inhibit, Notch function (hereinafter “Antagonist Therapeutics”) are administered for therapeutic effect. In another embodiment, Therapeutics which promote Notch function (hereinafter “Agonist Therapeutics”) are administered for therapeutic effect. Diagnostic methods and methods of inhibiting Notch expression are also provided.

This application is a continuation-in-part of both copending application Ser. No. 07/955,012 filed Sep. 30, 1992, and copending application Ser. No. 07/879,038 filed Apr. 30, 1992, each of which is incorporated by reference herein in its entirety.

This invention was made in part with government support under grant numbers GM 19093 and NS 26084 awarded by the National Institutes of Health. The government has certain rights in the invention.

### 1. INTRODUCTION

The present invention relates to therapeutic compositions comprising Notch proteins, analogs and derivatives thereof, antibodies thereto, nucleic acids encoding the Notch proteins, derivatives or analogs, Notch antisense nucleic acids, and toporythmic proteins which bind to Notch and their nucleic acids and antibodies. Therapeutic and diagnostic methods are also provided.

### 2. BACKGROUND OF THE INVENTION

### 2.1. The Notch Gene and Protein

Null mutations in any one of the zygotic neurogenic loci—Notch (N), Delta (Dl), mastermind (mam), Enhancer of Split (E(spl), neuralized (neu), and big brain (bib)—result in hypertrophy of the nervous system at the expense of ventral and lateral epidermal structures. This effect is due to the misrouting of epidermal precursor cells into a neuronal pathway, and implies that neurogenic gene function is necessary to divert cells within the neurogenic region from a neuronal fate to an epithelial fate. Studies that assessed the effects of laser ablation of specific embryonic neuroblasts in grasshoppers (Doe and Goodman 1985, Dev. Biol. 111, 206-219) have shown that cellular interactions between neuroblasts and the surrounding accessory cells serve to inhibit these accessory cells from adopting a neuroblast fate. Together, these genetic and developmental observations have led to the hypothesis that the protein products of the neurogenic loci function as components of a cellular interaction mechanism necessary for proper epidermal development (Artavanis-Tsakonas, 1988, Trends Genet. 4, 95-100).

Sequence analyses (Wharton et al., 1985, Cell 43, 567-581; Kidd et al., 1986, Mol. Cell. Biol. 6, 3094-3108; Vassin et al., 1987, EMBO J. 6, 3431-3440; Kopczynski et al., 1988, Genes Dev. 2, 1723-1735) have shown that two of the neurogenic loci, Notch and Delta, appear to encode transmembrane proteins that span the membrane a single time. The Drosophila Notch gene encodes a ˜300 kd protein (we use “Notch” to denote this protein) with a large N-terminal extracellular domain that includes 36 epidermal growth factor (EGF)-like tandem repeats followed by three other cysteine-rich repeats, designated Notch/lin-12 repeats (Wharton et al., 1985, Cell 43, 567-581; Kidd et al., 1986, Mol. Cell Biol. 6, 3094-3108; Yochem et al., 1988, Nature 335, 547-550). The sequences of Xenopus (Coffman et al., 1990, Science 249:1438-1441) and a human Notch homolog termed TAN-1 (Ellisen et al., 1991, Cell 66:649-661) have also been reported. Delta encodes a ˜100 kd protein (we use “Delta” to denote DLZM, the protein product of the predominant zygotic and maternal transcripts; Kopczynski et al., 1988, Genes Dev. 2, 1723-1735) that has nine EGF-like repeats within its extracellular domain (Vassin et al., 1987, EMBO J. 6, 3431-3440; Kopczynski et al., 1988, Genes Dev. 2, 1723-1735). Although little is known about the functional significance of these repeats, the EGF-like motif has been found in a variety of proteins, including those involved in the blood clotting cascade (Furie and Furie, 1988, Cell 53, 505-518). In particular, this motif has been found in extracellular proteins such as the blood clotting factors 1× and X (Rees et al., 1988, EMBO J. 7, 2053-2061; Furie and Furie, 1988, Cell 53, 505-518), in other Drosophila genes (Knust et al., 1987, EMBO J. 761-766; Rothberg et al., 1988, Cell 55, 1047-1059), and in some cell-surface receptor proteins, such as thrombomodulin (Suzuki et al., 1987, EMBO J. 6, 1891-1897) and LDL receptor (Sudhof et al., 1985, Science 228, 815-822). A protein binding site has been mapped to the EGF repeat domain in thrombomodulin and urokinase (Kurosawa et al., 1988, J. Biol. Chem 263, 5993-5996; Appella et al., 1987, J. Biol. Chem. 262, 4437-4440).

An intriguing array of interactions between Notch and Delta mutations has been described (Vassin, et al., 1985, J. Neurogenet. 2, 291-308; Shepard et al., 1989, Genetics 122, 429438; Xu et al., 1990, Genes Dev., 4, 464-475). A number of genetic studies (summarized in Alton et al., 1989, Dev. Genet. 10, 261-272) has indicated that the gene dosages of Notch and Delta in relation to one another are crucial for normal development. A 50% reduction in the dose of Delta in a wild-type Notch background causes a broadening of the wing veins creating a “delta” at the base (Lindsley and Grell, 1968, Publication Number 627, Washington, D.C., Carnegie Institute of Washington). A similar phenotype is caused by a 50% increase in the dose of Notch in a wild-type Delta background (a “Confluens” phenotype; Welshons, 1965, Science 150, 1122-1129). This Delta phenotype is partially suppressed by a reduction in the Notch dosage. Work has shown that lethal interactions between alleles that correlate with alterations in the EGF-like repeats in Notch can be rescued by reducing the dose of Delta (Xu et al., 1990, Genes Dev. 4, 464-475). Xu et al. (1990, Genes Dev. 4, 464-475) found that null mutations at either Delta or mam suppress lethal interactions between heterozygous combinations of certain Notch alleles, known as the Abruptex (Ax) mutations. Ax alleles are associated with missense mutations within the EGF-like repeats of the Notch extracellular domain (Kelley et al., 1987, Cell 51, 539-548; Hartley et al., 1987, EMBO J. 6, 3407-3417).

Recent studies have shown that Notch and Delta, and Notch and Serrate, directly interact on'the molecular level (Fehon et al., 1990, Cell 61:523-534; Rebay et al., 1991, Cell 67:687-699).

Notch is expressed on axonal processes during the outgrowth of embryonic neurons (Johansen et al., 1989, J. Cell Biol. 109:2427-2440; Kidd et al., 1989, Genes Dev. 3:1113-1129; Fehon et al., 1991, J. Cell Biol. 113:657-669).

A study has shown that certain Ax alleles of Notch can severely alter axon pathfinding during sensory neural outgrowth in the imaginal discs, although it is not yet known whether aberrant Notch expression in the axon itself or the epithelium along which it grows is responsible for this defect (Palka et al., 1990, Development 109, 167-175).

### 2.2. Cancer

A neoplasm, or tumor, is a neoplastic mass resulting from abnormal uncontrolled cell growth, which may cause swelling on the body surface, and which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W. B. Saunders Co., Philadelphia, pp. 68-122).

Effective treatment and prevention of cancer remains a long-felt need, and a major goal of biomedical research.

### 3. SUMMARY OF THE INVENTION

The present invention relates to therapeutic and diagnostic methods and compositions based on Notch proteins and nucleic acids. The invention provides for treatment of disorders of cell fate or differentiation by administration of a therapeutic compound of the invention. Such therapeutic compounds (termed herein “Therapeutics”) include: Notch proteins and analogs and derivatives (including fragments) thereof; antibodies thereto; nucleic acids encoding the Notch proteins, analogs, or derivatives; Notch antisense nucleic acids; as well as toporythmic proteins and derivatives which bind to or otherwise interact with Notch proteins, and their encoding nucleic acids and antibodies. In a preferred embodiment, a Therapeutic of the invention is administered to treat a cancerous condition, or to prevent progression from a pre-neoplastic or non-malignant state into a neoplastic or a malignant state. In other specific embodiments, a Therapeutic of the invention is administered to treat a nervous system disorder or to promote tissue regeneration and repair.

In one embodiment, Therapeutics which antagonize, or inhibit, Notch function (hereinafter “Antagonist Therapeutics”) are administered for therapeutic effect; disorders which can be thus treated can be identified by in vitro assays such as described in Section 5.1, infra. Such Antagonist Therapeutics include but are not limited to Notch antisense nucleic acids, anti-Notch neutralizing antibodies, and competitive inhibitors of Notch protein-protein interactions (e.g., a protein comprising Notch ELR-11 and ELR-12 and derivatives thereof), all as detailed infra.

In another embodiment, Therapeutics which promote Notch function (hereinafter “Agonist Therapeutics”) are administered for therapeutic effect; disorders which can thus be treated can be identified by in vitro assays such as described in Section 5.1, infra. Such Agonist Therapeutics include but are not limited to Notch proteins and derivatives thereof comprising the intracellular domain, and proteins that interact with Notch (e.g., a protein comprising a Delta sequence homologous to Drosophila Delta amino acids 1-230 (see FIG. 1 and SEQ ID NO:2), or comprising a Serrate sequence homologous to Drosophila Serrate amino acids 79-282 (see FIG. 5 and SEQ ID NO:4)).

Disorders of cell fate, in particular hyperproliferative (e.g., cancer) or hypoproliferative disorders, involving aberrant or undesirable levels of expression or activity of Notch protein can be diagnosed by detecting such levels, as described more fully infra.

In a preferred aspect, a Therapeutic of the invention is a protein consisting of at least a fragment (termed herein “adhesive fragment”) of the proteins encoded by toporythmic genes which mediates binding to Notch proteins or adhesive fragments thereof. Toporythmic genes, as used herein, shall mean the genes Notch, Delta, and Serrate, as well as other members of the Delta/Serrate family which may be identified by virtue of sequence homology or genetic interaction, and in general, members of the “Notch cascade” or the “Notch group” of genes, which are identified by molecular interactions (e.g., binding in vitro) or genetic interactions (as detected phenotypically, e.g., in Drosophila).

In another aspect, the invention is directed to human Notch proteins; in particular, that encoded by the hN homolog, and proteins comprising the extracellular domain of the protein and subsequences thereof. Nucleic acids encoding the foregoing, and recombinant cells are also provided.

### 3.1. Definitions

As used herein, the following terms shall have the meanings indicated:

| | |
|---|---|
| AA = | amino acid |
| EGF = | epidermal growth factor |
| ELR = | EGF-like (homologous) repeat |
| IC = | intracellular |
| PCR = | polymerase chain reaction |

As used herein, underscoring the name of a gene shall indicate the gene, in contrast to its encoded protein product which is indicated by the name of the gene in the absence of any underscoring. For example, “Notch” shall mean the Notch gene, whereas “Notch” shall indicate the protein product of the Notch gene.

### 4. DESCRIPTION OF THE FIGURES

FIG. 1. Primary Nucleotide Sequence of the Delta cDNA D11 (SEQ ID NO:1) and Delta amino acid sequence (SEQ ID NO:2). The DNA sequence of the 5′-3′ strand of the D11 cDNA is shown, which contains a number of corrections in comparison to that presented in Kopczynkski et al. (1988, Genes Dev. 2:1723-1735).

FIG. 2. Notch Expression Constructs and the Deletion Mapping of the Delta/Serrate Binding Domain. S2 cells in log phase growth were transiently transfected with the series of expression constructs shown; the drawings represent the predicted protein products of the various Notch deletion mutants created. All expression constructs were derived from construct #1 pMtNMg. Transiently transfected cells were mixed with Delta expressing cells from the stably transformed line L49-6-7 or with transiently transfected Serrate expressing cells, induced with CuSO₄, incubated under aggregation conditions and then scored for their ability to aggregate using specific antisera and immunofluorescence microscopy. Aggregates were defined as clusters of four or more cells containing both Notch and Delta/Serrate expressing cells. The values given for % Aggregation refer to the percentage of all Notch expressing cells found in such clusters either with Delta (Dl) (left column) or with Serrate (Ser) (right column). The various Notch deletion constructs are represented diagrammatically with splice lines indicating the ligation junctions. Each EGF repeat is denoted as a stippled rectangular box and numbers of the EGF repeats on either side of a ligation junction are noted. At the ligation junctions, partial EGF repeats produced by the various deletions are denoted by open boxes and closed brackets (for example see #23 ΔCla+EGF(10-12)). Constructs #3-13 represent the ClaI deletion series. As diagrammed, four of the ClaI sites, in repeats 7, 9, 17 and 26, break the repeat in the middle, immediately after the third cysteine (denoted by open box repeats; see FIG. 3 for further clarification), while the fifth and most 3′ site breaks neatly between EGF repeats 30 and 31 (denoted by closed box repeat 31; again see FIG. 3). In construct #15 split, EGF repeat 14 which carries the split point mutation, is drawn as a striped box. In construct #33 ΔCla+XEGF(10-13), the Xenopus Notch derived EGF repeats are distinguished from Drosophila repeats by a different pattern of shading. SP, signal peptide; EGF, epidermal growth factor repeat; N, Notch/lin-12 repeat; TM, transmembrane domain; cdc10, cdc10/ankyrin repeats; PA, putative nucleotide binding consensus sequence; opa, polyglutamine stretch termed opa; Dl, Delta; Ser, Serrate.

FIG. 3. Detailed Structure of Notch Deletion Constructs #19-24: Both EGF Repeats 11 and 12 are Required for Notch-Delta Aggregation. EGF repeats 10-13 are diagrammed at the top showing the regular spacing of the six cysteine residues (C). PCR products generated for these constructs (names and numbers as given in FIG. 2) are represented by the heavy black lines and the exact endpoints are noted relative to the various EGF repeats. Ability to aggregate with Delta is recorded as (+) or (−) for each construct. The PCR fragments either break the EGF repeats in the middle, just after the third cysteine in the same place as four out of the five ClaI sites, or exactly in between two repeats in the same place as the most C-terminal ClaI site.

FIG. 4. Comparison of Amino Acid Sequence of EGF Repeats 11 and 12 from Drosophila and Xenopus Notch. The amino acid sequence of EGF repeats 11 and 12 of Drosophila Notch (SEQ ID NO:14) (Wharton et al., 1985, Cell 43:567-581; Kidd et al., 1986, Mol. Cell Biol. 6:3094-3108) is aligned with that of the same two EGF repeats from Xenopus Notch (SEQ ID NO:15) (Coffman et al., 1990, Science 249:1438-1441). Identical amino acids are boxed. The six conserved cysteine residues of each EGF repeat and the Ca⁺⁺ binding consensus residues (Rees et al., 1988, EMBO J. 7:2053-2061) are marked with an asterisk (*). The leucine to proline change found in the Xenopus PCR clone that failed to aggregate is noted underneath.

FIG. 5. Nucleic Acid Sequence Homologies Between Serrate and Delta. A portion of the Drosophila Serrate nucleotide sequence (SEQ ID NO:3), with the encoded Serrate protein sequence (SEQ ID NO:4) written below (Fleming et al., 1990, Genes & Dev. 4:2188-2201 at 2193-94) is shown. The four regions showing high sequence homology with the Drosophila Delta sequence are numbered above the line and indicated by brackets. The total region of homology spans nucleotide numbers 627 through 1290 of the Serrate nucleotide sequence (numbering as in FIG. 4 of Fleming et al., 1990, Genes & Dev. 4:2188-2201).

FIG. 6. Schematic Diagram of Human Notch Clones. A schematic diagram of human Notch is shown. Heavy bold-face lines below the diagram show that portion of the Notch sequence contained in each of the four cDNA clones. The location of the primers used in PCR, and their orientation, are indicated by arrows.

FIG. 7. Human Notch Sequences Aligned with Drosophila Notch Sequence. Numbered vertical lines correspond to Drosophila Notch coordinates. Horizontal lines below each map show where clones lie relative to stretches of sequence (thick horizontal lines).

FIG. 8. Nucleotide Sequences of Human Notch Contained in Plasmid cDNA Clone hN2k. FIG. 8A: The DNA sequence (SEQ ID NO:5) of a portion of the human Notch insert is shown, starting at the EcoRI site at the 3′ end, and proceeding in the 3′ to 5′ direction. FIG. 8B: The DNA sequence (SEQ ID NO:6) of a portion of the human Notch insert is shown, starting at the EcoRI site at the 5′ end, and proceeding in the 5′ to 3′ direction. FIG. 8C: The DNA sequence (SEQ ID NO:7) of a portion of the human Notch insert is shown, starting 3′ of the sequence shown in FIG. 8B, and proceeding in the 5′ to 3′ direction. The sequences shown are tentative, subject to confirmation by determination of overlapping sequences.

FIG. 9. Nucleotide Sequences of Human Notch Contained in Plasmid cDNA clone hN4k. FIG. 9A: The DNA sequence (SEQ ID NO:8) of a portion of the human Notch insert is shown, starting at the EcoRI site at the 5′ end, and proceeding in the 5′ to 3′ direction. FIG. 9B: The DNA sequence (SEQ ID NO:9) of a portion of the human Notch insert is shown, starting near the 3′ end, and proceeding in the 3′ to 5′ direction. The sequences shown are tentative, subject to confirmation by determination of overlapping sequences.

FIG. 10. DNA (SEQ ID NO:10) and Amino Acid (SEQ ID NO:11) Sequences of Human Notch Contained in Plasmid cDNA Clone hN3k.

FIG. 11. DNA (SEQ ID NO:12) and Amino Acid (SEQ ID NO:13) Sequences of Human Notch Contained in Plasmid cDNA Clone hN5k.

FIG. 12. Comparison of hN5k With Other Notch Homologs. FIG. 12A. Schematic representation of Drosophila Notch. Indicated are the signal sequence (signal), the 36 EGF-like repeats, the three Notch/lin-12 repeats, the transmembrane domain (TM), the six CDC10 repeats, the OPA repeat, and the PEST (proline, glutamic acid, serine, threonine)-rich region. FIG. 12B. Alignment of the deduced amino acid sequence of hNSk with sequences of other Notch homologs. Amino acids are numbered on the left side. The cdc10 and PEST-rich regions are both boxed, and individual cdc10 repeats are marked. Amino acids which are identical in three or more sequences are highlighted. The primers used to clone hN5k are indicated below the sequences from which they were designed. The nuclear localization sequence (NLS), casein kinase II (CKII), and cdc2 kinase (cdc2) sites of the putative CcN motif of the vertebrate Notch homologs are boxed. The possible bipartite nuclear targeting sequence (BNTS) and proximal phosphorylation sites of Drosophila Notch are also boxed.

FIG. 13. Aligned amino acid sequences of Notch proteins of various species. humN: the human Notch protein encoded by the hN homolog (contained in part in plasmid hN5k) (SEQ ID NO:19). TAN-1: the human Notch protein encoded by the TAN-1 homolog (SEQ ID NO:20) (the sequence shown is derived partly from our own work and partly from the TAN-1 sequence as published by Ellisen et al., 1991, Cell 66:649-661); Xen N: Xenopus Notch protein (Coffman et al., 1990, Science 249:1438-1441). Dros N: Drosophila Notch protein (Wharton et al., 1985, Cell 43:567-581). Structural domains are indicated.

FIG. 14. Immunocytochemical staining of breast cancer tissue from a human patient. Malignant breast tissue in a sample obtained from a human patient was embedded in a paraffin section, and subjected to immunocytochemical staining with anti-human Notch monoclonal antibody P4, directed against the TAN-1 protein. Non-malignant breast tissue exhibited much less staining (not shown).

FIG. 15. Immunocytochemical staining of colon tissue from a human patient with colon cancer. A colon tissue sample obtained from a patient with colon cancer was embedded in a paraffin section, and subjected to immunocytochemical staining with anti-human Notch monoclonal antibody P1, directed against the hN-encoded protein. Areas of increased staining are those areas in which malignant cells are present, as determined by cell morphology.

FIG. 16. Immunocytochemical staining of cervical tissue. Human tissue samples were obtained, containing cancer of the cervix (FIG. 16A) or normal cervical epithelium (FIG. 16B) from the same patient, embedded in a paraffin section, and subjected to immunocytochemical staining with anti-human Notch monoclonal antibody directed against the TAN-1 protein. Areas containing malignant cells (as determined by morphology) exhibited increasing staining relative to non-malignant cells. Among non-malignant cells, connective tissue and the basal layer of the epithelium (containing stem cells) stained with the anti-Notch antibody.

FIG. 17. DNA (SEQ ID NO:21) and encoded amino acid sequence (contained in SEQ ID NO:19) of human Notch homolog hN. The entire DNA coding sequence is presented (as well as noncoding sequence), with the exclusion of that encoding the initiator Met.

### 5. DETAILED DESCRIPTION OF THE INVENTION

The present invention relates to therapeutic and diagnostic methods and compositions based on Notch proteins and nucleic acids. The invention provides for treatment of disorders of cell fate or differentiation by administration of a therapeutic compound of the invention. Such therapeutic compounds (termed herein “Therapeutics”) include: Notch proteins and analogs and derivatives (including fragments) thereof; antibodies thereto; nucleic acids encoding the Notch proteins, analogs, or derivatives; Notch antisense nucleic acids; as well as toporythmic proteins and derivatives and analogs thereof which bind to or otherwise interact with Notch proteins, and their encoding nucleic acids and antibodies. Also included are proteins and derivatives and analogs thereof which are capable of inhibiting the interactions of a Notch protein with another toporythmic protein (e.g. Delta, Serrate). In a preferred embodiment, a Therapeutic of the invention is administered to treat a cancerous condition, or to prevent progression from a pre-neoplastic or non-malignant state (e.g., metaplastic condition) into a neoplastic or a malignant state. In another specific embodiment, a Therapeutic of the invention is administered to treat a nervous system disorder, such as nerve injury or a degenerative disease. In yet another specific embodiment, a Therapeutic of the invention is administered to promote tissue regeneration and repair for treatment of various conditions.

In one embodiment, Therapeutics which antagonize, or inhibit, Notch function (hereinafter “Antagonist Therapeutics”) are administered for therapeutic effect; disorders which can be thus treated can be identified by in vitro assays such as described in Section 5.1, infra. Such Antagonist Therapeutics include but are not limited to Notch antisense nucleic acids, anti-Notch neutralizing antibodies, competitive inhibitors of Notch protein-protein interactions (e.g., a protein comprising Notch ELR-11 and ELR-12), and molecules which interfere with notch intracellular function such as that mediated by the cdc10 repeats, as detailed infra.

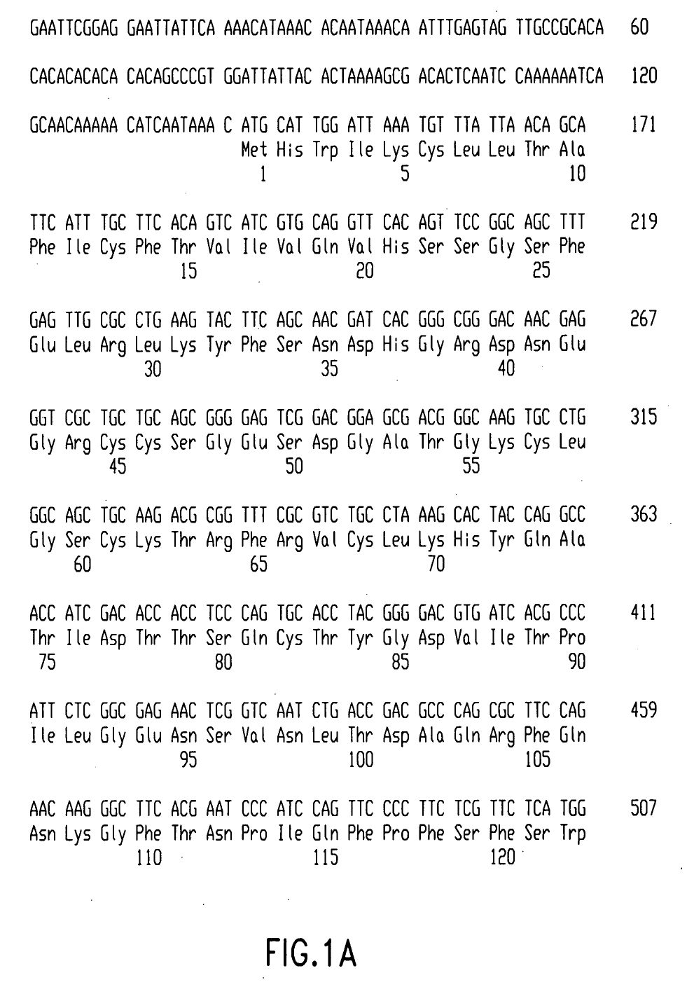

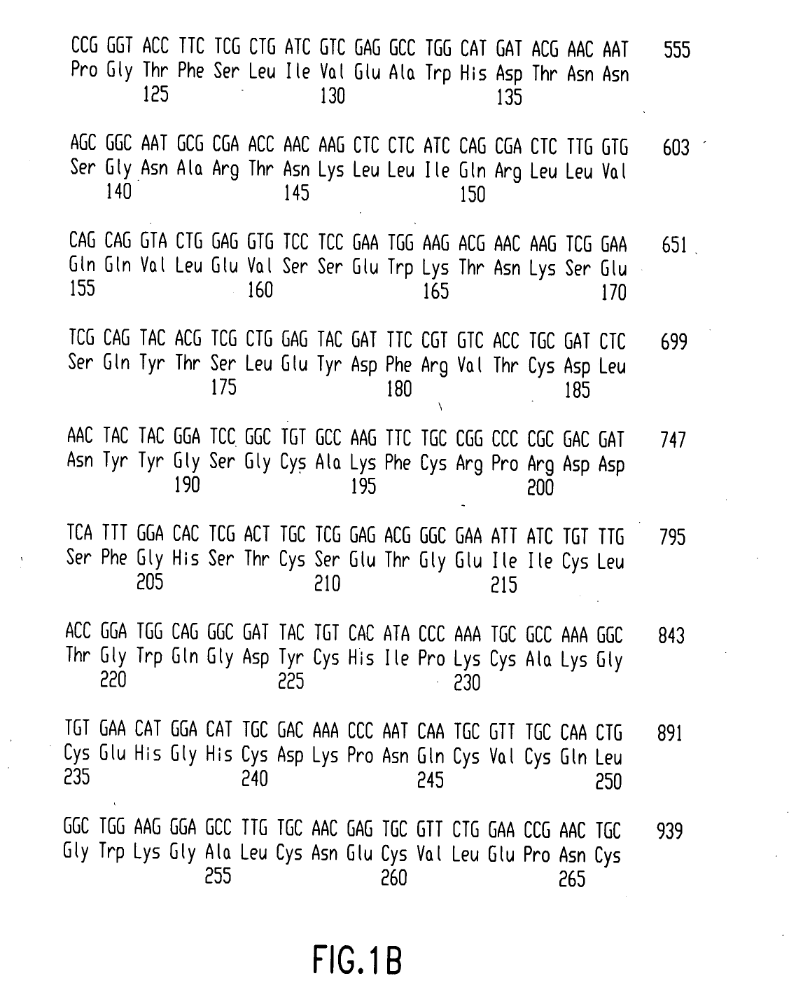

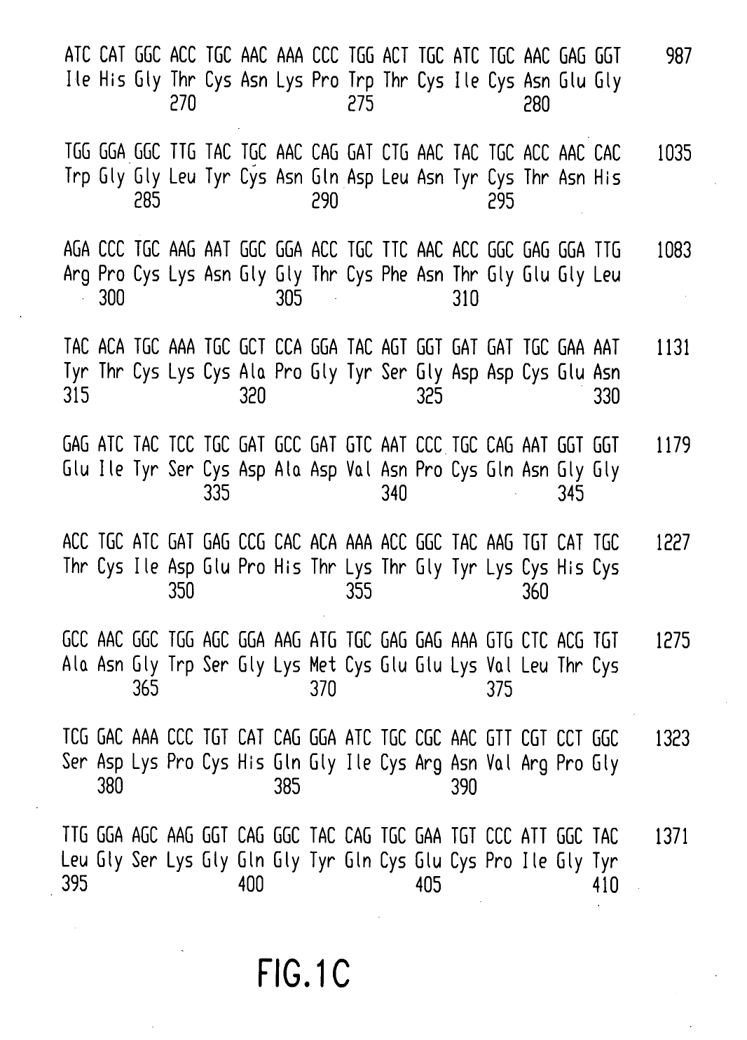

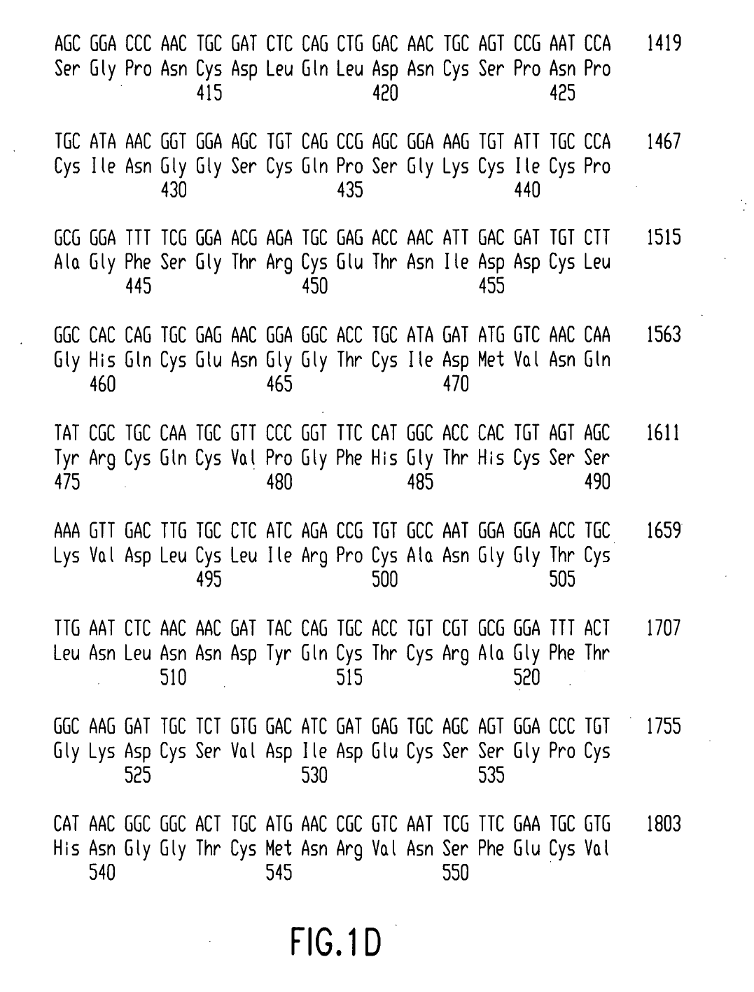

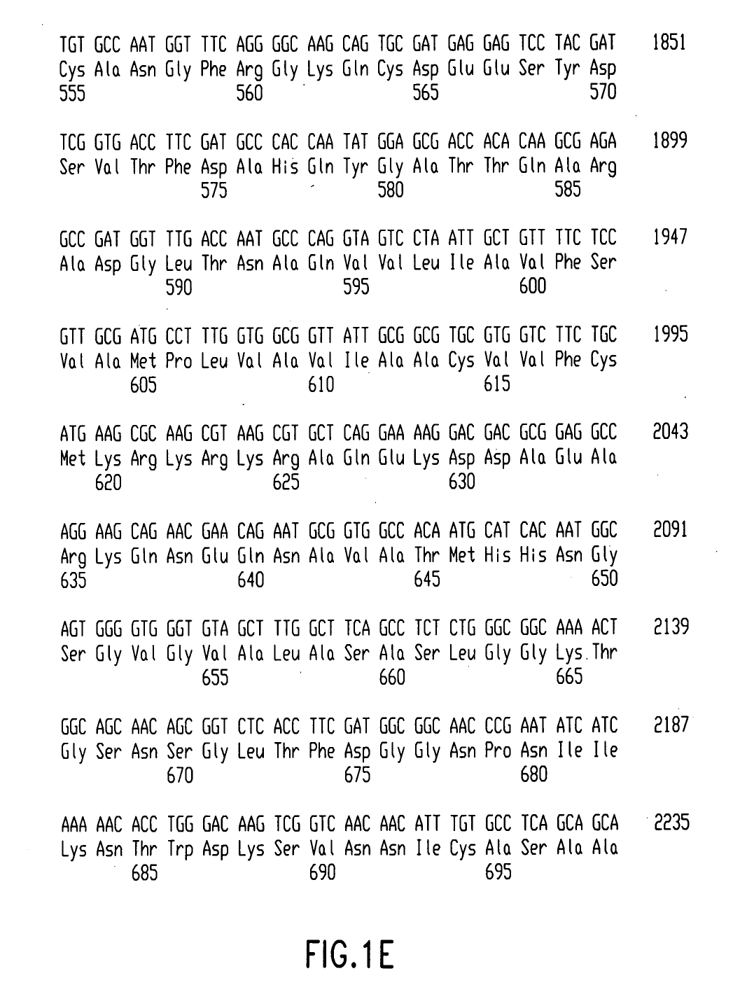

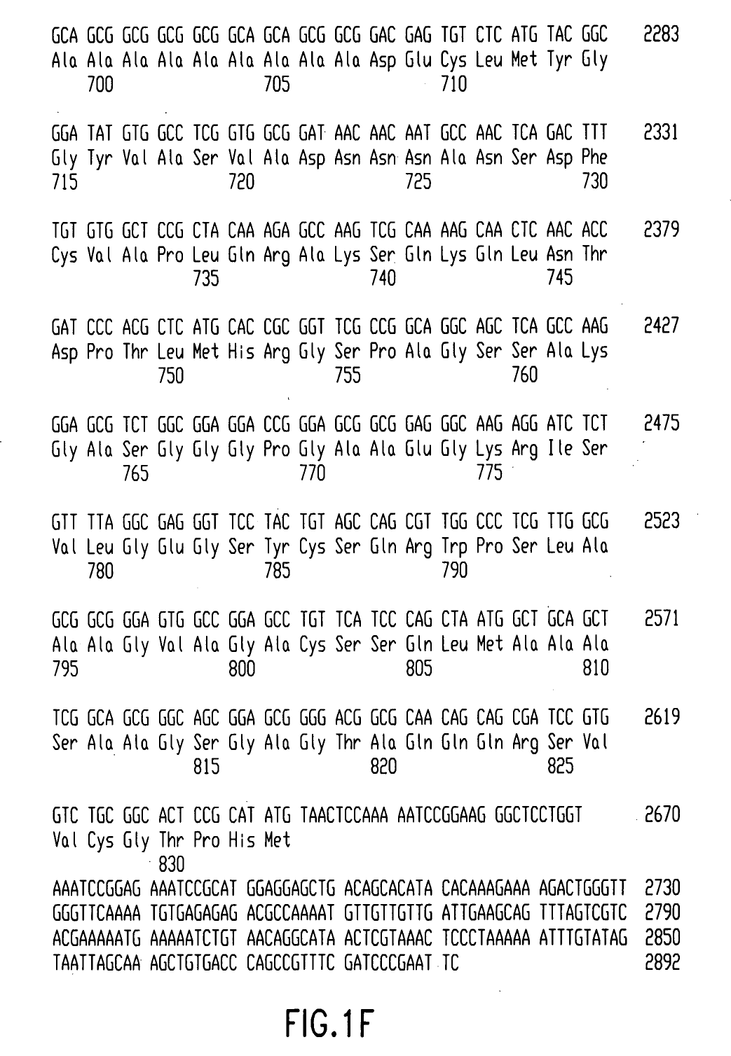

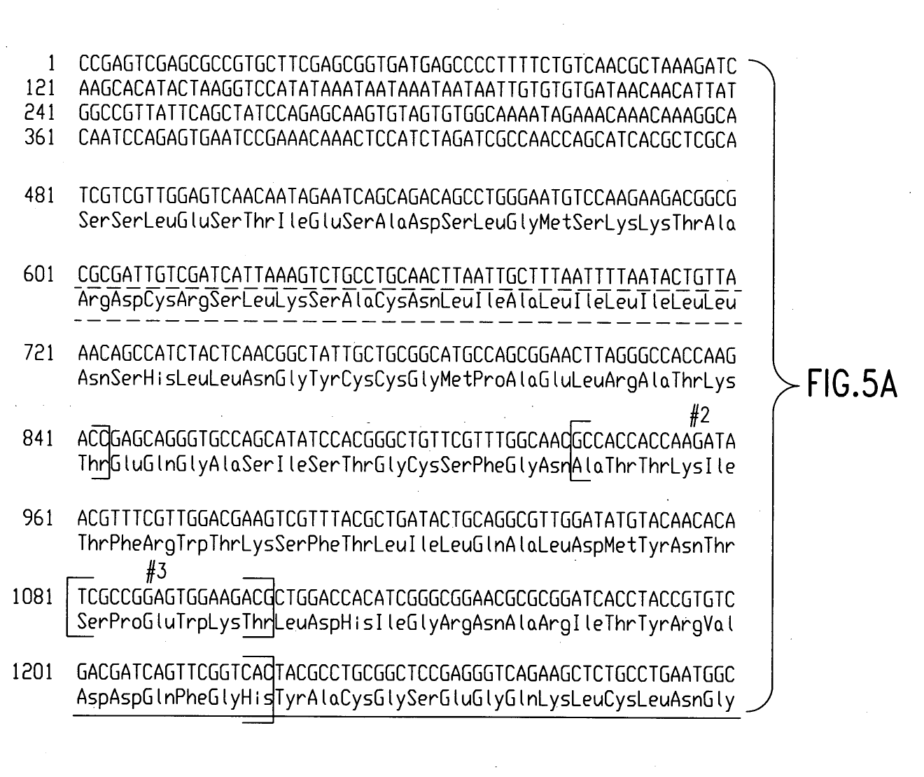

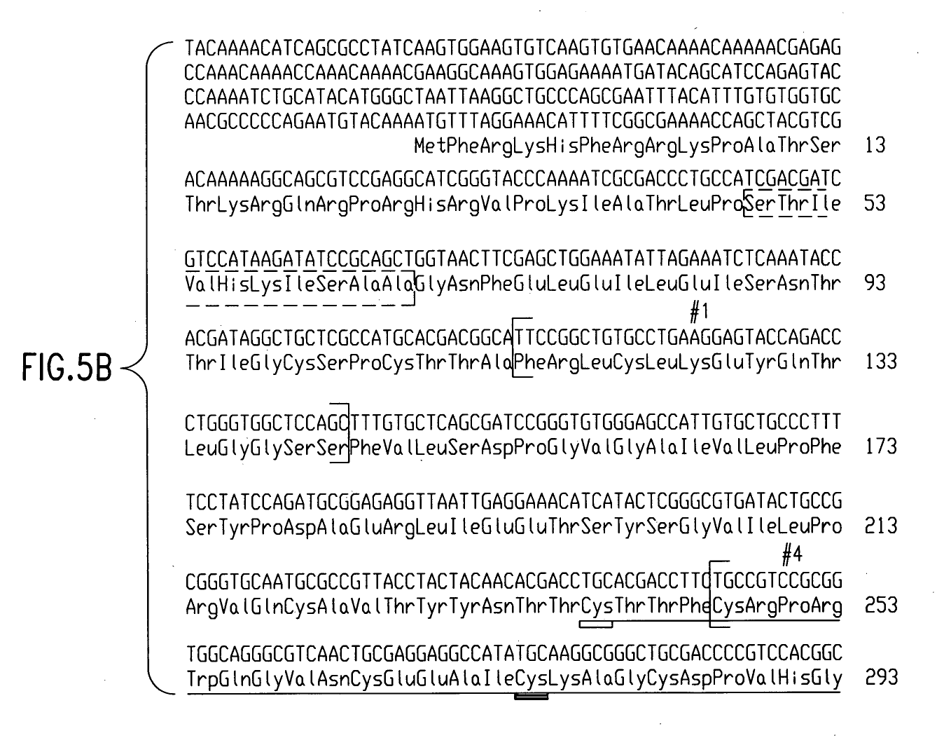

In another embodiment, Therapeutics which promote Notch function (hereinafter “Agonist Therapeutics”) are administered for therapeutic effect; disorders which can thus be treated can be identified by in vitro assays such as described in Section 5.1, infra. Such Agonist Therapeutics include but are not limited to Notch proteins and derivatives thereof comprising the intracellular domain, Notch nucleic acids encoding the foregoing, and proteins comprising toporythmic protein domains that interact with Notch (e.g., a protein comprising an extracellular domain of a Delta protein or a Delta sequence homologous to Drosophila Delta amino acids 1-230 (see FIG. 1 and SEQ ID NO:2), or comprising a Serrate sequence homologous to Drosophila Serrate amino acids 79-282 (see FIG. 5 and SEQ ID NO:4)).

Disorders of cell fate, in particular precancerous conditions such as metaplasia and dysplasia, and hyperproliferative (e.g., cancer) or hypoproliferative disorders, involving aberrant or undesirable levels of expression or activity of Notch protein can be diagnosed by detecting such levels, as described more fully infra.

In a preferred aspect, a Therapeutic of the invention is a protein consisting of at least a fragment (termed herein “adhesive fragment”) of the proteins encoded by toporythmic genes which mediates binding to Notch proteins or adhesive fragments thereof. Toporythmic genes, as used herein, shall mean the genes Notch, Delta, and Serrate, as well as other members of the Delta/Serrate family which may be identified by virtue of sequence homology or genetic interaction, and, more generally, members of the “Notch cascade” or the “Notch group” of genes, which are identified by molecular interactions (e.g., binding in vitro) or genetic interactions (as detected phenotypically, e.g., in Drosophila).

For clarity of disclosure, and not by way of limitation, the detailed description of the invention is divided into the following subsections:
(i) Therapeutic Uses;
(ii) Prophylactic Uses;
(iii) Demonstration of Therapeutic or Prophylactic Utility;
(iv) Therapeutic/Prophylactic Administration and Compositions;
(v) Antisense Regulation of Notch Expression;
(vi) Diagnostic Utility;
(vii) Notch Nucleic Acids;
(viii) Recombinant Production of Protein Therapeutics;
(ix) Derivatives and Analogs of Notch and Other Toporythmic Proteins;
(x) Assays of Notch Proteins, Derivatives and Analogs; and
(xi) Antibodies to Notch Proteins, Derivatives and Analogs.

### 5.1. Therapeutic Uses

As stated supra, the Antagonist Therapeutics of the invention are those Therapeutics which antagonize, or inhibit, a Notch function. Such Antagonist Therapeutics are most preferably identified by use of known convenient in vitro assays, e.g., based on their ability to inhibit binding of Notch to other proteins (see Sections 6-8 herein), or inhibit any known Notch function as assayed in vitro, although genetic assays (e.g., in Drosophila) may also be employed. In a preferred embodiment, the Antagonist Therapeutic is a protein or derivative thereof comprising a functionally active fragment such as an adhesive fragment of Notch. In specific embodiments, such an Antagonist Therapeutic may be those adhesive proteins encoded by the appropriate constructs described in Sections 6 and 7 infra, or proteins comprising the Notch extracellular region, in particular ELR-11 and ELR-12, or an antibody thereto, or an analog/competitive inhibitor of a Notch intracellular signal-transducing region, a nucleic acid capable of expressing a Notch adhesive fragment, or a Notch antisense nucleic acid (see Section 5.5 herein). It should be noted that in certain instances, a Notch adhesive fragment (or possibly other presumed Antagonist Therapeutics) may alternatively act as an Agonist Therapeutic, depending on the developmental history of the tissue being exposed to the Therapeutic; preferably, suitable in vitro or in vivo assays, as described infra, should be utilized to determine the effect of a specific Therapeutic and whether its administration is indicated for treatment of the affected tissue.

In another embodiment of the invention, a nucleic acid containing a portion of a Notch gene is used, as an Antagonist Therapeutic, to promote Notch inactivation by homologous recombination (Koller and Smithies, 1989, Proc. Natl. Acad. Sci. USA 86:8932-8935; Zijlstra et al., 1989, Nature 342:435-438).

The Agonist Therapeutics of the invention, as described supra, promote Notch function. Such Agonist Therapeutics include but are not limited to proteins and derivatives comprising the portions of toporythmic proteins such as Delta or Serrate that mediate binding to Notch, and nucleic acids encoding the foregoing (which can be administered to express their encoded products in vivo). In a specific embodiment, such a portion of Delta is D. melanogaster Delta amino acids 1-230 (SEQ ID NO:1) or a portion of a human Delta most homologous thereto. In another specific embodiment, such a portion of Serrate is D. melanogaster Serrate amino acids 79-282 (SEQ ID NO:5), or a portion of a human Serrate most homologous thereto. In other specific embodiments, such a portion of Delta or Serrate is the extracellular portion of such protein.

Further descriptions and sources of Therapeutics of the inventions are found in Sections 5.4 through 5.8 herein.

The Agonist and Antagonist Therapeutics of the invention have therapeutic utility for disorders of cell fate. The Agonist Therapeutics are administered therapeutically (including prophylactically): (1) in diseases or disorders involving an absence or decreased (relative to normal, or desired) levels of Notch function, for example, in patients where Notch protein is lacking, genetically defective, biologically inactive or underactive, or underexpressed; and (2) in diseases or disorders wherein in vitro (or in vivo) assays (see infra) indicate the utility of Notch agonist administration. The absence or decreased levels in Notch function can be readily detected, e.g., by obtaining a patient tissue sample (e.g., from biopsy tissue) and assaying it in vitro for protein levels, structure and/or activity of the expressed Notch protein. Many methods standard in the art can be thus employed, including but not limited to immunoassays to detect and/or visualize Notch protein (e.g., Western blot, immunoprecipitation followed by sodium dodecyl sulfate polyacrylamide gel electrophoresis, immunocytochemistry, etc.; see also those assays listed in Section 5.6, infra), and/or hybridization assays to detect Notch expression by detecting and/or visualizing Notch mRNA (e.g., Northern assays, dot blots, in situ hybridization, etc.)

In vitro assays which can be used to determine whether administration of a specific Agonist Therapeutic or Antagonist Therapeutic is indicated, include in vitro cell culture assays in which a patient tissue sample is grown in culture, and exposed to or otherwise administered a Therapeutic, and the effect of such Therapeutic upon the tissue sample is observed. In one embodiment, where the patient has a malignancy, a sample of cells from such malignancy is plated out or grown in culture, and the cells are then exposed to a Therapeutic. A Therapeutic which inhibits survival or growth of the malignant cells (e.g., by promoting terminal differentiation) is selected for therapeutic use in vivo. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc. In a specific aspect, the malignant cell cultures are separately exposed to (1) an Agonist Therapeutic, and (2) an Antagonist Therapeutic; the result of the assay can indicate which type of Therapeutic has therapeutic efficacy.

In another embodiment, a Therapeutic is indicated for use which exhibits the desired effect, inhibition or promotion of cell growth, upon a patient cell sample from tissue having or suspected of having a hyper- or hypoproliferative disorder, respectively. Such hyper- or hypoproliferative disorders include but are not limited to those described in Sections 5.1.1 through 5.1.3 infra.

In another specific embodiment, a Therapeutic is indicated for use in treating nerve injury or a nervous system degenerative disorder (see Section 5.1.2) which exhibits in vitro promotion of nerve regeneration/neurite extension from nerve cells of the affected patient type.

In addition, administration of an Antagonist Therapeutic of the invention is also indicated in diseases or disorders determined or known to involve a Notch dominant activated phenotype (“gain of function” mutations.) Administration of an Agonist Therapeutic is indicated in diseases or disorders determined or known to involve a Notch dominant negative phenotype (“loss of function” mutations). We have investigated the functions of various structural domains of the Notch protein in vivo, by ectopically expressing a series of Drosophila Notch deletion mutants under the hsp70 heat-shock promoter, as well as eye-specific promoters. Two classes of dominant phenotypes were observed, one suggestive of Notch loss of function mutations and the other of Notch gain-of-function mutations. Dominant “activated” phenotypes resulted from overexpression of a protein lacking most extracellular sequences, while dominant “negative” phenotypes resulted from overexpression of a protein lacking most intracellular sequences. Our results indicate that Notch functions as a receptor whose extracellular domain mediates ligand-binding, resulting in the transmission of developmental signals by the cytoplasmic domain. The phenotypes observed also suggested that the cdc10/ankyrin repeat region within the intracellular domain plays an essential role in Notch mediated signal transduction events (intracellular function).

In various specific embodiments, in vitro assays can be carried out with representative cells of cell types involved in a patient's disorder, to determine if a Therapeutic has a desired effect upon such cell types.

In another embodiment, cells of a patient tissue sample suspected of being pre-neoplastic are similarly plated out or grown in vitro, and exposed to a Therapeutic. The Therapeutic which results in a cell phenotype that is more normal (i.e., less representative of a pre-neoplastic state, neoplastic state, malignant state, or transformed phenotype) is selected for therapeutic use. Many assays standard in the art can be used to assess whether a pre-neoplastic state, neoplastic state, or a transformed or malignant phenotype, is present (see Section 5.2.1). For example, characteristics associated with a transformed phenotype (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250,000 dalton surface protein, etc. (see Luria et al., 1978, General Virology, 3d Ed., John Wiley & Sons, New York pp. 436-446).

In other specific embodiments, the in vitro assays described supra can be carried out using a cell line, rather than a cell sample derived from the specific patient to be treated, in which the cell line is derived from or displays characteristic(s) associated with the malignant, neoplastic or pre-neoplastic disorder desired to be treated or prevented, or is derived from the neural or other cell type upon which an effect is desired, according to the present invention.

The Antagonist Therapeutics are administered therapeutically (including prophylactically): (1) in diseases or disorders involving increased (relative to normal, or desired) levels of Notch function, for example, where the Notch protein is overexpressed or overactive; and (2) in diseases or disorders wherein in vitro (or in vivo) assays indicate the utility of Notch antagonist administration. The increased levels of Notch function can be readily detected by methods such as those described above, by quantifying protein and/or RNA. In vitro assays with cells of patient tissue sample or the appropriate cell line or cell type, to determine therapeutic utility, can be carried out as described above.

### 5.1.1. Malignancies

Malignant and pre-neoplastic conditions which can be tested as described supra for efficacy of intervention with Antagonist or Agonist Therapeutics, and which can be treated upon thus observing an indication of therapeutic utility, include but are not limited to those described below in Sections 5.1.1 and 5.2.1.

Malignancies and related disorders, cells of which type can be tested in vitro (and/or in vivo), and upon observing the appropriate assay result, treated according to the present invention, include but are not limited to those listed in Table 1 (for a review of such disorders, see Fishman et al., 1985, Medicine, 2d Ed., J. B. Lippincott Co., Philadelphia):

TABLE 1
MALIGNANCIES AND RELATED DISORDERS

```
Leukemia
      acute leukemia
            acute lymphocytic leukemia
            acute myelocytic leukemia
                  myeloblastic
                  promyelocytic
                  myelomonocytic
                  monocytic
                  erythroleukemia
      chronic leukemia
            chronic myelocytic (granulocytic) leukemia
            chronic lymphocytic leukemia
Polycythemia vera
Lymphoma
      Hodgkin's disease
      non-Hodgkin's disease
Multiple myeloma
Waldenström's macroglobulinemia
Heavy chain disease
Solid tumors
      sarcomas and carcinomas
            fibrosarcoma
            myxosarcoma
            liposarcoma
            chondrosarcoma
            osteogenic sarcoma
            chordoma
            angiosarcoma
            endotheliosarcoma
            lymphangiosarcoma
            lymphangioendotheliosarcoma
            synovioma
            mesothelioma
            Ewing's tumor
            leiomyosarcoma
            rhabdomyosarcoma
            colon carcinoma
            pancreatic cancer
            breast cancer
            ovarian cancer
            prostate cancer
            squamous cell carcinoma
            basal cell carcinoma
            adenocarcinoma
            sweat gland carcinoma
            sebaceous gland carcinoma
            papillary carcinoma
            papillary adenocarcinomas
            cystadenocarcinoma
            medullary carcinoma
            bronchogenic carcinoma
            renal cell carcinoma
            hepatoma
            bile duct carcinoma
            choriocarcinoma
            seminoma
            embryonal carcinoma
            Wilms' tumor
            cervical cancer
            testicular tumor
            lung carcinoma
            small cell lung carcinoma
            bladder carcinoma
            epithelial carcinoma
            glioma
            astrocytoma
            medulloblastoma
            craniopharyngioma
            ependymoma
            pinealoma
            hemangioblastoma
            acoustic neuroma
            oligodendroglioma
            menangioma
            melanoma
            neuroblastoma
            retinoblastoma
```

In specific embodiments, malignancy or dysproliferative changes (such as metaplasias and dysplasias) are treated or prevented in epithelial tissues such as those in the cervix, esophagus, and lung.

As detailed in the examples section 10.1 infra, malignancies of the breast, colon, and cervix exhibit increased expression of human Notch relative to such non-malignant tissue. Thus, in specific embodiments, malignancies of the breast, colon, or cervix are treated or prevented by administering an effective amount of an Antagonist Therapeutic of the invention. The presence of increased Notch expression in breast, colon, and cervical cancer suggests that many more cancerous conditions exhibit upregulated Notch. Thus, we envision that many more cancers, e.g., seminoma, melanoma, and lung cancer, can be treated or prevented by administration of an Antagonist Therapeutic.

### 5.1.2. Nervous System Disorders

Nervous system disorders, involving cell types which can be tested as described supra for efficacy of intervention with Antagonist or Agonist Therapeutics, and which can be treated upon thus observing an indication of therapeutic utility, include but are not limited to nervous system injuries, and diseases or disorders which result in either a disconnection of axons, a diminution or degeneration of neurons, or demyelination. Nervous system lesions which may be treated in a patient (including human and non-human mammalian patients) according to the invention include but are not limited to the following lesions of either the central (including spinal cord, brain) or peripheral nervous systems:
(i) traumatic lesions, including lesions caused by physical injury or associated with surgery, for example, lesions which sever a portion of the nervous system, or compression injuries;
(ii) ischemic lesions, in which a lack of oxygen in a portion of the nervous system results in neuronal injury or death, including cerebral infarction or ischemia, or spinal cord infarction or ischemia;
(iii) malignant lesions, in which a portion of the nervous system is destroyed or injured by malignant tissue which is either a nervous system associated malignancy or a malignancy derived from non-nervous system tissue;
(iv) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis;
(v) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis;
(vi) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration;
(vii) neurological lesions associated with systemic diseases including but not limited to diabetes (diabetic neuropathy, Bell's palsy), systemic lupus erythematosus, carcinoma, or sarcoidosis;
(viii) lesions caused by toxic substances including alcohol, lead, or particular neurotoxins; and
(ix) demyelinated lesions in which a portion of the nervous system is destroyed or injured by a demyelinating disease including but not limited to multiple sclerosis, human immunodeficiency virus-associated myelopathy, transverse myelopathy or various etiologies, progressive multifocal leukoencephalopathy, and central pontine myelinolysis.

Therapeutics which are useful according to the invention for treatment of a nervous system disorder may be selected by testing for biological activity in promoting the survival or differentiation of neurons (see also Section 5.1). For example, and not by way of limitation, Therapeutics which elicit any of the following effects may be useful according to the invention:
(i) increased survival time of neurons in culture;
(ii) increased sprouting of neurons in culture or in vivo;
(iii) increased production of a neuron-associated molecule in culture or in vivo, e.g., choline acetyltransferase or acetylcholinesterase with respect to motor neurons; or
(iv) decreased symptoms of neuron dysfunction in vivo. Such effects may be measured by any method known in the art. In preferred, non-limiting embodiments, increased survival of neurons may be measured by the method set forth in Arakawa et al. (1990, J. Neurosci. 10:3507-3515); increased sprouting of neurons may be detected by methods set forth in Pestronk et al. (1980, Exp. Neurol. 70:65-82) or Brown et al. (1981, Ann. Rev. Neurosci. 4:17-42); increased production of neuron-associated molecules may be measured by bioassay, enzymatic assay, antibody binding, Northern blot assay, etc., depending on the molecule to be measured; and motor neuron dysfunction may be measured by assessing the physical manifestation of motor neuron disorder, e.g., weakness, motor neuron conduction velocity, or functional disability.

In a specific embodiments, motor neuron disorders that may be treated according to the invention include but are not limited to disorders such as infarction, infection, exposure to toxin, trauma, surgical damage, degenerative disease or malignancy that may affect motor neurons as well as other components of the nervous system, as well as disorders that selectively affect neurons such as amyotrophic lateral sclerosis, and including but not limited to progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

### 5.1.3. Tissue Repair and Regeneration

In another embodiment of the invention, a Therapeutic of the invention is used for promotion of tissue regeneration and repair, including but not limited to treatment of benign dysproliferative disorders. Specific embodiments are directed to treatment of cirrhosis of the liver (a condition in which scarring has overtaken normal liver regeneration processes), treatment of keloid (hypertrophic scat) formation (disfiguring of the skin in which the scarring process interferes with normal renewal), psoriasis (a common skin condition characterized by excessive proliferation of the skin and delay in proper cell fate determination), and baldness (a condition in which terminally differentiated hair follicles (a tissue rich in Notch) fail to function properly).

### 5.2. Prophylactic Uses

### 5.2.1. Malignancies

The Therapeutics of the invention can be administered to prevent progression to a neoplastic or malignant state, including but not limited to those disorders listed in Table 1. Such administration is indicated where the Therapeutic is shown in assays, as described supra, to have utility for treatment or prevention of such disorder. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.) Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a Therapeutic of the invention. As mentioned supra, such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc.: (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

In other embodiments, a patient which exhibits one or more of the following predisposing factors for malignancy is treated by administration of an effective amount of a Therapeutic: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W. B. Saunders Co., Philadelphia, pp. 112-113) etc.)

In another specific embodiment, an Antagonist Therapeutic of the invention is administered to a human patient to prevent progression to breast, colon, or cervical cancer.

### 5.2.2. Other Disorders

In other embodiments, a Therapeutic of the invention can be administered to prevent a nervous system disorder described in Section 5.1.2, or other disorder (e.g., liver cirrhosis, psoriasis, keloids, baldness) described in Section 5.1.3.

### 5.3. Demonstration of Therapeutic or Prophylactic Utility

The Therapeutics of the invention can be tested in vivo for the desired therapeutic or prophylactic activity. For example, such compounds can be tested in suitable animal model systems prior to testing in humans, including but not limited to rats, mice, chicken, cows, monkeys, rabbits, etc. For in vivo testing, prior to administration to humans, any animal model system known in the art may be used.

### 5.4. Therapeutic/Prophylactic Administration and Compositions

The invention provides methods of treatment (and prophylaxis) by administration to a subject of an effective amount of a Therapeutic of the invention. In a preferred aspect, the Therapeutic is substantially purified. The subject is preferably an animal, including but not limited to animals such as cows, pigs, chickens, etc., and is preferably a mammal, and most preferably human.

Various delivery systems are known and can be used to administer a Therapeutic of the invention, e.g., encapsulation in liposomes, microparticles, microcapsules, expression by recombinant cells, receptor-mediated endocytosis (see, e.g., Wu and Wu, 1987, J. Biol. Chem. 262:4429-4432), construction of a Therapeutic nucleic acid as part of a retroviral or other vector, etc. Methods of introduction include but are not limited to intradermal, intramuscular, intraperitoneal, intravenous, subcutaneous, intranasal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In a specific embodiment, administration of a Therapeutic into a Notch-expressing cell is accomplished by linkage of the Therapeutic to a Delta (or other toporythmic) protein or portion thereof capable of mediating binding to Notch. Contact of a Notch-expressing cell with the linked Therapeutic results in binding of the linked Therapeutic via its Delta portion to Notch on the surface of the cell, followed by uptake of the linked Therapeutic into the Notch-expressing cell.

In a specific embodiment wherein an analog of a Notch intracellular signal-transducing domain is employed as a Therapeutic, such that it can inhibit Notch signal transduction, the analog is preferably delivered intracellularly (e.g., by expression from a nucleic acid vector, or by linkage to a Delta protein capable of binding to Notch followed by binding and internalization, or by receptor-mediated mechanisms).

In a specific embodiment where the Therapeutic is a nucleic acid encoding a protein Therapeutic, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid Therapeutic can be introduced intracellularly and incorporated within host cell DNA for expression, by homologous recombination.

In specific embodiments directed to treatment or prevention of particular disorders, preferably the following forms of administration are used:

| Disorder | Preferred Forms of Administration |
|---|---|
| Cervical cancer | Topical |
| Gastrointestinal cancer | Oral; intravenous |
| Lung cancer | Inhaled; intravenous |
| Leukemia | Intravenous; extracorporeal |
| Metastatic carcinomas | Intravenous; oral |
| Brain cancer | Targeted; intravenous; intrathecal |
| Liver cirrhosis | Oral; intravenous |
| Psoriasis | Topical |
| Keloids | Topical |
| Baldness | Topical |
| Spinal cord injury | Targeted; intravenous; intrathecal |
| Parkinson's disease | Targeted; intravenous; intrathecal |
| Motor neuron disease | Targeted; intravenous; intrathecal |
| Alzheimer's disease | Targeted; intravenous; intrathecal |

The present invention also provides pharmaceutical compositions. Such compositions comprise a therapeutically effective amount of a Therapeutic, and a pharmaceutically acceptable carrier or excipient. Such a carrier includes but is not limited to saline, buffered saline, dextrose, water, glycerol, ethanol, and combinations thereof. The carrier and composition can be sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.5. Antisense Regulation of Notch Expression

The present invention provides the therapeutic or prophylactic use of nucleic acids of at least six nucleotides that are antisense to a gene or cDNA encoding Notch or a portion thereof. “Antisense” as used herein refers to a nucleic acid capable of hybridizing to a portion of a Notch RNA (preferably mRNA) by virtue of some sequence complementarity. Such antisense nucleic acids have utility as Antagonist Therapeutics of the invention, and can be used in the treatment or prevention of disorders as described supra in Section 5.1 and its subsections.

The antisense nucleic acids of the invention can be oligonucleotides that are double-stranded or single-stranded, RNA or DNA or a modification or derivative thereof, which can be directly administered to a cell, or which can be produced intracellularly by transcription of exogenous, introduced sequences.

In a specific embodiment, the Notch antisense nucleic acids provided by the instant invention can be used for the treatment of tumors or other disorders, the cells of which tumor type or disorder can be demonstrated (in vitro or in vivo) to express the Notch gene. Such demonstration can be by detection of Notch RNA or of Notch protein.

The invention further provides pharmaceutical compositions comprising an effective amount of the Notch antisense nucleic acids of the invention in a pharmaceutically acceptable carrier, as described supra in Section 5.4. Methods for treatment and prevention of disorders (such as those described in Sections 5.1 and 5.2) comprising administering the pharmaceutical compositions of the invention are also provided.

In another embodiment, the invention is directed to methods for inhibiting the expression of a Notch nucleic acid sequence in a prokaryotic or eukaryotic cell comprising providing the cell with an effective amount of a composition comprising an antisense Notch nucleic acid of the invention.

In another embodiment, the identification of cells expressing functional Notch receptors can be carried out by observing the ability of Notch to “rescue” such cells from the cytotoxic effects of a Notch antisense nucleic acid.

In an alternative embodiment of the invention, nucleic acids antisense to a nucleic acid encoding a (“adhesive”) toporythmic protein or fragment that binds to Notch, are envisioned as Therapeutics.

Notch antisense nucleic acids and their uses are described in detail below.

### 5.5.1. Notch Antisense Nucleic Acids

The Notch antisense nucleic acids are of at least six nucleotides and are preferably oligonucleotides (ranging from 6 to about 50 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549).

In a preferred aspect of the invention, a Notch antisense oligonucleotide is provided, preferably of single-stranded DNA. In a most preferred aspect, such an oligonucleotide comprises a sequence antisense to the sequence encoding ELR 11 and ELR 12 of Notch, most preferably, of human Notch. The oligonucleotide may be modified at any position on its structure with substituents generally known in the art.

The Notch antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units; the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g. by use of an automated DNA synthesizer (such as are commercially available from Biosearch, Applied Biosystems, etc.). As examples, phosphorothioate oligos may be synthesized by the method of Stein et al. (1988, Nucl. Acids Res. 16:3209), methylphosphonate oligos can be prepared by use of controlled pore glass polymer supports (Sarin et al., 1988, Proc. Natl. Acad. Sci. U.S.A. 85:7448-7451), etc.

In a specific embodiment, the Notch antisense oligonucleotide comprises catalytic RNA, or a ribozyme (see, e.g., PCT International Publication WO 90/11364, published Oct. 4, 1990; Sarver et al., 1990, Science 247:1222-1225). In another embodiment, the oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

In an alternative embodiment, the Notch antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the Notch antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the Notch antisense RNA can be by any promoter known in the art to act in mammalian, preferably human, cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), etc.

The antisense nucleic acids of the invention comprise a sequence complementary to at least a portion of an RNA transcript of a Notch gene, preferably a human Notch gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded Notch antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a Notch RNA it may contain and still form a stable duplex (or triplex, as the case may be). One skilled in the art can ascertain a tolerable degree of mismatch by use of standard procedures to determine the melting point of the hybridized complex.

### 5.5.2. Therapeutic Utility of Notch Antisense Nucleic Acids

The Notch antisense nucleic acids can be used to treat (or prevent) malignancies, of a cell type which has been shown to express Notch RNA. Malignant, neoplastic, and pre-neoplastic cells which can be tested for such expression include but are not limited to those described supra in Sections 5.1.1 and 5.2.1. In a preferred embodiment, a single-stranded DNA antisense Notch oligonucleotide is used. Malignant (particularly, tumor) cell types which express Notch RNA can be identified by various methods known in the art. Such methods include but are not limited to hybridization with a Notch-specific nucleic acid (e.g. by Northern hybridization, dot blot hybridization, in situ hybridization), observing the ability of RNA from the cell type to be translated in vitro into Notch, etc. In a preferred aspect, primary tumor tissue from a patient can be assayed for Notch expression prior to treatment.

Pharmaceutical compositions of the invention (see Section 5.1.4), comprising an effective amount of a Notch antisense nucleic acid in a pharmaceutically acceptable carrier, can be administered to a patient having a malignancy which is of a type that expresses Notch RNA.

The amount of Notch antisense nucleic acid which will be effective in the treatment of a particular disorder or condition will depend on the nature of the disorder or condition, and can be determined by standard clinical techniques. Where possible, it is desirable to determine the antisense cytotoxicity of the tumor type to be treated in vitro, and then in useful animal model systems prior to testing and use in humans.

In a specific embodiment, pharmaceutical compositions comprising Notch antisense nucleic acids are administered via liposomes, microparticles, or microcapsules. In various embodiments of the invention, it may be useful to use such compositions to achieve sustained release of the Notch antisense nucleic acids. In a specific embodiment, it may be desirable to utilize liposomes targeted via antibodies to specific identifiable tumor antigens (Leonetti et al., 1990, Proc. Natl. Acad. Sci. U.S.A. 87:2448-2451; Renneisen et al., 1990, J. Biol. Chem. 265:16337-16342).

### 5.6. Diagnostic Utility

Notch proteins, analogues, derivatives, and subsequences thereof, Notch nucleic acids (and sequences complementary thereto), anti-Notch antibodies, and other toporythmic proteins and derivatives and analogs thereof which interact with Notch proteins, and inhibitors of North-toporythmic protein interactions, have uses in diagnostics. Such molecules can be used in assays, such as immunoassays, to detect, prognose, diagnose, or monitor various conditions, diseases, and disorders affecting Notch expression, or monitor the treatment thereof. In particular, such an immunoassay is carried out by a method comprising contacting a sample derived from a patient with an anti-Notch antibody under conditions such that immunospecific binding can occur, and detecting or measuring the amount of any immunospecific binding by the antibody. In a specific embodiment, antibody to Notch can be used to assay in a patient tissue or serum sample for the presence of Notch where an aberrant level of Notch is an indication of a diseased condition.

The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

Notch genes and related nucleic acid sequences and subsequences, including complementary sequences, and other toporythmic gene sequences, can also be used in hybridization assays. Notch nucleic acid sequences, or subsequences thereof comprising about at least 8 nucleotides, can be used as hybridization probes. Hybridization assays can be used to detect, prognose, diagnose, or monitor conditions, disorders, or disease states associated with aberrant changes in Notch expression and/or activity as described supra. In particular, such a hybridization assay is carried out by a method comprising contacting a sample containing nucleic acid with a nucleic acid probe capable of hybridizing to Notch DNA or RNA, under conditions such that hybridization can occur, and detecting or measuring any resulting hybridization.

As detailed in examples section 10.1 infra, increased Notch expression occurs in human breast, colon, and cervical cancer. Accordingly, in specific embodiments, human breast, colon, or cervical cancer or premalignant changes in such tissues is diagnosed by detecting increased Notch expression in patient samples relative to the level of Notch expression in an analogous non-malignant sample (from the patient or another person, as determined experimentally or as is known as a standard level in such samples).

In one embodiment, the Notch protein (or derivative having Notch antigenicity) that is detected or measured is on the cell surface. In another embodiment, the Notch protein (or derivative) is a cell free soluble molecule (e.g., as measured in a blood or serum sample) or is intracellular. Without intending to be bound mechanistically, Applicants believe that cell free Notch may result from secretion or shedding from the cell surface. In yet another embodiment, soluble, cell-surface, and intracellular amounts of Notch protein or derivative are detected or measured.

### 5.7. Notch Nucleic Acids

Therapeutics of the invention which are Notch nucleic acids or Notch antisense nucleic acids, as well as nucleic acids encoding protein Therapeutics, include those described below, which can be obtained by methods known in the art, and in particular, as described below.

In particular aspects, the invention provides amino acid sequences of Notch, preferably human Notch, and fragments and derivatives thereof which comprise an antigenic determinant (i.e., can be recognized by an antibody) or which are functionally active, as well as nucleic acid sequences encoding the foregoing. “Functionally active” material as used herein refers to that material displaying one or more known functional activities associated with the full-length (wild-type) Notch protein product, e.g., binding to Delta, binding to Serrate, binding to any other Notch ligand, antigenicity (binding to an anti-Notch antibody), etc.

In specific embodiments, the invention provides fragments of a Notch protein consisting of at least 40 amino acids, or of at least 75 amino acids. In other embodiments, the proteins comprise or consist essentially of the intracellular domain, transmembrane region, extracellular domain, cdc10 region, Notch/lin-12 repeats, or the EGF-homologous repeats, or any combination of the foregoing, of a Notch protein. Fragments, or proteins comprising fragments, lacking some or all of the EGF-homologous repeats of Notch are also provided. Nucleic acids encoding the foregoing are provided.

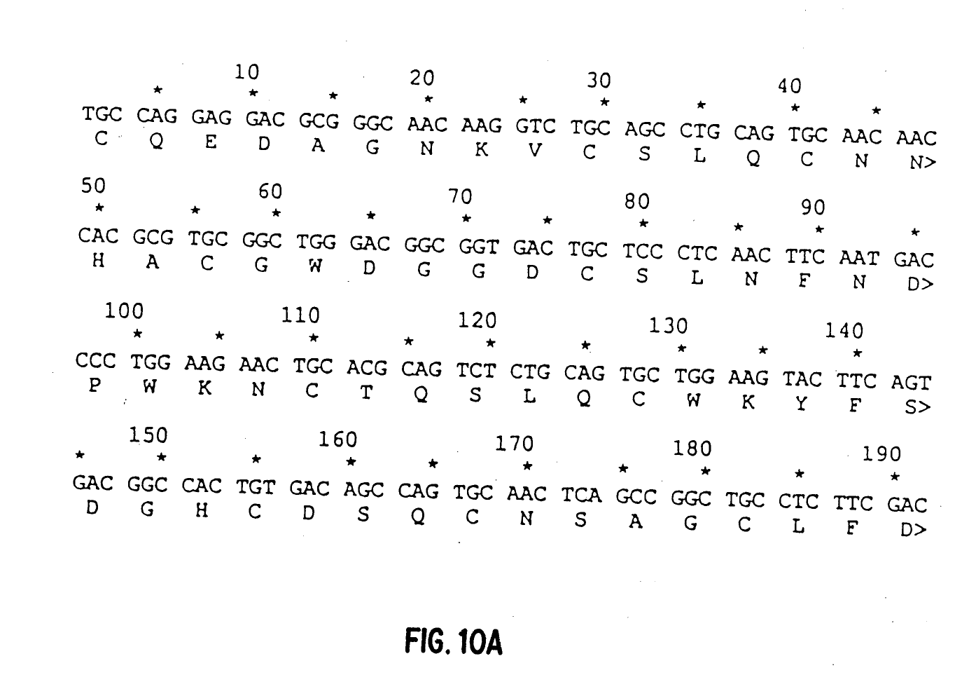

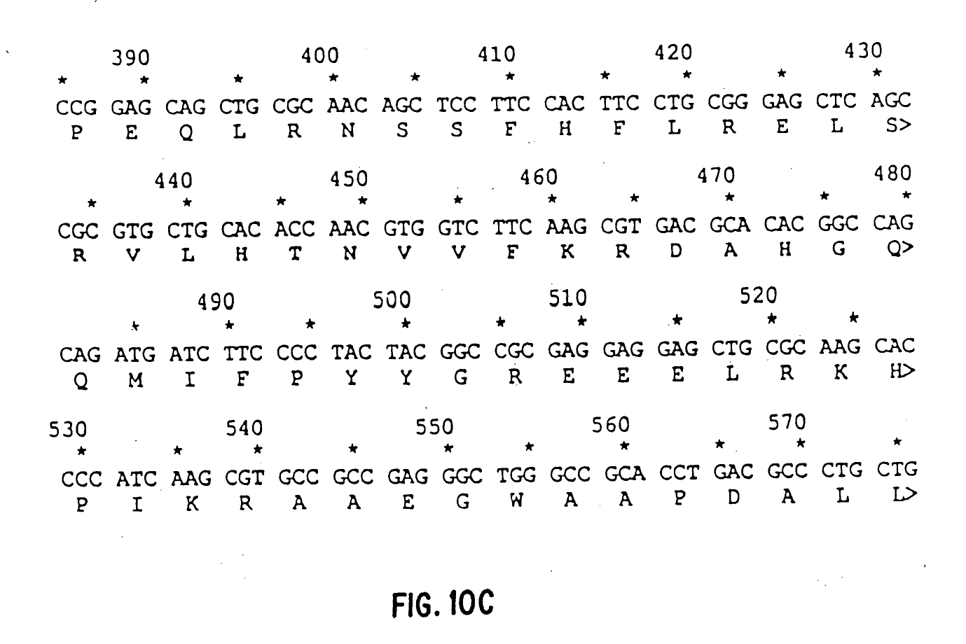

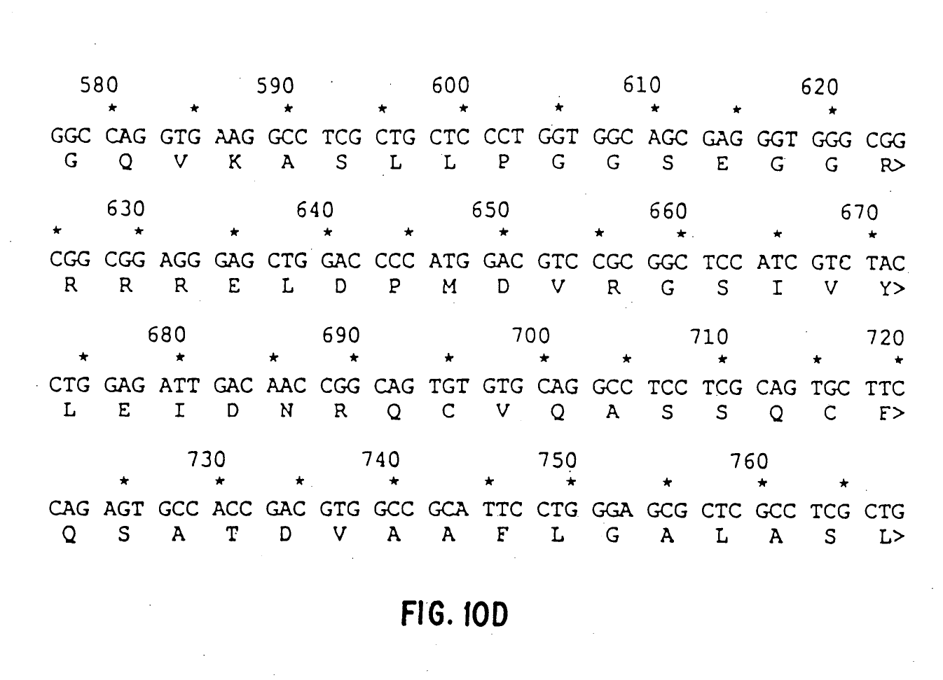

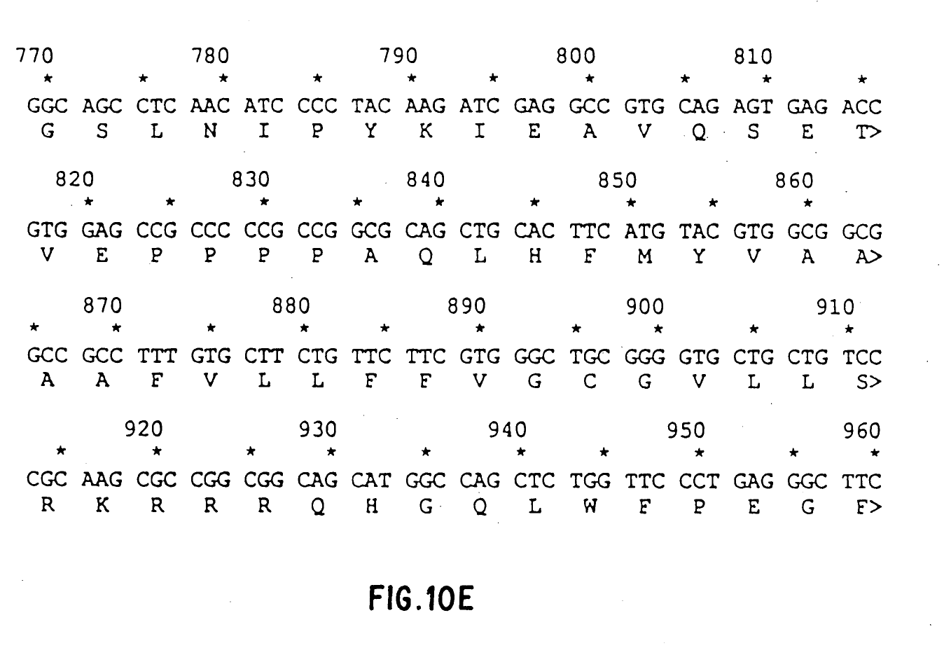

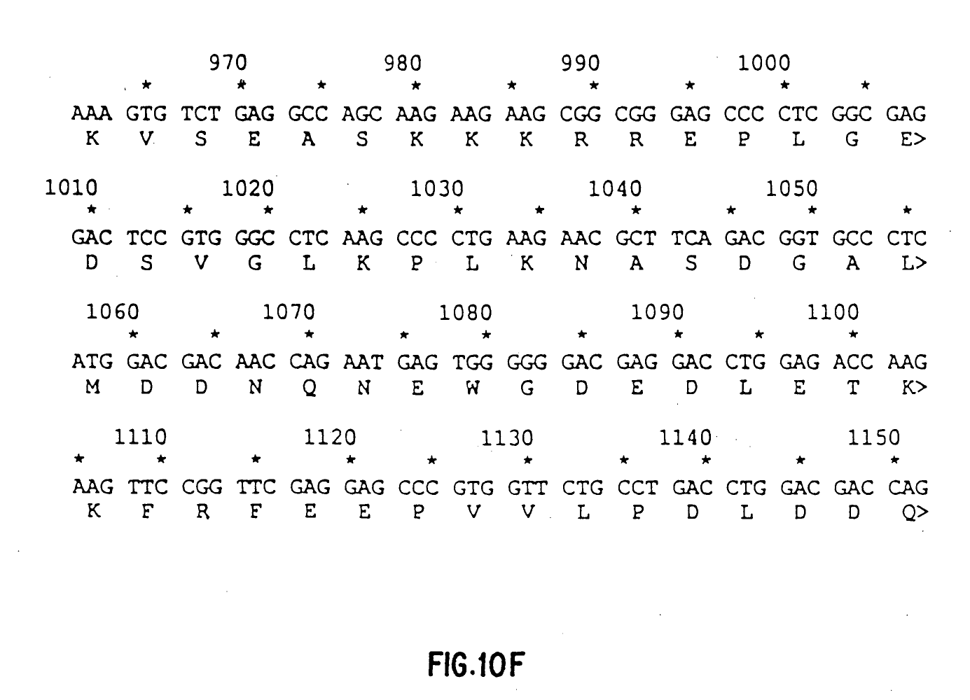

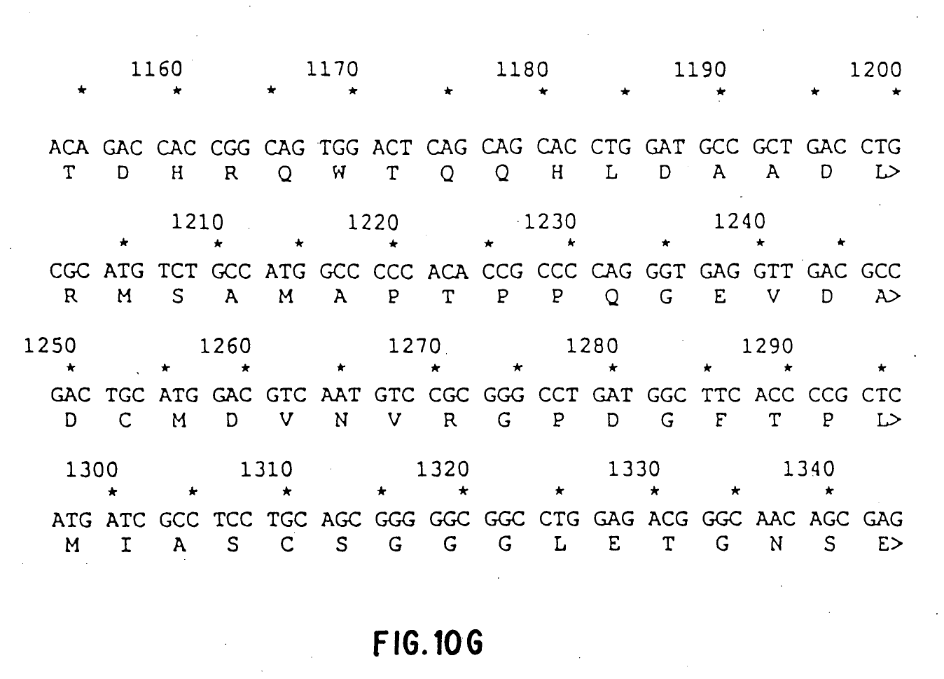

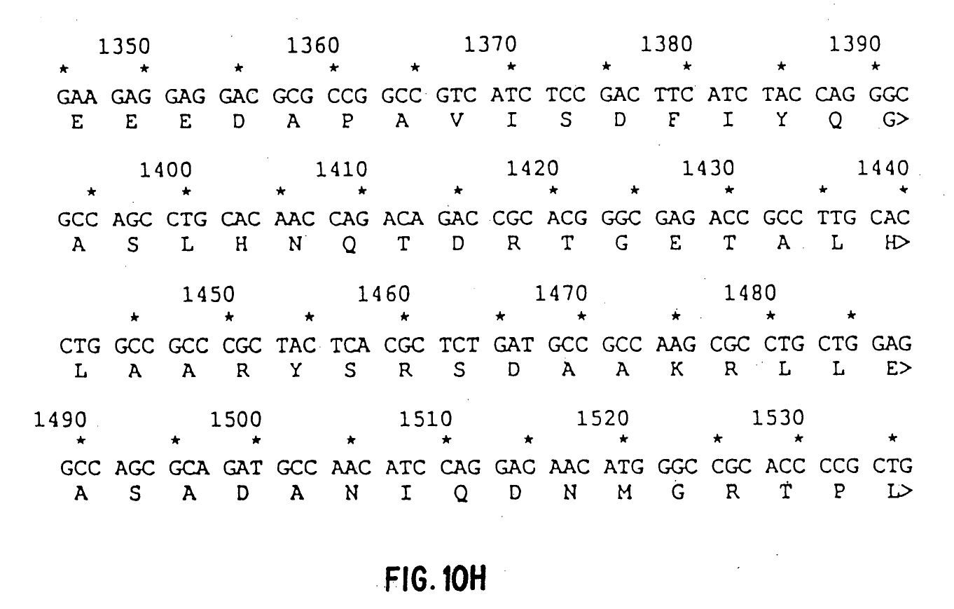

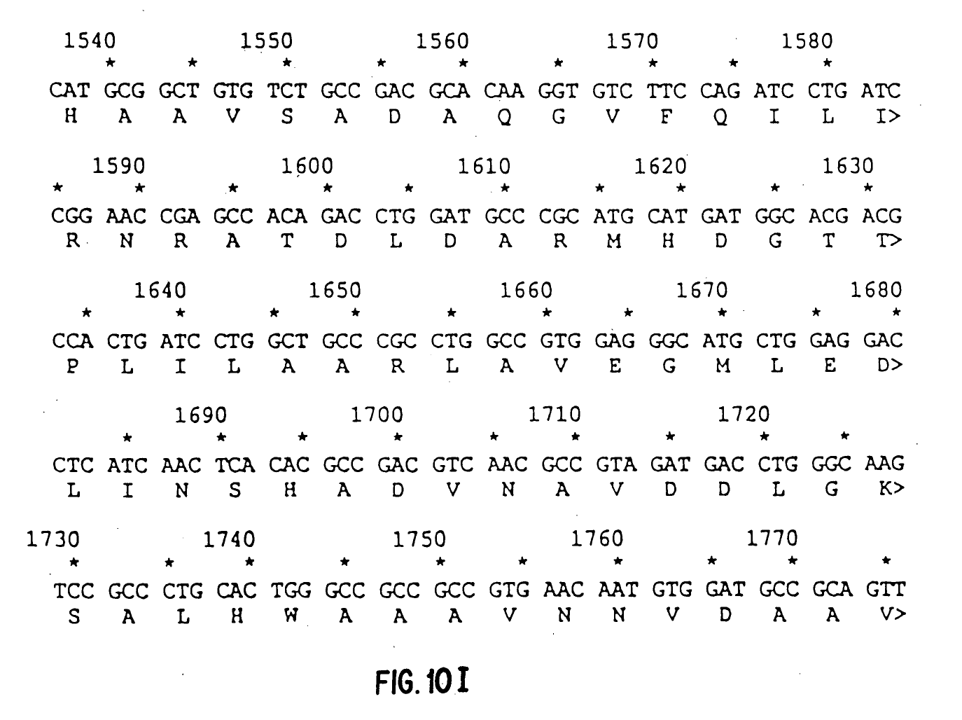

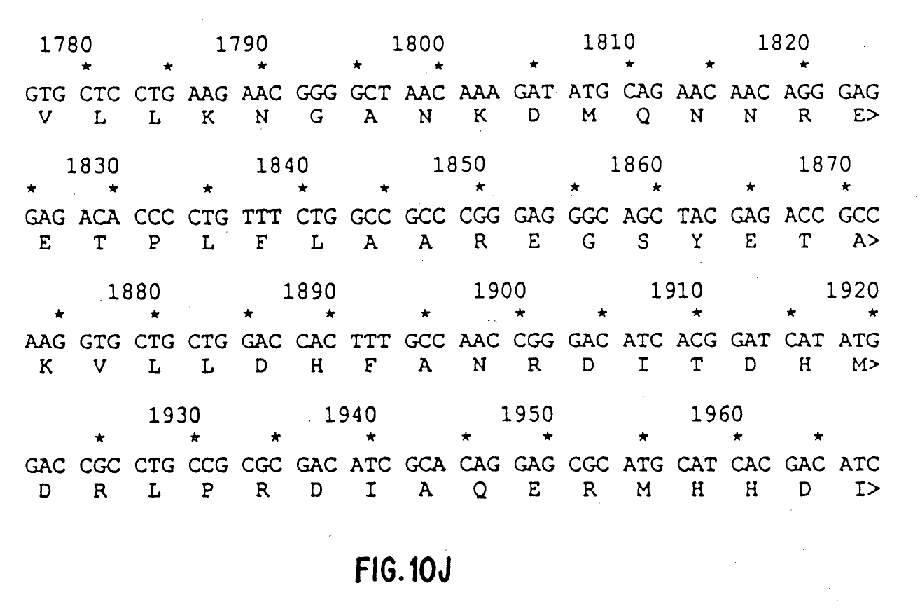

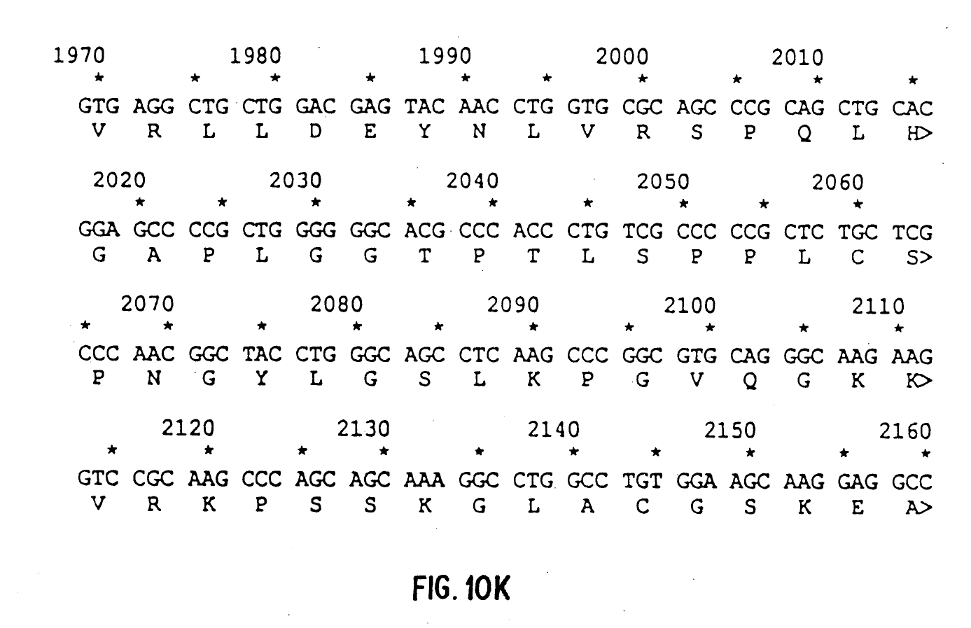

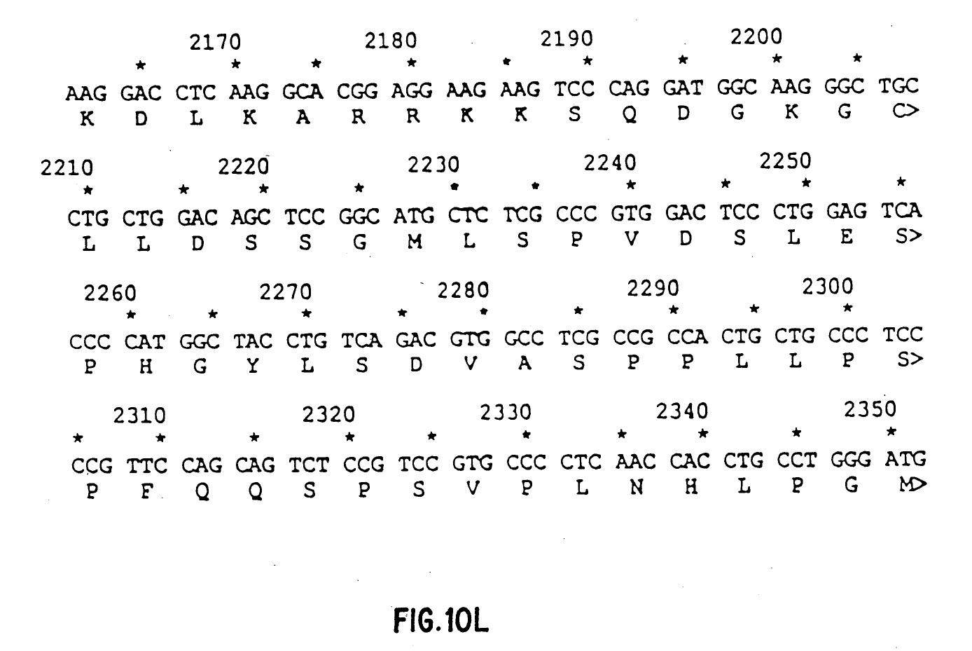

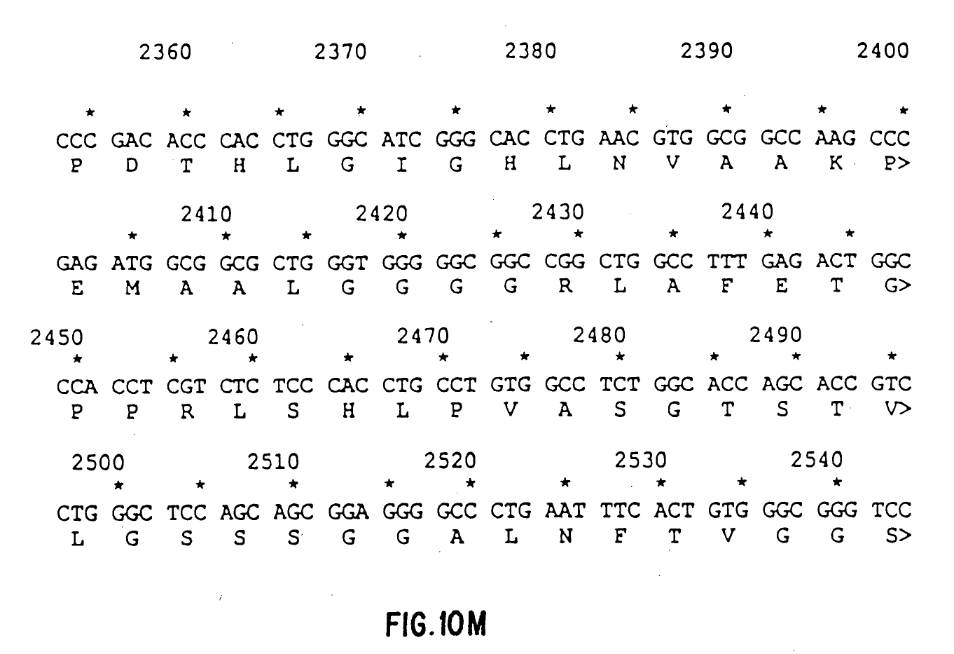

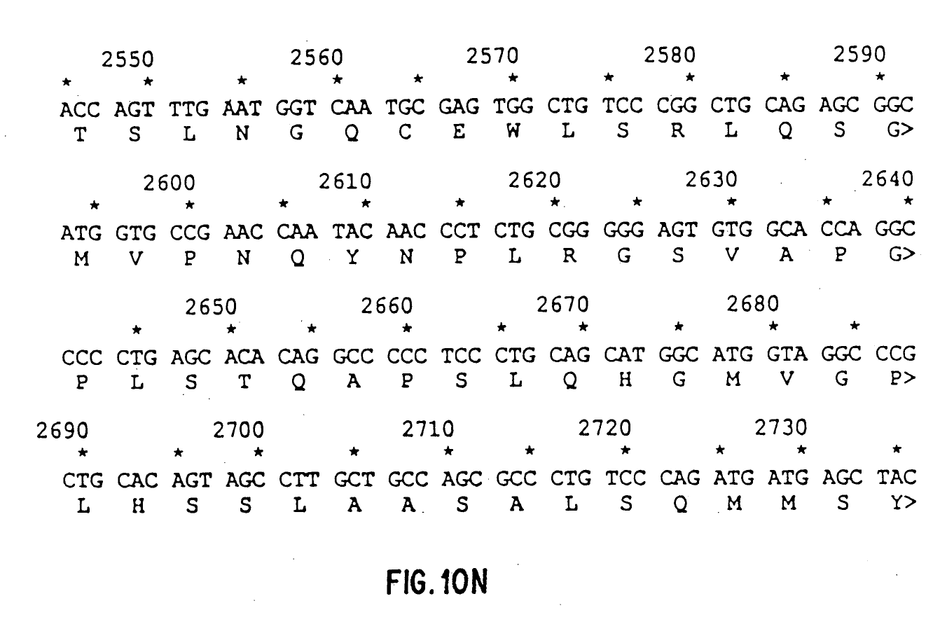

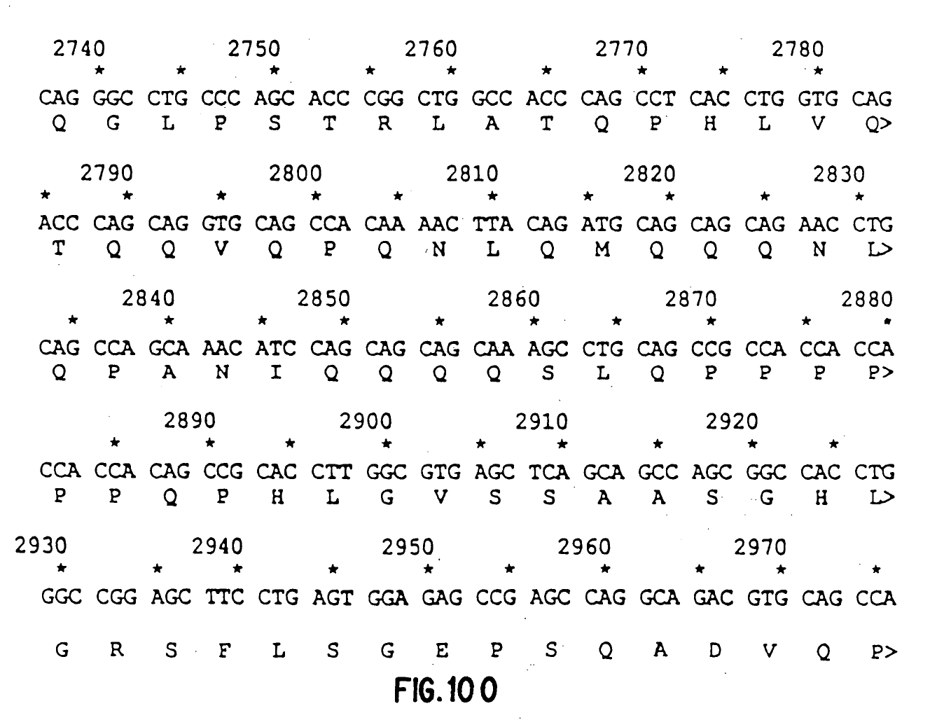

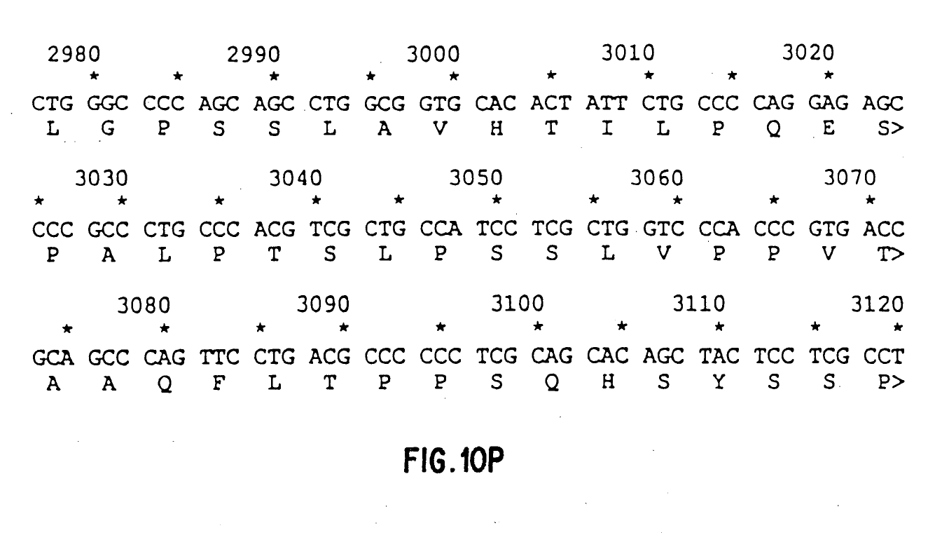

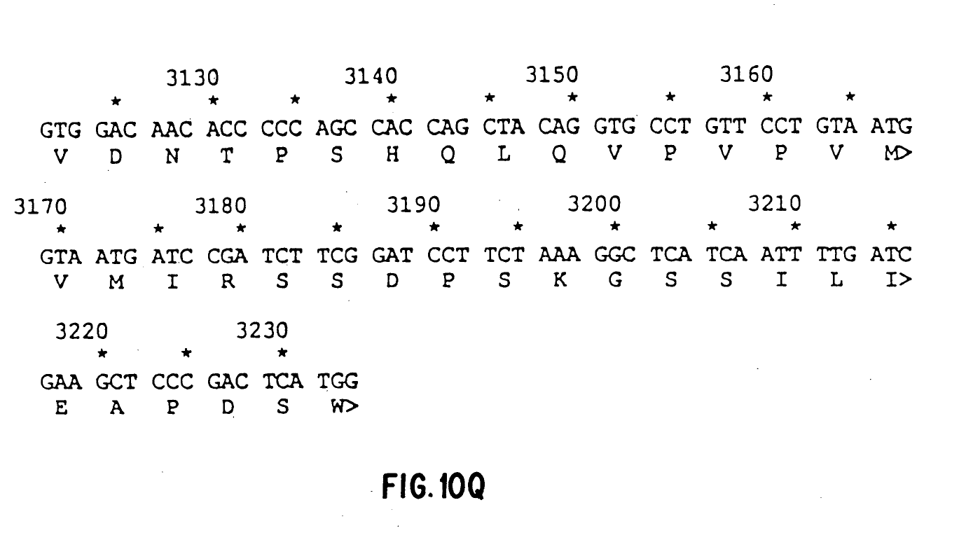

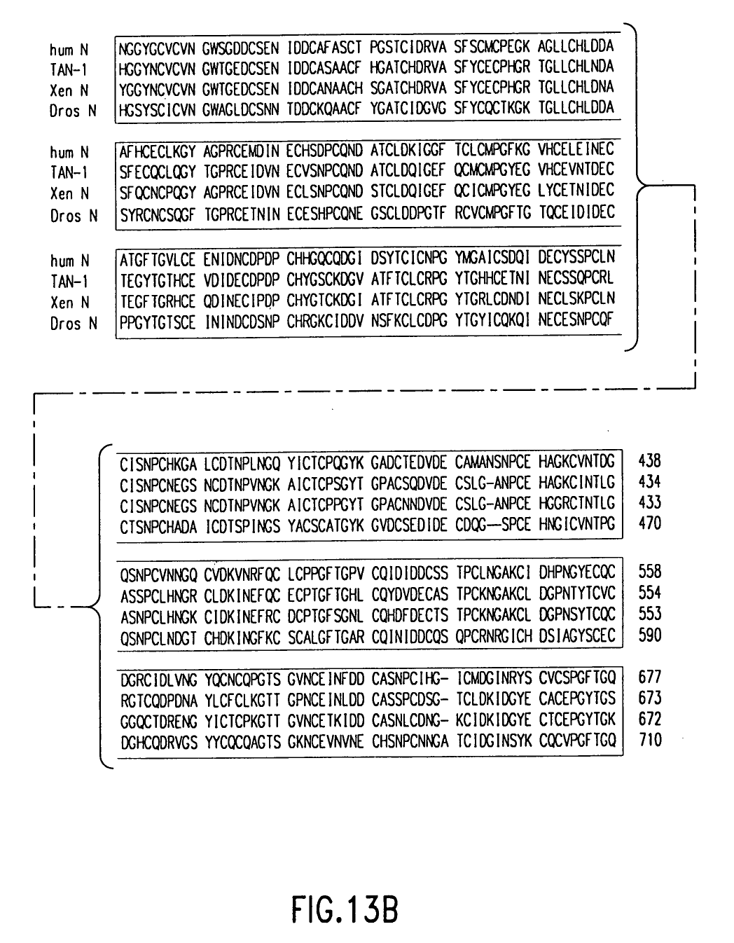

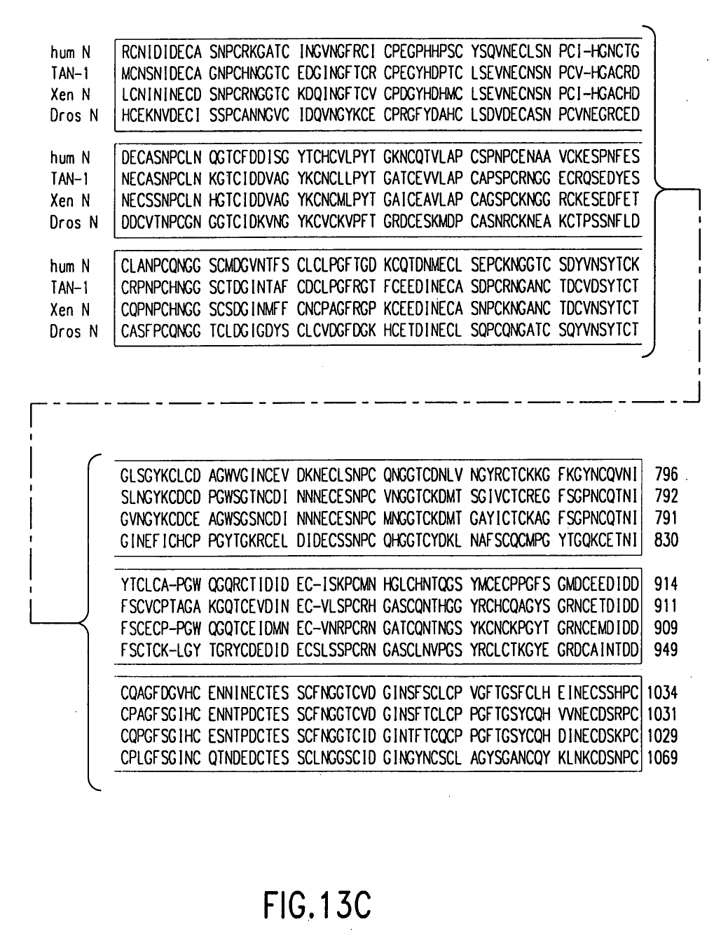

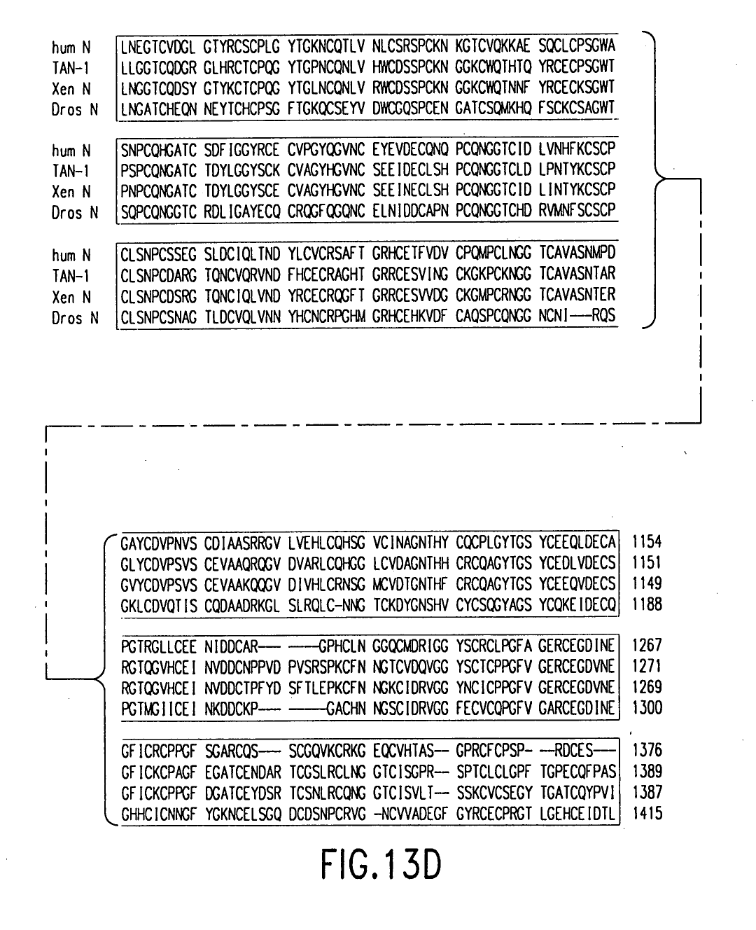

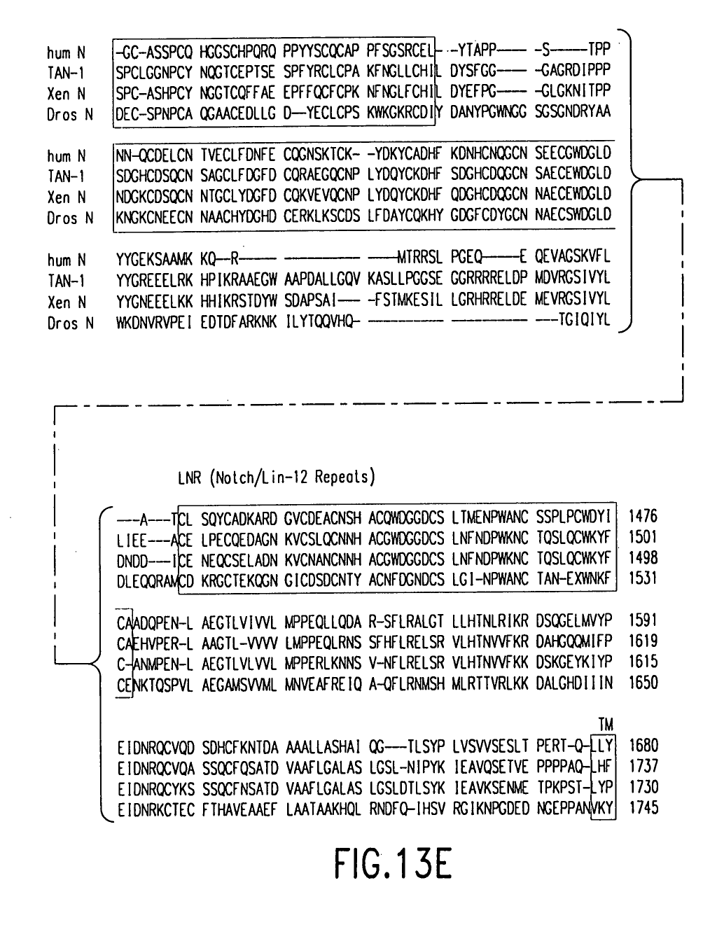

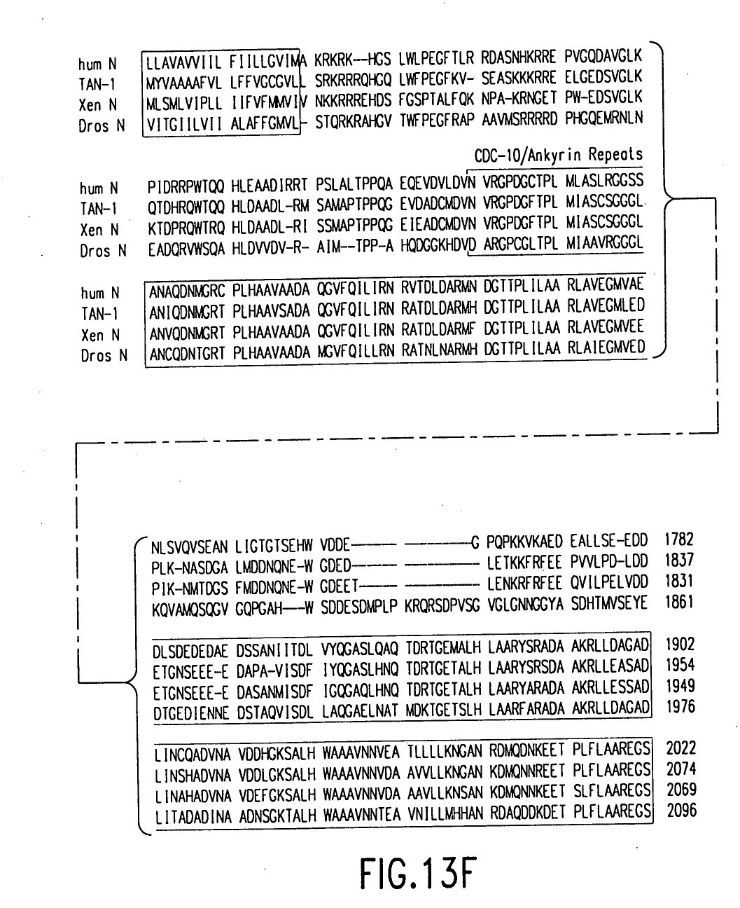

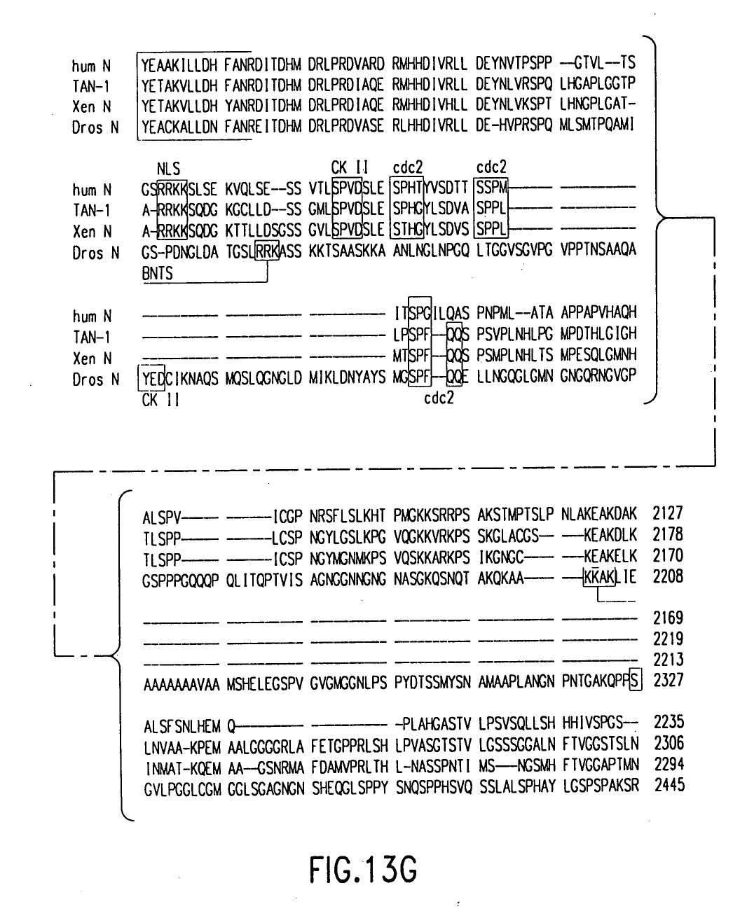

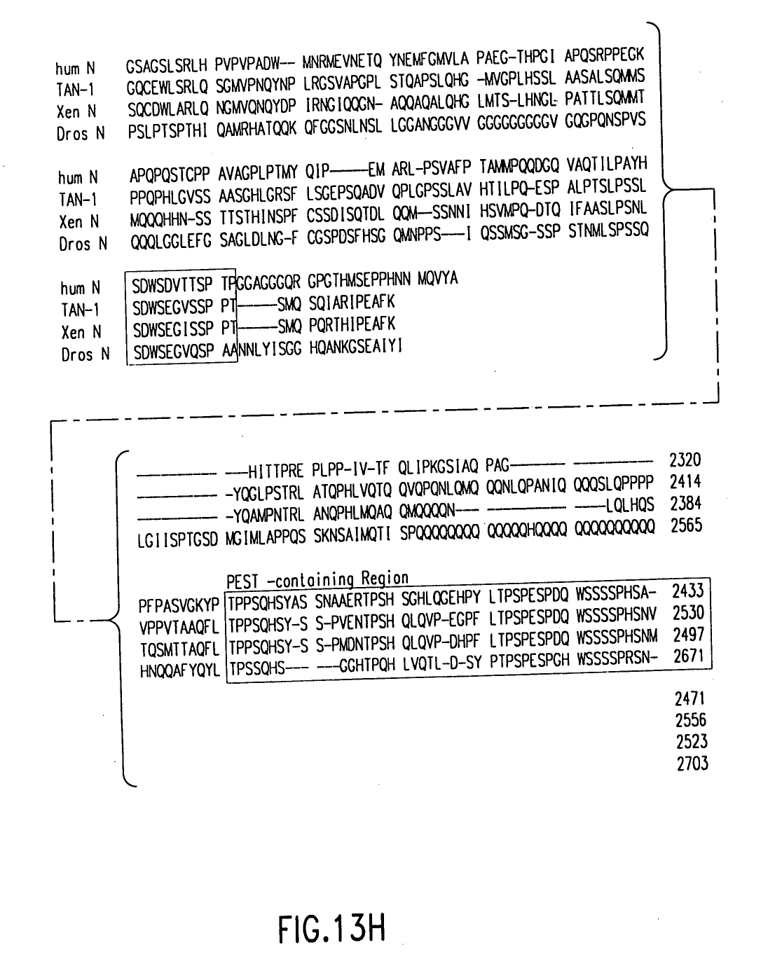

In other specific embodiments, the invention provides nucleotide sequences and subsequences of Notch, preferably human Notch, consisting of at least 25 nucleotides, at least 50 nucleotides, or at least 150 nucleotides. Nucleic acids encoding the proteins and protein fragments described above are provided, as well as nucleic acids complementary to and capable of hybridizing to such nucleic acids. In one embodiment, such a complementary sequence may be complementary to a Notch cDNA sequence of at least 25 nucleotides, or of at least 100 nucleotides. In a preferred aspect, the invention utilizes cDNA sequences encoding human Notch or a portion thereof. In a specific embodiment, such sequences of the human Notch gene or cDNA are as contained in plasmids hN3k, hN4k, or hN5k (see Section 9, infra) or in the gene corresponding thereto; such a human Notch protein sequence can be as shown in FIG. 10 (SEQ ID NO:11) or 11 (SEQ ID NO:13). In other embodiments, the Notch nucleic acid and/or its encoded protein has at least a portion of the sequence shown in one of the following publications: Wharton et al., 1985, Cell 43:567-581 (Drosophila Notch); Kidd et al., 1986, Mol. Cell. Biol. 6:3094-3108 (Drosophila Notch); Coffman et al., 1990, Science 249:1438-1441 (Xenopus Notch); Ellisen et al., 1991, Cell 66:649-661 (a human Notch). In another aspect, the sequences of human Notch are those encoding the human Notch amino acid sequences or a portion thereof as shown in FIG. 13. In a particular aspect, the human Notch sequences are those of the hN homolog (represented in part by plasmid hN5k) or the TAN-1 homolog.

In one embodiment of the invention, the invention is directed to the full-length human Notch protein encoded by the hN homolog as depicted in FIG. 13, both containing the signal sequence (ie., the precursor protein; amino acids 1-2169) and lacking the signal sequence (i.e., the mature protein; amino acids ˜26-2169), as well as portions of the foregoing (e.g., the extracellular domain, EGF homologous repeat region, EGF-like repeats 11 and 12, cdc-10/ankyrin repeats, etc.) and proteins comprising the foregoing, as well as nucleic acids encoding the foregoing.

As is readily apparent, as used herein, a “nucleic acid encoding a fragment or portion of a Notch protein” shall be construed as referring to a nucleic acid encoding only the recited fragment or portion of the Notch protein and not other portions of the Notch protein.

In a preferred, but not limiting, aspect of the invention, a human Notch DNA sequence can be cloned and sequenced by the method described in Section 9, infra.

In another preferred aspect, PCR is used to amplify the desired sequence in the library, prior to selection. For example, oligonucleotide primers representing part of the adhesive domains encoded by a homologue of the desired gene can be used as primers in PCR.

The above-methods are not meant to limit the following general description of methods by which clones of Notch may be obtained.

Any eukaryotic cell can potentially serve as the nucleic acid source for the molecular cloning of the Notch gene. The DNA may be obtained by standard procedures known in the art from cloned DNA (e.g., a DNA “library”), by chemical synthesis, by cDNA cloning, or by the cloning of genomic DNA, or fragments thereof, purified from the desired human cell (see, for example Sambrook et al., 1989, Molecular Cloning, A Laboratory Manual, Cold Spring Harbor Laboratory, 2d. Ed., Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, if an amount of a portion of a Notch (of any species) gene or its specific RNA, or a fragment thereof e.g., the adhesive domain, is available and can be purified and labeled, the generated DNA fragments may be screened by nucleic acid hybridization to the labeled probe (Benton, W. and Davis, R., 1977, Science 196, 180; Grunstein, M. And Hogness, D., 1975, Proc. Natl. Acad. Sci. U.S.A. 72, 3961). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene. Alternatively, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isolectric focusing behavior, proteolytic digestion maps, in vitro aggregation activity (“adhesiveness”) or antigenic properties as known for Notch. If an antibody to Notch is available, the Notch protein may be identified by binding of labeled antibody to the putatively Notch synthesizing clones, in an ELISA (enzyme-linked immunosorbent assay)-type procedure.

The Notch gene can also be identified by mRNA selection by nucleic acid hybridization followed by in vitro translation. In this procedure, fragments are used to isolate complementary mRNAs by hybridization. Such DNA fragments may represent available, purified Notch DNA of another species (e.g., Drosophila). Immunoprecipitation analysis or functional assays (e.g., aggregation ability in vitro; see examples infra) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against Notch or Delta protein. A radiolabelled Notch cDNA can be synthesized using the selected mRNA (from the adsorbed polysomes) as a template. The radiolabelled mRNA or cDNA may then be used as a probe to identify the Notch DNA fragments from among other genomic DNA fragments.

Alternatives to isolating the Notch genomic DNA include, but are not limited to, chemically synthesizing the gene sequence itself from a known sequence or making cDNA to the mRNA which encodes the Notch gene. For example, RNA for cDNA cloning of the Notch gene can be isolated from cells which express Notch. Other methods are possible and within the scope of the invention.

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and Notch or Delta gene may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shot gun” approach. Enrichment for the desired gene, for example, by size fractionization, can be done before insertion into the cloning vector.

In specific embodiments, transformation of host cells with recombinant DNA molecules that incorporate the isolated Notch gene, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The Notch sequences provided by the instant invention include those nucleotide sequences encoding substantially the same amino acid sequences as found in native Notch protein, and those encoded amino acid sequences with functionally equivalent amino acids, all as described in Section 5.6 infra for Notch derivatives.

Similar methods to those described supra can be used to obtain a nucleic acid encoding Delta, Serrate, or adhesive portions thereof, or other toporythmic gene of interest. In a specific embodiment, the Delta nucleic acid has at least a portion of the sequence shown in FIG. 1 (SEQ ID NO:1). In another specific embodiment, the Serrate nucleic acid has at least a portion of the sequence shown in FIG. 5 (SEQ ID NO:3). The nucleic acid sequences encoding toporythmic proteins can be isolated from porcine, bovine, feline, avian, equine, or canine, as well as primate sources and any other species in which homologs of known toporythmic genes [including but not limited to the following genes (with the publication of sequences in parentheses): Delta (Vassin et al., 1987, EMBO J. 6, 3431-3440; Kopczynski et al., 1988, Genes Dev. 2, 1723-1735; note corrections to the Kopczynski et al. sequence found in FIG. 1 hereof (SEQ ID NO:1 and SEQ ID NO:2)) and Serrate (Fleming et al., 1990, Genes & Dev. 4, 2188-2201)] can be identified. Such sequences can be altered by substitutions, additions or deletions that provide for functionally equivalent molecules, as described supra.

### 5.8. Recombinant Production of Protein Therapeutics

The nucleic acid coding for a protein Therapeutic of the invention can be inserted into an appropriate expression vector, i.e., a vector which contains the necessary elements for the transcription and translation of the inserted protein-coding sequence. The necessary transcriptional and translational signals can also be supplied by the native toporythmic gene and/or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In a specific embodiment, the adhesive portion of the Notch gene, e.g., that encoding EGF-like repeats (ELR) 11 and 12, is expressed. In other specific embodiments, the human Notch gene is expressed, or a sequence encoding a functionally active portion of human Notch.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of nucleic acid sequence encoding a Notch protein or peptide fragment may be regulated by a second nucleic acid sequence so that the Notch protein or peptide is expressed in a host transformed with the recombinant DNA molecule. For example, expression of a Notch protein may be controlled by any promoter/enhancer element known in the art. Promoters which may be used to control toporythmic gene expression include, but are not limited to, the SV40 early promoter region (Bernoist and Chambon, 1981, Nature 290, 304-310), the promoter contained in the 3′ long terminal repeat of Rous sarcoma virus (Yamamoto, et al., 1980, Cell 22, 787-797), the herpes thymidine kinase promoter (Wagner et al., 1981, Proc. Natl. Acad. Sci. U.S.A. 78, 1441-1445), the regulatory sequences of the metallothionein gene (Brinster et al., 1982, Nature 296, 39-42); prokaryotic expression vectors such as the β-lactamase promoter (Villa-Kamaroff, et al., 1978, Proc. Natl. Acad. Sci. U.S.A. 75, 3727-3731), or the tac promoter (DeBoer, et al., 1983, Proc. Natl. Acad. Sci. U.S.A. 80, 21-25); see also “Useful proteins from recombinant bacteria” in Scientific American, 1980, 242, 74-94; plant expression vectors comprising the nopaline synthetase promoter region (Herrera-Estrella et al., Nature 303, 209-213) or the cauliflower mosaic virus 35S RNA promoter (Gardner, et al., 1981, Nucl. Acids Res. 9, 2871), and the promoter of the photosynthetic enzyme ribulose biphosphate carboxylase (Herrera-Estrella et al., 1984, Nature 310, 115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38, 639-646; Ornitz et al., 1986, Cold Spring Harbor Symp. Quant. Biol. 50, 399409; MacDonald, 1987, Hepatology 7, 425-515); insulin gene control region which is active in pancreatic beta cells (Hanahan, 1985, Nature 315, 115-122), immunoglobulin gene control region which is active in lymphoid cells (Grosschedl et al., 1984, Cell 38, 647-658; Adames et al., 1985, Nature 318, 533-538; Alexander et al., 1987, Mol. Cell. Biol. 7, 1436-1444), mouse mammary tumor virus control region which is active in testicular, breast, lymphoid and mast cells (Leder et al., 1986, Cell 45, 485-495), albumin gene control region which is active in liver (Pinkert et al., 1987, Genes and Devel. 1, 268-276), alpha-fetoprotein gene control region which is active in liver (Krumlauf et al., 1985, Mol. Cell. Biol. 5, 1639-1648; Hammer et al., 1987, Science 235, 53-58; alpha 1-antitrypsin gene control region which is active in the liver (Kelsey et al., 1987, Genes and Devel. 1, 161-171), beta-globin gene control region which is active in myeloid cells (Mogram et al., 1985, Nature 315, 338-340; Kollias et al., 1986, Cell 46, 89-94; myelin basic protein gene control region which is active in oligodendrocyte cells in the brain (Readhead et al., 1987, Cell 48, 703-712); myosin light chain-2 gene control region which is active in skeletal muscle (Sani, 1985, Nature 314, 283-286), and gonadotropic releasing hormone gene control region which is active in the hypothalamus (Mason et al., 1986, Science 234, 1372-1378).

Expression vectors containing Notch gene inserts can be identified by three general approaches: (a) nucleic acid hybridization, (b) presence or absence of “marker” gene functions, and (c) expression of inserted sequences. In the first approach, the presence of a foreign gene inserted in an expression vector can be detected by nucleic acid hybridization using probes comprising sequences that are homologous to an inserted toporythmic gene. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. For example, if the Notch gene is inserted within the marker gene sequence of the vector, recombinants containing the Notch insert can be identified by the absence of the marker gene function. In the third approach, recombinant expression vectors can be identified by assaying the foreign gene product expressed by the recombinant. Such assays can be based, for example, on the physical or functional properties of the Notch gene product in vitro assay systems, e.g., aggregation (adhesive) ability (see Sections 6-7, infra).

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered Notch protein may be controlled. Furthermore, different host cells have characteristic and specific mechanisms for the translational and post-translational processing and modification (e.g., glycosylation, cleavage) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous mammalian toporythmic protein. Furthermore, different vector/host expression systems may effect processing reactions such as proteolytic cleavages to different extents.

In other specific embodiments, the Notch protein, fragment, analog, or derivative may be expressed as a fusion, or chimeric protein product (comprising the protein, fragment, analog, or derivative joined via a peptide bond to a heterologous protein sequence (of a different protein)). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

Both cDNA and genomic sequences can be cloned and expressed.

In other embodiments, a Notch cDNA sequence may be chromosomally integrated and expressed. Homologous recombination procedures known in the art may be used.

### 5.8.1. Identification and Purification of the Expressed Gene Product

Once a recombinant which expresses the Notch gene sequence is identified, the gene product may be analyzed. This can be achieved by assays based on the physical or functional properties of the product, including radioactive labelling of the product followed by analysis by gel electrophoresis.

Once the Notch protein is identified, it may be isolated and purified by standard methods including chromatography (e.g., ion exchange, affinity, and sizing column chromatography), centrifugation, differential solubility, or by any other standard technique for the purification of proteins. The functional properties may be evaluated using any suitable assay, including, but not limited to, aggregation assays (see Sections 6-7).

### 5.9. Derivatives and Analogs of Notch and Other Toporythmic Proteins

The invention further provides, as Therapeutics, derivatives (including but not limited to fragments) and analogs of Notch proteins. Also provided as Therapeutics are other toporythmic proteins and derivatives and analogs thereof, or Notch ligands, in particular, which promote or, alternatively, inhibit the interactions of such other toporythmic proteins with Notch.

The production and use of derivatives and analogs related to Notch are within the scope of the present invention. In a specific embodiment, the derivative or analog is functionally active, i.e., capable of exhibiting one or more functional activities associated with a full-length, wild-type Notch protein. As one example, such derivatives or analogs which have the desired antigenicity can be used, for example, in diagnostic immunoassays as described in Section 5.3. Molecules which retain, or alternatively inhibit, a desired Notch property, e.g., binding to Delta or other toporythmic proteins, binding to a intracellular ligand, can be used therapeutically as inducers, or inhibitors, respectively, of such property and its physiological correlates. Derivatives or analogs of Notch can be tested for the desired activity by procedures known in the art, including but not limited to the assays described infra. In one specific embodiment, peptide libraries can be screened to select a peptide with the desired activity; such screening can be carried out by assaying, e.g., for binding to Notch or a Notch binding partner such as Delta.

In particular, Notch derivatives can be made by altering Notch sequences by substitutions, additions or deletions that provide for functionally equivalent molecules. Due to the degeneracy of nucleotide coding sequences, other DNA sequences which encode substantially the same amino acid sequence as a Notch gene may be used in the practice of the present invention. These include but are not limited to nucleotide sequences comprising all or portions of Notch genes which are altered by the substitution of different codons that encode a functionally equivalent amino acid residue within the sequence, thus producing a silent change. Likewise, the Notch derivatives of the invention include, but are not limited to, those containing, as a primary amino acid sequence, all or part of the amino acid sequence of a Notch protein including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Derivatives or analogs of Notch include but are not limited to those peptides which are substantially homologous to Notch or fragments thereof, or whose encoding nucleic acid is capable of hybridizing to a Notch nucleic acid sequence.

The Notch derivatives and analogs of the invention can be produced by various methods known in the art. The manipulations which result in their production can occur at the gene or protein level. For example, the cloned Notch gene sequence can be modified by any of numerous strategies known in the art (Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of Notch, care should be taken to ensure that the modified gene remains within the same translational reading frame as Notch, uninterrupted by translational stop signals, in the gene region where the desired Notch activity is encoded.

Additionally, the Notch-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C., et al., 1978, J. Biol. Chem 253:6551), use of TAB® linkers (Pharmacia), etc.

Manipulations of the Notch sequence may also be made at the protein level. Included within the scope of the invention are Notch protein fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

In addition, analogs and derivatives of Notch can be chemically synthesized. For example, a peptide corresponding to a portion of a Notch protein which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the Notch sequence. Non-classical amino acids include but are not limited to the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the Notch derivative is a chimeric, or fusion, protein comprising a Notch protein or fragment thereof fused via a peptide bond at its amino- and/or carboxy-terminus to a non-Notch amino acid sequence. In one embodiment, such a chimeric protein is produced by recombinant expression of a nucleic acid encoding the protein (comprising a Notch-coding sequence joined in-frame to a non-Notch coding sequence). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding a mature Notch protein with a heterologous signal sequence is expressed such that the chimeric protein is expressed and processed by the cell to the mature Notch protein. As another example, and not by way of limitation, a recombinant molecule can be constructed according to the invention, comprising coding portions of both Notch and another toporythmic gene, e.g., Delta. The encoded protein of such a recombinant molecule could exhibit properties associated with both Notch and Delta and portray a novel profile of biological activities, including agonists as well as antagonists. The primary sequence of Notch and Delta may also be used to predict tertiary structure of the molecules using computer simulation (Hopp and Woods, 1981, Proc. Natl. Acad. Sci. U.S.A. 78:3824-3828); Notch/Delta chimeric recombinant genes could be designed in light of correlations between tertiary structure and biological function. Likewise, chimeric genes comprising portions of Notch fused to any heterologous (non-Notch) protein-encoding sequences may be constructed. A specific embodiment relates to a chimeric protein comprising a fragment of Notch of at least six amino acids.

In another specific embodiment, the Notch derivative is a fragment of Notch comprising a region of homology with another toporythmic protein. As used herein, a region of a first protein shall be considered “homologous” to a second protein when the amino acid sequence of the region is at least 30% identical or at least 75% either identical or involving conservative changes, when compared to any sequence in the second protein of an equal number of amino acids as the number contained in the region.

Derivatives of Serrate, Delta, other toporythmic proteins, and the adhesive portions thereof, can be made by methods similar to those described supra.

### 5.9.1. Derivatives of Notch Containing One or More Domains of the Protein

In a specific embodiment, the invention provides Therapeutics that are Notch derivatives and analogs, in particular Notch fragments and derivatives of such fragments, that comprise one or more domains of the Notch protein, including but not limited to the extracellular domain, transmembrane domain, intracellular domain, membrane-associated region, one or more of the EGF-like repeats (ELR) of the Notch protein, the cdc10 repeats, and the Notch/lin-12 repeats. In specific embodiments, the Notch derivative may lack all or a portion of the ELRs, or one or more other regions of the protein.

In a specific embodiment, relating to a Notch protein of a species other than D. melanogaster, preferably human, the fragments comprising specific portions of Notch are those comprising portions in the respective Notch protein most homologous to specific fragments of the Drosophila Notch protein (e.g., ELR 11 and ELR 12).

### 5.9.2. Derivatives of Notch or Other Toporythmic Proteins that Mediate Binding to Toporythmic Protein Domains, and Inhibitors Thereof

The invention also provides Notch fragments, and analogs or derivatives of such fragments, which mediate binding to toporythmic proteins (and thus are termed herein “adhesive”), and nucleic acid sequences encoding the foregoing.

Also included as Therapeutics of the invention are toporythmic (e.g., Delta, Serrate) protein fragments, and analogs or derivatives thereof, which mediate heterotypic binding to Notch (and thus are termed herein “adhesive”), and nucleic acid sequences relating to the foregoing.

Also included as Therapeutics of the invention are inhibitors (e.g., peptide inhibitors) of the foregoing toporythmic protein interactions with Notch.

The ability to bind to a toporythmic protein can be demonstrated by in vitro aggregation assays with cells expressing such a toporythmic protein as well as cells expressing Notch or a Notch derivative (See Section 6). That is, the ability of a protein fragment to bind to a Notch protein can be demonstrated by detecting the ability of the fragment, when expressed on the surface of a first cell, to bind to a Notch protein expressed on the surface of a second cell. Inhibitors of the foregoing interactions can be detected by their ability to inhibit such aggregation in vitro.

The nucleic acid sequences encoding toporythmic proteins or adhesive domains thereof, for use in such assays, can be isolated from human, porcine, bovine, feline, avian, equine, canine, or insect, as well as primate sources and any other species in which homologs of known toporythmic genes can be identified.

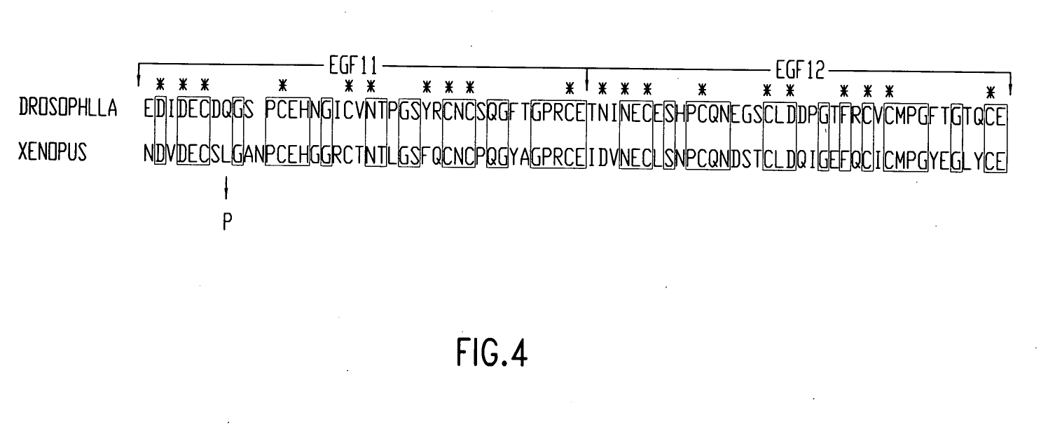

In a specific embodiment, the adhesive fragment of Notch is that comprising the portion of Notch most homologous to ELR 11 and 12, i.e., amino acid numbers 447 through 527 (SEQ ID NO:14) of the Drosophila Notch sequence (see FIG. 4). In yet another specific embodiment, the adhesive fragment of Delta mediating binding to Notch is that comprising the portion of Delta most homologous to about amino acid numbers 1-230 of the Drosophila Delta sequence (SEQ ID NO:2). In a specific embodiment relating to an adhesive fragment of Serrate, such fragment is that comprising the portion of Serrate most homologous to about amino acid numbers 85-283 or 79-282 of the Drosophila Serrate sequence (see FIG. 5 (SEQ ID NO:4)).

Due to the degeneracy of nucleotide coding sequences, other DNA sequences which encode substantially the same amino acid sequence as the adhesive sequences may be used in the practice of the present invention. These include but are not limited to nucleotide sequences comprising all or portions of the Notch, Delta, or Serrate genes which are altered by the substitution of different codons that encode a functionally equivalent amino acid residue within the sequence, thus producing a silent change. Likewise, the adhesive protein fragments or derivatives thereof, of the invention include, but are not limited to, those containing, as a primary amino acid sequence, all or part of the amino acid sequence of the adhesive domains including altered sequences in which functionally equivalent amino acid residues are substituted for residues within the sequence resulting in a silent change.

Adhesive fragments of toporythmic proteins and potential derivatives, analogs or peptides related to adhesive toporythmic protein sequences, can be tested for the desired binding activity e.g., by the in vitro aggregation assays described in the examples herein. Adhesive derivatives or adhesive analogs of adhesive fragments of toporythmic proteins include but are not limited to those peptides which are substantially homologous to the adhesive fragments, or whose encoding nucleic acid is capable of hybridizing to the nucleic acid sequence encoding the adhesive fragments, and which peptides and peptide analogs have positive binding activity e.g., as tested in vitro by an aggregation assay such as described in the examples sections infra. Such derivatives and analogs are envisioned as Therapeutics and are within the scope of the present invention.

The adhesive-protein related derivatives, analogs, and peptides of the invention can be produced by various methods known in the art. The manipulations which result in their production can occur at the gene or protein level (see Section 5.6).

Additionally, the adhesive-encoding nucleic acid sequence can be mutated in vitro or in vivo; and manipulations of the adhesive sequence may also be made at the protein level (see Section 5.6).

In addition, analogs and peptides related to adhesive fragments can be chemically synthesized.

### 5.10. Assays of Notch Proteins, Derivatives and Analogs

The in vitro activity of Notch proteins, derivatives and analogs, and other toporythmic proteins which bind to Notch, can be assayed by various methods.

For example, in one embodiment, where one is assaying for the ability to bind or compete with wild-type Notch for binding to anti-Notch antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention.

In another embodiment, where one is assaying for the ability to mediate binding to Notch, one can carry out an in vitro aggregation assay such as described infra in Section 6 or 7 (see also Fehon et al., 1990, Cell 61:523-534; Rebay et al., 1991, Cell 67:687-699).

In another embodiment, where another ligand for Notch is identified, ligand binding can be assayed, e.g., by binding assays well known in the art. In another embodiment, physiological correlates of ligand binding to cells expressing a Notch receptor (signal transduction) can be assayed.

In another embodiment, in insect or other model systems, genetic studies can be done to study the phenotypic effect of a Notch mutant that is a derivative or analog of wild-type Notch.

Other methods will be known to the skilled artisan and are within the scope of the invention.

### 5.11. Antibodies to Notch Proteins, Derivatives and Analogs

According to one embodiment of the invention, antibodies and fragments containing the binding domain thereof, directed against Notch are Therapeutics. Accordingly, Notch proteins, fragments or analogs or derivatives thereof, in particular, human Notch proteins or fragments thereof, may be used as immunogens to generate anti-Notch protein antibodies. Such antibodies can be polyclonal, monoclonal, chimeric, single chain, Fab fragments, or from an Fab expression library. In a specific embodiment, antibodies specific to EGF-like repeats 11 and 12 of Notch may be prepared. In other embodiments, antibodies reactive with the extracellular domain of Notch can be generated. One example of such antibodies may prevent aggregation in an in vitro assay. In another embodiment, antibodies specific to human Notch are produced.

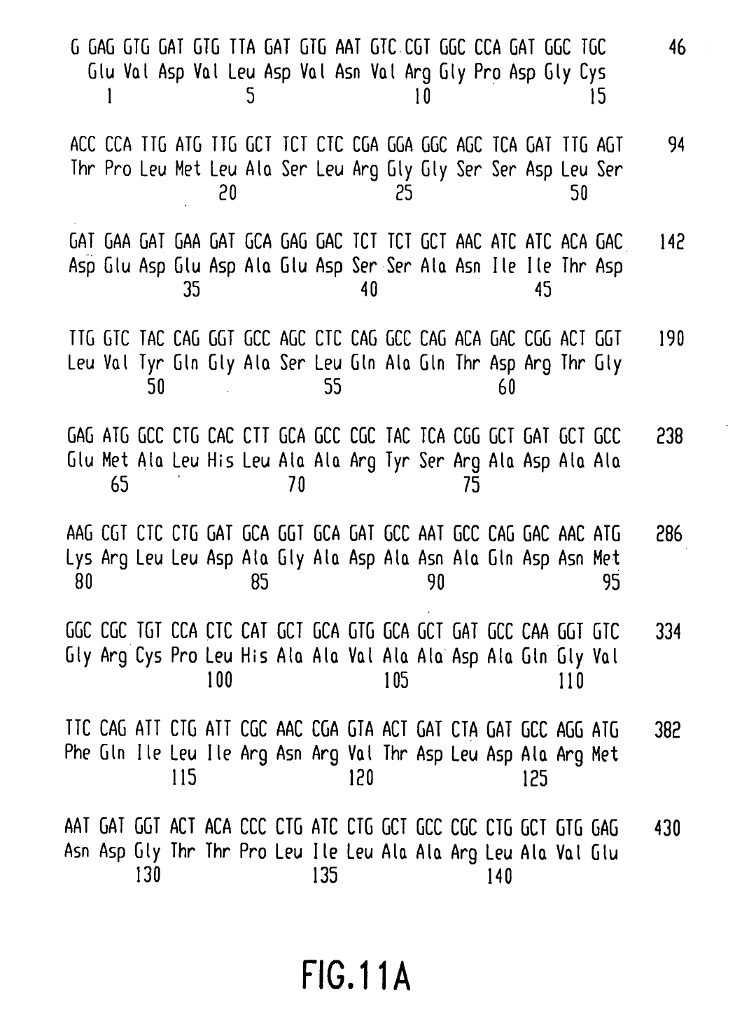

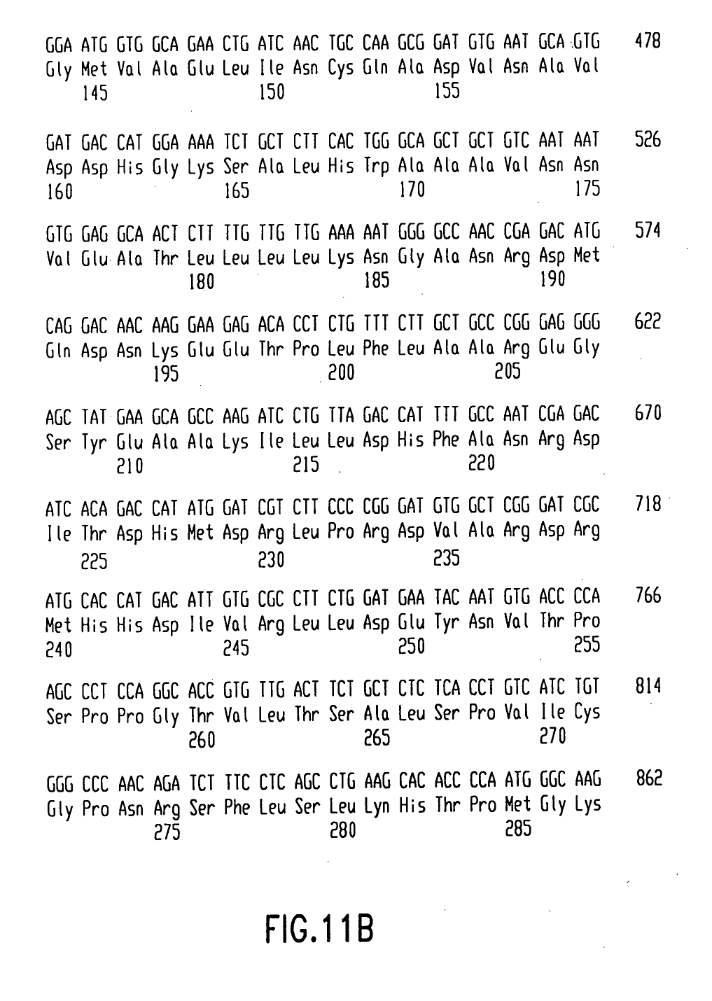

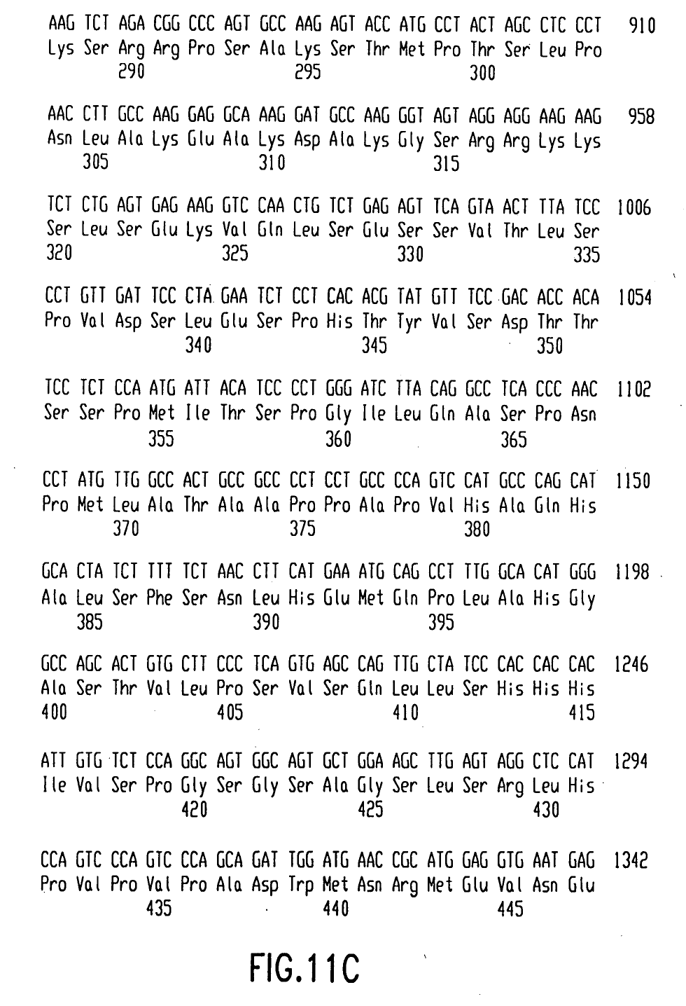

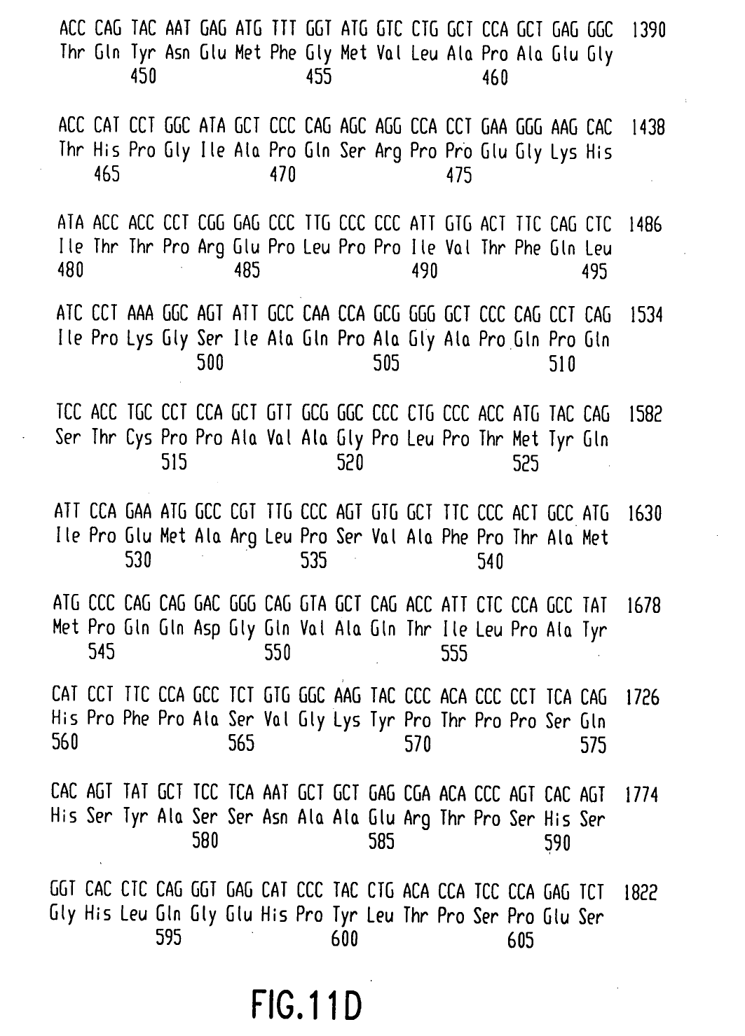

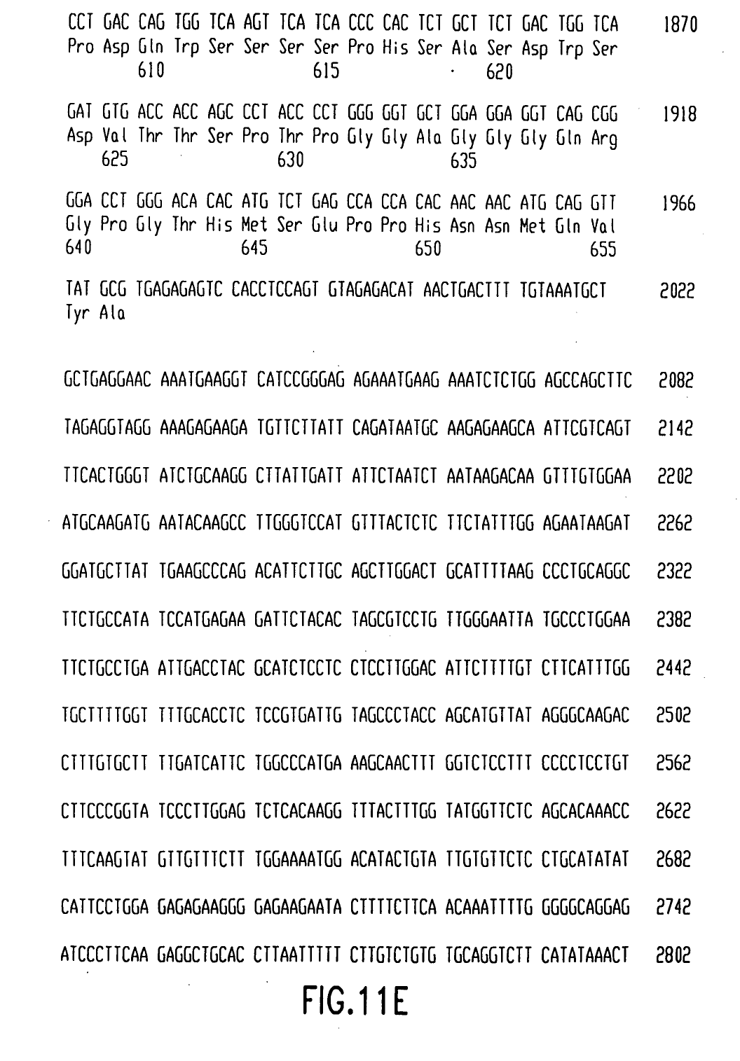

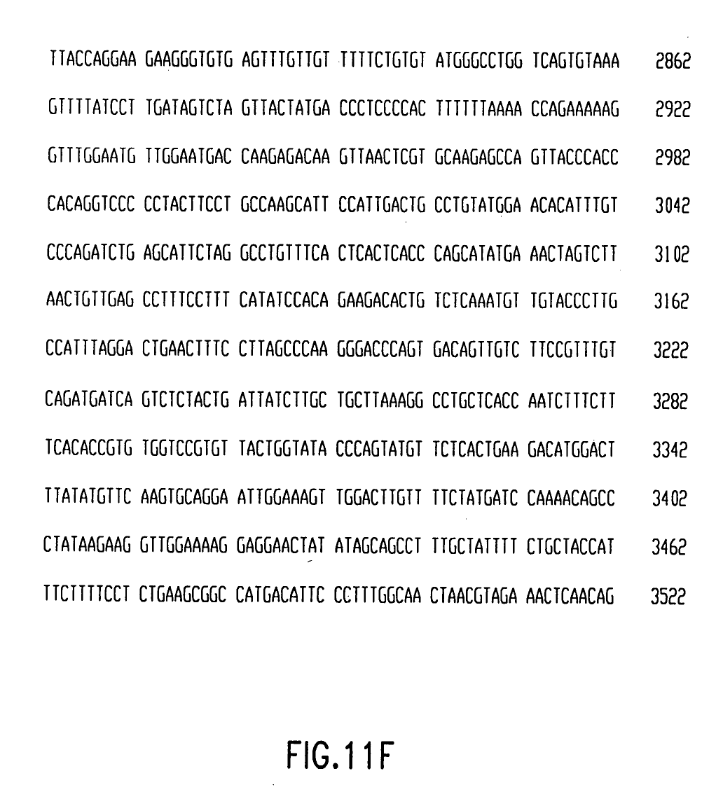

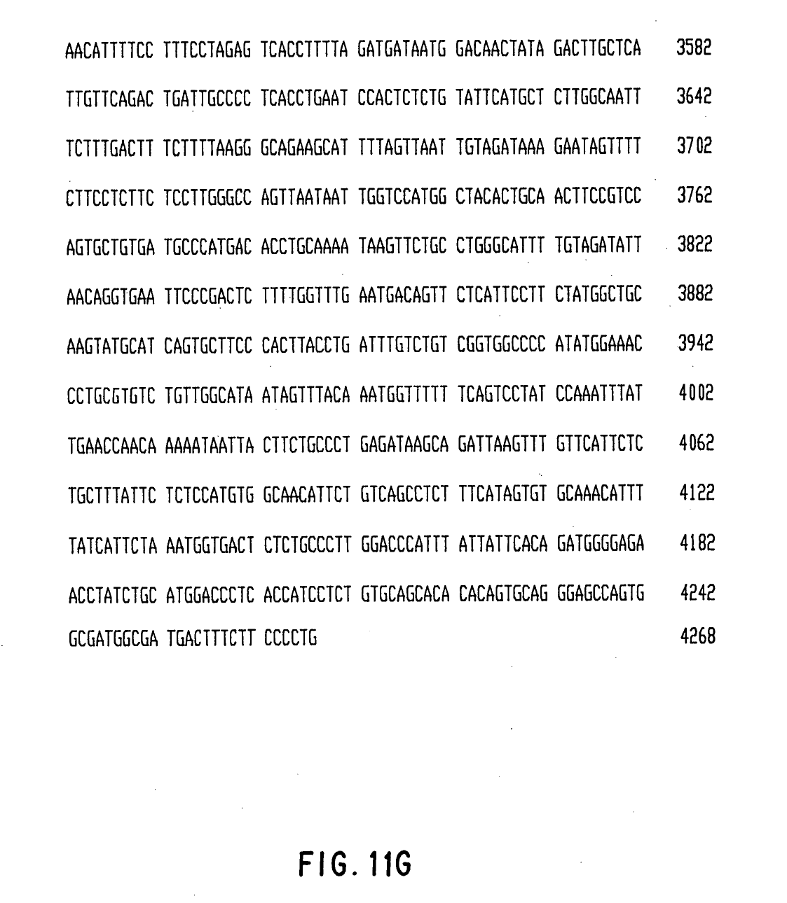

Various procedures known in the art may be used for the production of polyclonal antibodies to a Notch protein or peptide. In a particular embodiment, rabbit polyclonal antibodies to an epitope of the human Notch protein encoded by a sequence depicted in FIG. 10 or 11, or a subsequence thereof, can be obtained. For the production of antibody, various host animals can be immunized by injection with the native Notch protein, or a synthetic version, or fragment thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhold limpet hemocyanins, dinitrophenol, and potentially useful human adjuvants such as BCG (bacille Calmette-Guerin) and corynebacterium parvum.

For preparation of monoclonal antibodies directed toward a Notch protein sequence, any technique which provides for the production of antibody molecules by continuous cell lines in culture may be used. For example, the hybridoma technique originally developed by Kohler and Milstein (1975, Nature 256, 495497), as well as the trioma technique, the human B-cell hybridoma technique (Kozbor et al., 1983, Immunology Today 4, 72), and the EBV-hybridoma technique to produce human monoclonal antibodies (Cole et al., 1985, in Monoclonal Antibodies and Cancer Therapy, Alan R. Liss, Inc., pp. 77-96).

Antibody fragments which contain the idiotype (binding domain) of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g. ELISA (enzyme-linked immunosorbent assay). For example, to select antibodies which recognize the adhesive domain of a Notch protein, one may assay generated hybridomas for a product which binds to a protein fragment containing such domain. For selection of an antibody specific to human Notch, one can select on the basis of positive binding to human Notch and a lack of binding to Drosophila Notch.

In addition to therapeutic utility, the foregoing antibodies have utility in diagnostic immunoassays as described in Section 5.6 supra.

Similar procedures to those described supra can be used to make Therapeutics which are antibodies to domains of other proteins (particularly toporythmic proteins) that bind or otherwise interact with Notch (e.g., adhesive fragments of Delta or Serrate).

### 6. Domains of Notch Mediate Binding with Delta

Intermolecular association between the products of the Notch and Delta genes was detected by studying the effects of their expression on aggregation in Drosophila Schneider's 2 (S2) cells (Fehon et al., 1990, Cell 61, 523-534). Direct evidence of intermolecular interactions between Notch and Delta is described herein, as well as an assay system that can be used in dissecting the components of this interaction. Normally nonadhesive Drosophila S2 cultured cells that express Notch bind specifically in a calcium-dependent manner to cells that express Delta. Furthermore, while cells that express Notch do not bind to one another, cells that express Delta do bind to one another, suggesting that Notch and Delta can compete for binding to Delta at the cell surface. Notch and Delta form detergent-soluble complexes both in cultured cells and embryonic cells, suggesting that Notch and Delta interact directly at the molecular level in vitro and in vivo. The analyses suggest that Notch and Delta proteins interact at the cell surface via their extracellular domains.

### 6.1. Experimental Procedures

### 6.1.1. Expression Constructs

Expression constructs are described in Fehon et al., 1990, Cell 61:523-534. Briefly, Notch encoded by the MgIIa minigene a cDNA/genomic chimeric construct (Ramos et al., 1989, Genetics 123, 337-348) was expressed following insertion into pRmHa-3 (Bunch, et al., 1988, Nucl. Acids Res. 16, 1043-1061). In the resulting construct, designated pMtNMg, the metallothionein promoter in pRmHa-3 is fused to Notch sequences starting 20 nucleotides upstream of the translation start site.

The extracellular Notch construct (ECN1), was derived from a Notch cosmid (Ramos et al., 1989, Genetics 123, 337-348), and has an internal deletion of the Notch coding sequences from amino acids 1790 to 2625 inclusive (Wharton et al., 1985, Cell 43, 567-581), and a predicted frameshift that produces a novel 59 amino acid carboxyl terminus.

For the Delta expression construct, the Dl1 cDNA (Kopczynski et al., 1988, Genes Dev. 2, 1723-1735; FIG. 1; SEQ ID NO:1), which includes the complete coding capacity for Delta, was inserted into the EcoRI site of pRmHa-3. This construct was called pMTDl1.

### 6.1.2. Antibody Preparation

Hybridoma cell line C17.9C6 was obtained from a mouse immunized with a fusion protein based on a 2.1 kb SalI-HindIII fragment that includes coding sequences for most of the intracellular domain of Notch (amino acids 1791-2504; Wharton et al., 1985, Cell 43, 567-581). The fragment was subcloned into pUR289 (Ruther and Muller-Hill, 1983, EMBO J. 2, 1791-1794), and then transferred into the pATH 1 expression vector (Dieckmann and Tzagoloff, 1985, J. Biol. Chem. 260, 1513-1520) as a BglII-HindIII fragment. Soluble fusion protein was expressed, precipitated by 25% (NH₄)₂SO₄, resuspended in 6 M urea, and purified by preparative isoelectric focusing using a Rotofor (Bio-Rad) (for details, see Fehon, 1989, Rotofor Review No. 7, Bulletin 1518, Richmond, Calif.: Bio-Rad Laboratories).

Mouse polyclonal antisera were raised against the extracellular domain of Notch using four BstYl fragments of 0.8 kb (amino acids 237-501: Wharton et al., 1985, Cell 43, 567-581), 1.1 kb (amino acids 501-868), 0.99 kb (amino acids 868-1200), and 1.4 kb (amino acids 1465-1935) length, which spanned from the fifth EGF-like repeat across the transmembrane domain, singly inserted in-frame into the appropriate pGEX expression vector (Smith and Johnson, 1988, Gene 67, 31-40). Fusion proteins were purified on glutathione-agarose beads (SIGMA). Mouse and rat antisera were precipitated with 50% (NH₄)₂SO₄ and resuspended in PBS (150 mM NaCl, 14 mM Na₂HPO₄, 6 mM NaH₂PO₄) with 0.02% NaN₃.

Hybridoma cell line 201 was obtained from a mouse immunized with a fusion protein that includes coding sequences from the extracellular domain of Delta (Kopczynski et al., 1988, Genes Dev. 2, 1723-1735), including sequences extending from the fourth through the ninth EGF-like repeats in Delta (amino acids 350-529).

Rat polyclonal antisera were obtained following immunization with antigen derived from the same fusion protein construct. In this case, fusion protein was prepared by lysis of IPTG-induced cells in SDS-Laemmli buffer (Carroll and Laughon, 1987, in DNA Cloning, Volume III, D. M. Glover, ed. (Oxford: IRL Press), pp. 89-111), separation of proteins by SDS-PAGE, excision of the appropriate band from the gel, and electroelution of antigen from the gel slice for use in immunization (Harlow and Lane, 1988, Antibodies: A Laboratory Manual (Cold Spring Harbor, N.Y.: Cold Spring Harbor Laboratory)).

### 6.1.3. Cell Culture and Transfection

The S2 cell line (Schneider, 1972, J. Embryol. Exp. Morph. 27, 353-365) was grown in M3 medium (prepared by Hazleton Co.) supplemented with 2.5 mg/ml Bacto-Peptone (Difco), 1 mg/ml TC Yeastolate (Difco), 11% heat-inactivated fetal calf serum (FCS) (Hyclone), and 100 U/ml penicillin-100 μg/ml streptomycin-0.25 μg/ml fungizone (Hazleton). Cells growing in log phase at ˜2×10⁶ cells/ml were transfected with 20 μg of DNA-calcium phosphate coprecipitate in 1 ml per 5 ml of culture as previously described (Wigler et al., 1979, Proc. Natl. Acad. Sci. USA 78, 1373-1376), with the exception that BES buffer (SIGMA) was used in place of HEPES buffer (Chen and Okayama, 1987, Mol. Cell. Biol. 7, 2745-2752). After 16-18 hr, cells were transferred to conical centrifuge tubes, pelleted in a clinical centrifuge at full speed for 30 seconds, rinsed once with ¼ volume of fresh complete medium, resuspended in their original volume of complete medium, and returned to the original flask. Transfected cells were then allowed to recover for 24 hr before induction.

### 6.1.4. Aggregation Assays

Expression of the Notch and Delta metallothionein constructs was induced by the addition of CuSO₄ to 0.7 mM. Cells transfected with the ECN1 construct were treated similarly. Cells were then mixed, incubated under aggregation conditions, and scored for their ability to aggregate using specific antisera and immunofluorescence microscopy to visualize expressing cells.

Two types of aggregation assays were used. In the first assay, a total of 3 ml of cells (5-10×10⁶ cells/ml) was placed in a 25 ml Erlenmeyer flask and rotated at 40-50 rpm on a rotary shaker for 24-48 hr at room temperature. For these experiments, cells were mixed 1-4 hr after induction began and induction was continued throughout the aggregation period. In the second assay, ˜0.6 ml of cells were placed in a 0.6 ml Eppendorf tube (leaving a small bubble) after an overnight induction (12-16 hr) at room temperature and rocked gently for 1-2 hr at 4° C. The antibody inhibition and Ca²⁺ dependence experiments were performed using the latter assay. For Ca²⁺ dependence experiments, cells were first collected and rinsed in balanced saline solution (BSS) with 11% FCS (BSS-FCS; FCS was dialyzed against 0.9% NaCl, 5 mM Tris [pH 7.5]) or in Ca²⁺ free BSS-FCS containing 10 mM EGTA (Snow et al., 1989, Cell 59, 313-323) and then resuspended in the same medium at the original volume. For the antibody inhibition experiments, Notch-transfected cells were collected and rinsed in M3 medium and then treated before aggregation in M3 medium for 1 hr at 4° C. with a 1:250 dilution of immune or preimmune sera from each of the four mice immunized with fusion proteins containing segments from the extracellular domain of Notch (see Antibody Preparation above).

### 6.1.5. Immunofluorescence

Cells were collected by centrifugation (3000 rpm for 20 seconds in an Eppendorf microcentrifuge) and fixed in 0.6 ml Eppendorf tubes with 0.5 ml of freshly made 2% paraformaldehyde in PBS for 10 min at room temperature. After fixing, cells were collected by centrifugation, rinsed twice in PBS, and stained for 1 hr in primary antibody in PBS with 0.1% saponin (SIGMA) and 1% normal goat serum (Pocono Rabbit Farm, Canadensis, Pa.). Monoclonal antibody supernatants were diluted 1:10 and mouse or rat sera were diluted 1:1000 for this step. Cells were then rinsed once in PBS and stained for 1 hr in specific secondary antibodies (double-labeling grade goat anti-mouse and goat anti-rat, Jackson Immunoresearch) in PBS-saponin-normal goat serum. After this incubation, cells were rinsed twice in PBS and mounted on slides in 90% glycerol, 10% 1 M Tris (pH 8.0), and 0.5% n-propyl gallate. Cells were viewed under epifluorescence on a Leitz Orthoplan 2 microscope.

Confocal micrographs were taken using the Bio-Rad MRC 500 system connected to a Zeiss Axiovert compound microscope. Images were collected using the BHS and GHS filter sets, aligned using the ALIGN program, and merged using MERGE. Fluorescent bleed-through from the green into the red channel was reduced using the BLEED program (all software provided by Bio-Rad). Photographs were obtained directly from the computer monitor using Kodak Ektar 125 film.

### 6.1.6. Cell Lysates, Immunoprecipitations, and Western Blots

Nondenaturing detergent lysates of tissue culture and wild-type Canton-S embryos were prepared on ice in ˜10 cell vol of lysis buffer (300 mM NaCl, 50 mM Tris [pH 8.0], 0.5% NP-40, 0.5% deoxycholate, 1 mM CaCl₂, 1 mM MgCl₂) with 1 mM phenylmethysulfonyl fluoride (PMSF) and diisopropyl fluorophosphate diluted 1:2500 as protease inhibitors. Lysates were sequentially triturated using 18G, 21G, and 25G needles attached to 1 cc tuberculin syringes and then centrifuged at full speed in a microfuge 10 min at 4° C. to remove insoluble material. Immunoprecipitation was performed by adding ˜1 μg of antibody (1-2 μl of polyclonal antiserum) to 250-500 μl of cell lysate and incubating for 1 hr at 4° C. with agitation. To this mixture, 15 μg of goat anti-mouse antibodies (Jackson Immunoresearch; these antibodies recognize both mouse and rat IgG) were added and allowed to incubate for 1 hr at 4° C. with agitation. This was followed by the addition of 100 μl of fixed Staphylococcus aureus (Staph A) bacteria (Zysorbin, Zymed; resuspended according to manufacturer's instructions), which had been collected, washed five times in lysis buffer, and incubated for another hour. Staph A-antibody complexes were then pelleted by centrifugation and washed three times in lysis buffer followed by two 15 min washes in lysis buffer. After being transferred to a new tube, precipitated material was suspended in 50 μl of SDS-PAGE sample buffer, boiled immediately for 10 min, run on 3%-15% gradient gels, blotted to nitrocellulose, and detected using monoclonal antibodies and HRP-conjugated goat anti-mouse secondary antibodies as previously described (Johansen et al., 1989, J. Cell Biol. 109, 2427-2440). For total cellular protein samples used on Western blots, cells were collected by centrifugation, lysed in 10 cell vol of sample buffer that contained 1 mM PMSF, and boiled immediately.

### 6.2. Results

### 6.2.1. The Expression of Notch and Delta in Cultured Cells

To detect interactions between Notch and Delta, we examined the behavior of cells expressing these proteins on their surfaces using an aggregation assay. We chose the S2 cell line (Schneider, 1972, J. Embryol. Exp. Morph. 27, 353-365) for these studies. S2 cells express an aberrant Notch message and no detectable Notch due to a rearrangement of the 5′ end of the Notch coding sequence. These cells also express no detectable Delta.

Results of Western blot and immunofluorescentanalysis clearly showed that the Notch and Delta constructs support expression of proteins of the expected sizes and subcellular localization.

### 6.2.2. Cells that Express Notch and Delta Aggregate

A simple aggregation assay was used to detect interactions between Notch and Delta expressed on the surface of S2 cells.

S2 cells in log phase growth were separately transfected with either the Notch or Delta metallothionein promoter construct. After induction with CuSO₄, transfected cells were mixed in equal numbers and allowed to aggregate overnight at room temperature (for details, see Experimental Procedures, Section 6.1). Alternatively, in some experiments intended to reduce metabolic activity, cells were mixed gently at 4° C. for 1-2 hr. To determine whether aggregates had formed, cells were processed for immunofluorescence microscopy using antibodies specific for each gene product and differently labeled fluorescent secondary antibodies. Expressing cells usually constituted less than 5% of the total cell population because transient rather than stable transformants were used. The remaining cells either did not express a given protein or expressed at levels too low for detection by immunofluorescence microscopy. As controls, we performed aggregations with only a single type of transfected cell.

The results (Fehon et al., 1990, Cell 61:523-534) showed that while Notch-expressing (Notch⁺) cells alone did not form aggregates in the assay, Delta-expressing (Delta⁺) cells did. The tendency for Delta⁺ cells to aggregate was apparent even in nonaggregated control samples, where cell clusters of 4-8 cells that probably arose from adherence between mitotic sister cells commonly occurred. However, clusters were more common after incubation under aggregation conditions (e.g., 19% of Delta⁺ cells in aggregates before incubation vs. 37% of Delta⁺ cells in aggregates after incubation), indicating that Delta⁺ cells are able to form stable contacts with one another in this assay.

In remarkable contrast to control experiments with Notch⁺ cells alone, aggregation of mixtures of Notch⁺ and Delta⁺ cells resulted in the formation of clusters of up to 20 or more cells. The fraction of expressing cells found in clusters of four or more stained cells after 24 hr of aggregation ranged from 32%-54% in mixtures of Notch⁺ and Delta⁺ cells. This range was similar to that seen for Delta⁺ cells alone (37%-40%) but very different from that for Notch⁺ cells alone (only 0%-5%). Although a few clusters that consisted only of Delta⁺ cells were found, Notch⁺ cells were never found in clusters of greater than four to five cells unless Delta⁺ cells were also present. Again, all cells within these clusters expressed either Notch or Delta, even though transfected cells composed only a small fraction of the total cell population. At 48 hr, the degree of aggregation appeared higher (63%-71%), suggesting that aggregation had not yet reached a maximum after 24 hr under these conditions. Also, cells cotransfected with Notch and Delta constructs (so that all transfected cells express both proteins) aggregated in a similar fashion under the same experimental conditions.

Notch involvement in the aggregation process was directly tested by examining the effect of a mixture of polyclonal antisera directed against fusion proteins that spanned almost the entire extracellular domain of Notch on aggregation (see Experimental Procedures, Section 6.1). To minimize artifacts that might arise due to a metabolic response to patching of surface antigens, antibody treatment and the aggregation assay were performed at 4° C. in these experiments. Notch⁺ cells were incubated with either preimmune or immune mouse sera for 1 hr, Delta⁺ cells were added, and aggregation was performed for 1-2 hr. While Notch⁺ cells pretreated with preimmune sera aggregated with Delta⁺ cells (in one of three experiments, 23% of the Notch⁺ cells were in Notch⁺-Delta⁺ cell aggregates), those treated with immune sera did not (only 2% of Notch⁺ cells were in aggregates). This result suggested that the extracellular domain of Notch was required for Notch⁺-Delta⁺ cell aggregation.

### 6.2.3. Notch-Delta-Mediated Aggregation is Calcium Dependent

The ability of expressing cells to aggregate in the presence or absence of Ca²⁺ ions was tested to determine whether there is a Ca²⁺ ion requirement for Notch-Delta aggregation. To minimize possible nonspecific effects due to metabolic responses to the removal of Ca²⁺, these experiments were performed at 4° C. The results clearly demonstrated a dependence of Notch-Delta-mediated aggregation on exogenous Ca²⁺.

### 6.2.4. Notch and Delta Interact within a Single Cell

The question whether Notch and Delta are associated within the membrane of one cell that expresses both proteins was posed by examining the distributions of Notch and Delta in cotransfected cells. To test whether the observed colocalization was coincidental or represented a stable interaction between Notch and Delta, live cells were treated with an excess of polyclonal anti-Notch antiserum. This treatment resulted in “patching” of Notch on the surface of expressing cells into discrete patches as detected by immunofluorescence. There was a distinct correlation between the distributions of Notch and Delta on the surfaces of these cells after this treatment, indicating that these proteins are associated within the membrane.

### 6.2.5. Interactions with Delta do not Require the Intracellular Domain of Notch

In addition to a large extracellular domain that contains EGF-like repeats, Notch has a sizeable intracellular (IC) domain of ˜940 amino acids. The IC domain includes a phosphorylation site (Kidd et al., 1989, Genes Dev. 3, 1113-1129), a putative nucleotide binding domain, a polyglutamine stretch (Wharton et al., 1985, Cell 43, 567-581; Kidd, et al., 1986, Mol. Cell. Biol. 6, 3094-3108), and sequences homologous to the yeast cdc10 gene, which is involved in cell cycle control in yeast (Breeden and Nasmyth, 1987, Nature 329, 651-654). A variant Notch construct was used from which coding sequences for ˜835 amino acids of the IC domain, including all of the structural features noted above, had been deleted (leaving 25 membrane-proximal amino acids and a novel 59 amino acid carboxyl terminus; see Experimental Procedures).

In aggregation assays, cells that expressed the ECN1 construct consistently formed aggregates with Delta⁺ cells, but not with themselves, just as was observed for cells that expressed intact Notch. Sharp bands of ECN1 staining were observed within regions of contact with Delta⁺ cells, again indicating a localization of ECN1 within regions of contact between cells. To test for interactions within the membrane, surface antigen co-patching experiments were conducted using cells cotransfected with the ECN1 and Delta constructs. As observed for intact Notch, when ECN1 was patched using polyclonal antisera against the extracellular domain of Notch, ECN1 and Delta colocalized at the cell surface. These results demonstrate that the observed interactions between Notch and Delta within the membrane do not require the deleted portion of the IC domain of Notch and are therefore probably mediated by the extracellular domain.

### 6.2.6. Notch and Delta Form Detergent-Soluble Intermolecular Complexes

The preceding results indicated molecular interactions between Notch and Delta present within the same membrane and between these proteins expressed on different cells. A further test for such interactions is whether these proteins would coprecipitate from nondenaturing detergent extracts of cells that express Notch and Delta. If Notch and Delta form a stable intermolecular complex either between or within cells, then it should be possible to precipitate both proteins from cell extracts using specific antisera directed against one of these proteins. This analysis was performed by immunoprecipitating Delta with polyclonal antisera from NP40/deoxycholate lysates (see Experimental Procedures) of cells cotransfected with the Notch and Delta constructs that had been allowed to aggregate overnight or of 0-24 hr wild-type embryos.

Coprecipitation of Notch was detected in Delta immunoprecipitates from cotransfected cells and embryos. However, coprecipitating Notch appeared to be present in much smaller quantities than Delta and was therefore difficult to detect. The fact that immunoprecipitation of Delta results in the coprecipitation of Notch constitutes direct evidence that these two proteins form stable intermolecular complexes in transfected S2 cells and in embryonic cells.

### 6.3. Discussion

Use of an in vitro aggregation assay that employs normally nonadhesive S2 cells showed that cells that express Notch and Delta adhere specifically to one another.

### 7. EGF Repeats 11 and 12 of Notch are Required and Sufficient for Notch-Delta-Mediated Aggregation

The same aggregation assay was used as described in Section 6, together with deletion mutants of Notch to identify regions within the extracellular domain of Notch necessary for interactions with Delta. The evidence shows that the EGF repeats of Notch are directly involved in this interaction and that only two (ELR 11 and 12) of the 36 EGF repeats appear necessary. These two EGF repeats are sufficient for binding to Delta and that the calcium dependence of Notch-Delta mediated aggregation also associates with these two repeats. Finally, the two corresponding EGF repeats from the Xenopus homolog of Notch also mediate aggregation with Delta, implying that not only has the structure of Notch been evolutionarily conserved, but also its function. These results suggest that the extracellular domain of Notch is surprisingly modular, and could potentially bind a variety of proteins in addition to Delta. (See Rebay et al., 1991, Cell 67:687-699.)

### 7.1. Experimental Procedures

### 7.1.1. Expression Constructs

The constructs described are all derivatives of the full length Notch expression construct #1 pMtNMg (see Section 6, supra), and were made as described (Rebay et al., 1991, Cell 67:687-699).

### 7.1.2. Cell Culture and Transfection

The Drosophila S2 cell line was grown and transfected as described in Section 6, supra. The Delta-expressing stably transformed S2 cell line L-49-6-7 (kindly established by L. Cherbas) was grown in M3 medium (prepared by Hazleton Co.) supplemented with 11% heat inactivated fetal calf serum (FCS) (Hyclone), 100 U/ml penicillin-100 μg/ml streptomycin-0.25 μg/ml fungizone (Hazleton), 2×10⁻⁷ M methotrexate, 0.1 mM hypoxanthine, and 0.016 mM thymidine.

### 7.1.3. Aggregation Assays and Immunofluorescence

Aggregation assays and Ca⁺⁺ dependence experiments were as described supra, Section 6. Cells were stained with the anti-Notch monoclonal antibody 9C6.C17 and anti-Delta rat polyclonal antisera (details described in Section 6, supra). Surface expression of Notch constructs in unpermeabilized cells was assayed using rat polyclonal antisera raised against the 0.8 kb (amino acids 237-501; Wharton et al., 1985, Cell 43, 567-581) BstYI fragment from the extracellular domain of Notch. Cells were viewed under epifluorescence on a Leitz Orthoplan 2 microscope.

### 7.2. Results

### 7.2.1. EGF Repeats 11 AND 12 of Notch are Required for Notch-Delta Mediated Aggregation

An extensive deletion analysis was undertaken of the extracellular domain of the Notch protein, which was shown (supra, Section 6; Fehon et al., 1990, Cell 61:523-534) to be involved in Notch-Delta interactions, to identify the precise domain of Notch mediating these interactions. The ability of cells transfected with the various deletion constructs to interact with Delta was tested using the aggregation assay described in Section 6. Briefly, Notch deletion constructs were transiently transfected into Drosophila S2 cells, induced with CuSO₄, and then aggregated overnight at room temperature with a small amount of cells from the stably transformed Delta expressing cell line LA9-6-7(Cherbas), yielding a population typically composed of 1% Notch expressing cells and ˜5% Delta expressing cells, with the remaining cells expressing neither protein.

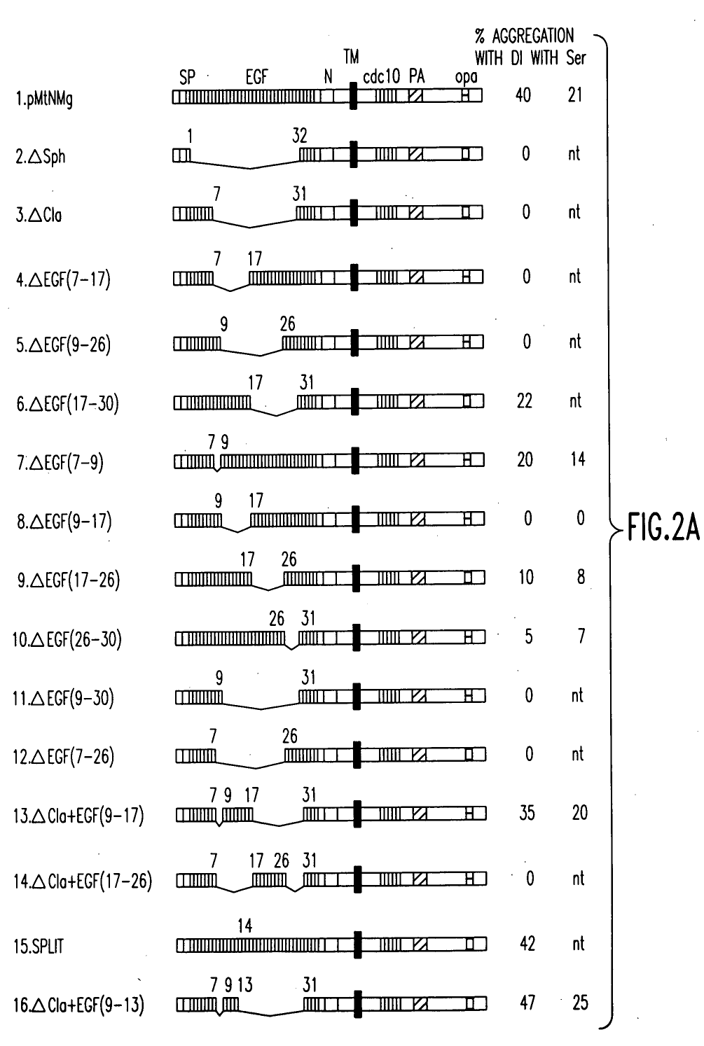

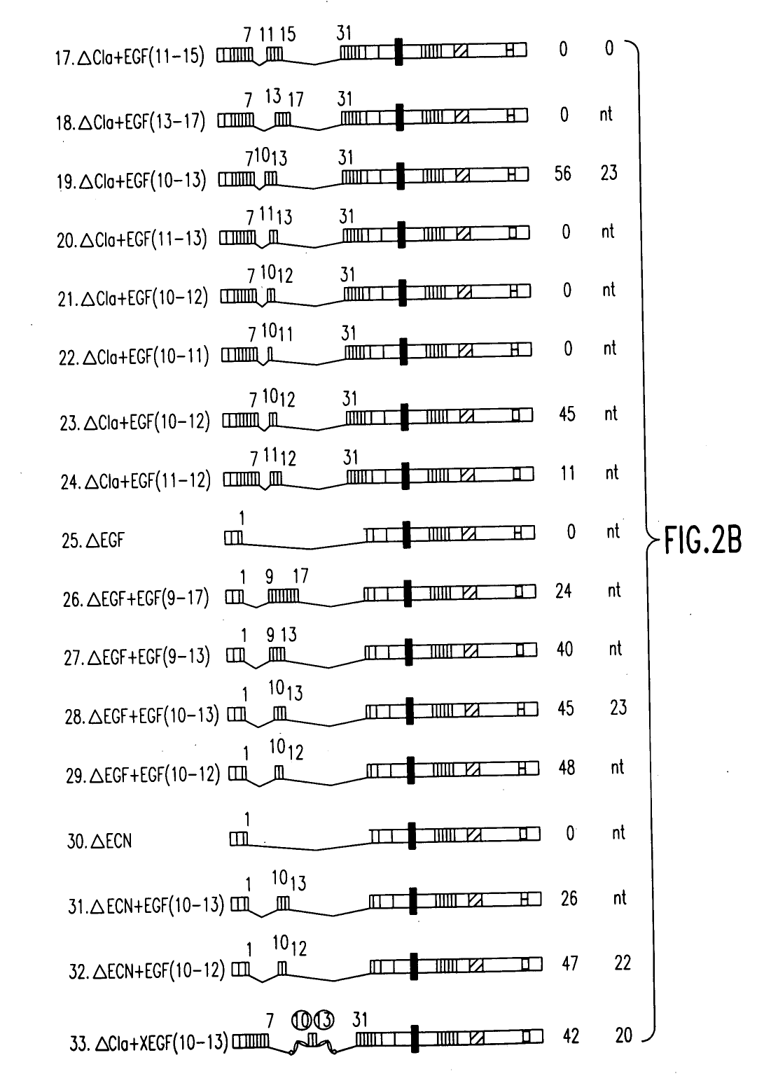

Schematic drawings of the constructs tested and results of the aggregation experiments are shown in FIG. 2. To assay the degree of aggregation, cells were stained with antisera specific to each gene product and examined with immunofluorescent microscopy. Aggregates were defined as clusters of four or more cells containing both Notch and Delta expressing cells, and the values shown in FIG. 2 represent the percentage of all Notch expressing cells found in such clusters. All numbers reflect the average result from at least two separate transfection experiments in which at least 100 Notch expressing cell units (either single cells or clusters) were scored.

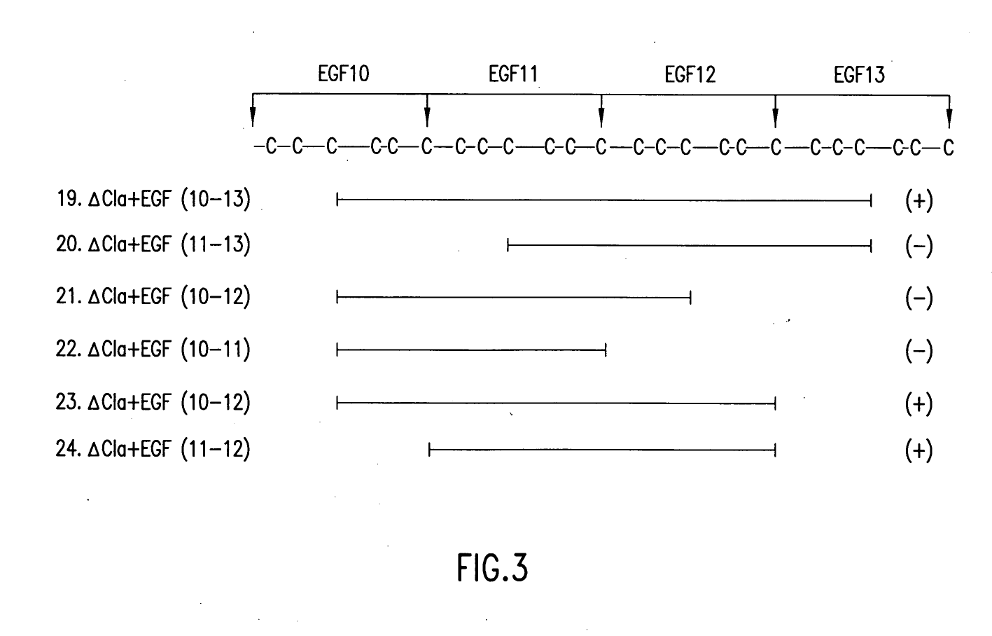

The initial constructs (#2 DSph and #3 ΔCla) deleted large portions of the EGF repeats. Their inability to promote Notch-Delta aggregation suggested that the EGF repeats of Notch were involved in the interaction with Delta. A series of six in-frame ClaI restriction sites was used to further dissect the region between EGF repeats 7 and 30. Due to sequence homology between repeats, five of the ClaI sites occur in the same relative place within the EGF repeat, just after the third cysteine, while the sixth site occurs just before the first cysteine of EGF repeat 31 (FIG. 3). Thus, by performing a partial ClaI digestion and then religating, deletions were obtained that not only preserved the open reading frame of the Notch protein but in addition frequently maintained the structural integrity and conserved spacing, at least theoretically, of the three disulfide bonds in the chimeric EGF repeats produced by the religation (FIG. 2, constructs #4-14). Unfortunately, the most 3′ClaI site was resistant to digestion while the next most 3′ClaI site broke between EGF repeats 30 and 31. Therefore, when various ClaI digestion fragments were reinserted into the framework of the complete ClaI digest (construct #3 ΔCla), the overall structure of the EGF repeats was apparently interrupted at the 3′ junction.

Several points about this series of constructs are worth noting. First, removal of the ClaI restriction fragment breaking in EGF repeats 9 and 17 (construct #8 ΔEGF9-17) abolished aggregation with Delta, while reinsertion of this piece into construct #3 ΔCla, which lacks EGF repeats 7-30, restored aggregation to roughly wild type levels (construct #13 ΔCla+EGF9-17), suggesting that EGF repeats 9 through 17 contain sequences important for binding to Delta. Second, all constructs in this series (#4-14) were consistent with the binding site mapping to EGF repeats 9 through 17. Expression constructs containing these repeats (#6, 7, 9, 10, 13) promoted Notch-Delta interactions while constructs lacking these repeats (#4, 5, 8, 11, 12, 14) did not. To confirm that inability to aggregate with Delta cells was not simply due to failure of the mutagenized Notch protein to reach the cell surface, but actually reflected the deletion of the necessary binding site, cell surface expression of all constructs was tested by immunofluorescently staining live transfected cells with antibodies specific to the extracellular domain of Notch. All constructs failing to mediate Notch-Delta interactions produced a protein that appeared to be expressed normally at the cell surface. Third, although the aggregation assay is not quantitative, two constructs which contained EGF repeats 9-17, #9 ΔEGF17-26 or most noticeably #10 ΔEGF26-30, aggregated at a seemingly lower level. Cells transfected with constructs #9 ΔEGF17-26 and 10 ΔEGF26-30 showed considerably less surface staining than normal, although fixed and permeabilized cells reacted with the same antibody stained normally, indicating the epitopes recognized by the antisera had not been simply deleted. By comparing the percentage of transfected cells in either permeabilized or live cell populations, it was found that roughly 50% of transfected cells for construct #9 ΔEGF17-26 and 10% for construct #10 ΔEGF26-30 produced detectable protein at the cell surface. Thus these two constructs produced proteins which often failed to reach the cell surface, perhaps because of misfolding, thereby reducing, but not abolishing, the ability of transfected cells to aggregate with Delta-expressing cells.

Having mapped the binding site to EGF repeats 9 through 17, further experiments (Rebay et al., 1991, Cell 67:687-699) revealed that EGF repeat 14 of Notch was not involved in the interactions with Delta modelled by the tissue culture assay.

To further map the Delta binding domain within EGF repeats 9-17, specific oligonucleotide primers and the PCR technique were used to generate several subfragments of this region. Three overlapping constructs, #16, 17 and 18 were produced, only one of which, #16 ΔCla+EGF9-13, when transfected into S2 cells, allowed aggregation with Delta cells. Construct #19 ΔCla+EGF(10-13), which lacks EGF repeat 9, further defined EGF repeats 10-13 as the region necessary for Notch-Delta interactions.

Constructs #20-24 represented attempts to break this domain down even further using the same PCR strategy (see FIG. 3). Constructs #20 ΔCla+EGF(11-13), in which EGF repeat 12 is the only entire repeat added, and #21 ΔCla+EGF(10-12), in which EGF repeat 11 is the only entire repeat added, failed to mediate aggregation, suggesting that the presence of either EGF repeat 11 or 12 alone was not sufficient for Notch-Delta interactions. However, since the 3′ ligation juncture of these constructs interrupted the overall structure of the EGF repeats, it was possible that a short “buffer” zone was needed to allow the crucial repeat to function normally. Thus for example in construct #19 ΔCla+EGF(10-13), EGF repeat 12 might not be directly involved in binding to Delta but instead might contribute the minimum amount of buffer sequence needed to protect the structure of EGF repeat 11, thereby allowing interactions with Delta. Constructs #22-24 addressed this issue. Constructs #22 ΔCla+EGF(10-11), which did not mediate aggregation, and #23 ΔCla+EGF(10-12), which did, again suggested that both repeats 11 and 12 are required while the flanking sequence from repeat 13 clearly is not. Finally, construct #24 ΔCla+EGF(11-12), although now potentially structurally disrupted at the 5′ junction, convincingly demonstrated that the sequences from EGF repeat 10 are not crucial. Thus based on entirely consistent data from 24 constructs, EGF repeats 11 and 12 of Notch together define the smallest functional unit obtainable from this analysis that contains the necessary sites for binding to Delta in transfected S2 cells.

### 7.2.2. EGF Repeats 11 AND 12 of Notch are Sufficient for Notch-Delta Mediated Aggregation

The large ClaI deletion into which PCR fragments were inserted (#3 ΔCla) retains roughly ⅓ of the original 36 EGF repeats as well as the three Notch/lin-12 repeats. While these are clearly not sufficient to promote aggregation, it is possible that they form a necessary framework within which specific EGF repeats can interact with Delta. To test whether only a few EGF repeats were in fact sufficient to promote aggregation, two constructs were designed, #25 ΔEGF which deleted all 36 EGF repeats except for the first two-thirds of repeat 1, and #30 ΔECN which deleted the entire extracellular portion of Notch except for the first third of EGF repeat 1 and ˜35 amino acids just before the transmembrane domain. Fragments which had mediated Notch-Delta aggregation in the background of construct #3 ΔCla, when inserted into construct #25 ΔEGF, were again able to promote interactions with Delta (constructs #26-30). Analogous constructs (#31,32) in which the Notch/lin-12 repeats were also absent, again successfully mediated Notch-Delta aggregation. Thus EGF repeats 11 and 12 appear to function as independent modular units which are sufficient to mediate Notch-Delta interactions in S2 cells, even in the absence of most of the extracellular domain of Notch.

### 7.2.3. EGF Repeats 11 and 12 of Notch Maintain the Calcium Dependence of Notch-Delta Mediated Aggregation

The ability of cells expressing certain deletion constructs to aggregate with Delta expressing cells was examined in the presence or absence of Ca⁺⁺ ions. The calcium dependence of the interaction was preserved in even the smallest construct, consistent with the notion that the minimal constructs containing EGF repeats 11 and 12 bind to Delta in a manner similar to that of full length Notch.

### 7.2.4. The Delta Binding Function of EGF Repeats 11 AND 12 of Notch is Conserved in the Xenopus Homolog of Notch

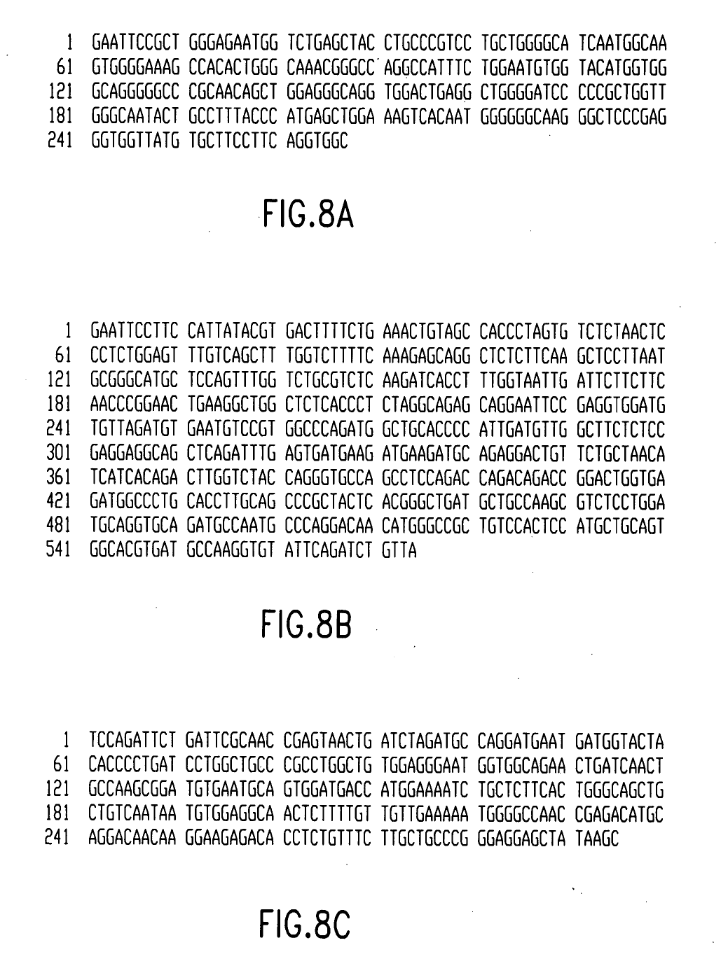

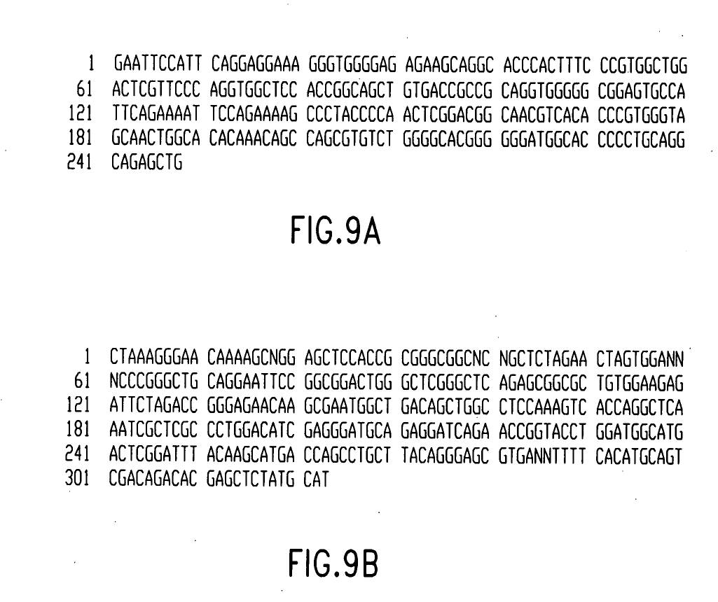

PCR primers based on the Xenopus Notch sequence (Coffman et al., 1990, Science 249, 1438-1441) were used to obtain an ˜350 bp fragment from a Xenopus Stage 17 cDNA library that includes EGF repeats 11 and 12 flanked by half of repeats 10 and 13 on either side. This fragment was cloned into construct #3 ΔCla, and three independent clones were tested for ability to interact with Delta in the cell culture aggregation assay. Two of the clones, #33a&bΔCla+XEGF(10-13), when transfected into S2 cells were able to mediate Notch-Delta interactions at a level roughly equivalent to the analogous Drosophila Notch construct #19ΔCla+EGF(10-13), and again in a calcium dependent manner (Table III). However, the third clone #33cΔCla+XEGF(10-13) failed to mediate Notch-Delta interactions although the protein was expressed normally at the cell surface as judged by staining live unpermeabilized cells. Sequence comparison of the Xenopus PCR product in constructs #33a and 33c revealed a missense mutation resulting in a leucine to proline change (amino acid #453, Coffman, et al., 1990, Science 249, 1438-1441) in EGF repeat 11 of construct #33c. Although this residue is not conserved between Drosophila and Xenopus Notch (FIG. 8), the introduction of a proline residue might easily disrupt the structure of the EGF repeat, and thus prevent it from interacting properly with Delta.

Comparison of the amino acid sequence of EGF repeats 11 and 12 of Drosophila and Xenopus Notch reveals a high degree of amino acid identity, including the calcium binding consensus sequence (FIG. 4, SEQ ID NO:1 and NO:2). However the level of homology is not strikingly different from that shared between most of the other EGF repeats, which overall exhibit about 50% identity at the amino acid level. This one to one correspondence between the individual EGF repeats of Drosophila and Xenopus Notch, together with the functional conservation of ELR 11 and 12, suggests that the 36 EGF repeats of Notch comprise a tandem area of conserved functional units.

### 7.3. Discussion

An extensive deletion analysis of the extracellular domain of Notch was used to show that the regions of Notch containing EGF-homologous repeats 11 and 12 are both necessary and sufficient for Notch-Delta-mediated aggregation, and that this Delta binding capability has been conserved in the same two EGF repeats of Xenopus Notch. The finding that the aggregation mapped to EGF repeats 11 and 12 of Notch demonstrates that the EGF repeats of Notch also function as specific protein binding domains. EGF repeats 11 and 12 alone (#32ΔECN+EGF(11-12)) were sufficient to maintain the Ca⁺⁺ dependence of Notch-Delta interactions.

The various deletion constructs suggest that ELR 11 and ELR 12 function as a modular unit, independent of the immediate context into which they are placed. Thus, neither the remaining 34 EGF repeats nor the three Notch/lin-12 repeats appear necessary to establish a structural framework required for EGF repeats 11 and 12 to function. Interestingly, almost the opposite effect was observed: although the aggregation assay does not measure the strength of the interaction, as the binding site was narrowed down to smaller and smaller fragments, an increase was observed in the ability of the transfected cells to aggregate with Delta expressing cells, suggesting that the normal flanking EGF sequences actually impede association between the proteins. The remaining 34 EGF repeats may also form modular binding domains for other proteins interacting with Notch at various times during development.

The finding that EGF repeats 11 and 12 of Notch form a discrete Delta binding unit represents the first concrete evidence supporting the idea that each EGF repeat or small subset of repeats may play a unique role during development, possibly through direct interactions with other proteins. The homologies seen between the adhesive domain of Delta and Serrate (FIG. 5) suggest that the homologous portion of Serrate is “adhesive” in that it mediates binding to other toporythmic proteins (see Section 8, infra). In addition, the gene scabrous, which encodes a secreted protein with similarity to fibrinogen, may interact with Notch.

In addition to the EGF repeat, multiple copies of other structural motifs commonly occur in a variety of proteins. One relevant example is the cdc10/ankyrin motif, six copies of which are found in the intracellular domain of Notch. Ankyrin contains 22 of these repeats. Perhaps repeated arrays of structural motifs may in general represent a linear assembly of a series of modular protein binding units. Given these results together with the known structural, genetic and developmental complexity of Notch, Notch may interact with a number of different ligands in a precisely regulated temporal and spacial pattern throughout development. Such context specific interactions with extracellular proteins could be mediated by the EGF and Notch/lin-12 repeats, while interactions with cytoskeletal and cytoplasmic proteins could be mediated by the intracellular cdc10/ankyrin motifs.

### 8. Sequences which Mediate Notch-Serrate Interactions

As described herein, the two EGF repeats of Notch which mediate interactions with Delta, namely EGF repeats 11 and 12, also constitute a Serrate binding domain (see Rebay et al., 1991, Cell 67:687-699).

To test whether Notch and Serrate directly interact, S2 cells were transfected with a Serrate expression construct and mixed with Notch expressing cells in an aggregation assay. For the Serrate expression construct, a synthetic primer containing an artificial BamHI site immediately 5′ to the initiator AUG at position 442 (all sequence numbers are according to Fleming et al.; 1990, Genes & Dev. 4:2188-2201) and homologous through position 464, was used in conjunction with a second primer from position 681-698 to generate a DNA fragment of −260 base pairs. This fragment was cut with BamHI and KpnI (position 511) and ligated into Bluescript KS+ (Stratagene). This construct, BTSer5′PCR, was checked by sequencing, then cut with KpnI. The Serrate KpnI fragment (571-2981) was inserted and the proper orientation selected, to generate BTSer5′PCR-Kpn. The 5′ SacII fragment of BTSer5′PCR-Kpn (SacII sites in Bluescript polylinker and in Serrate (1199)) was isolated and used to replace the 5′ SacII fragment of cDNA C1 (Fleming et al., 1990, Genes & Dev. 4:2188-2201), thus regenerating the full length Serrate cDNA minus the 5′ untranslated regions. This insert was isolated by a Sail and partial BamHI digestion and shuttled into the BamHI and SalI sites of pRmHa-3 to generate the final expression construct, Ser-mtn.

Serrate expressing cells adhered to Notch expressing cells in a calcium dependent manner (FIG. 2 and Rebay et al., 1991, supra). However, unlike Delta, under the experimental conditions tested, Serrate did not appear to interact homotypically. In addition, no interactions were detected between Serrate and Delta.

A subset of Notch deletion constructs were tested, and showed that EGF repeats 11 and 12, in addition to binding to Delta, also mediate interactions with Serrate (FIG. 2; Constructs #1, 7-10, 13, 16, 17, 19, 28, and 32). In addition, the Serrate-binding function of these repeats also appears to have been conserved in the corresponding two EGF repeats of Xenopus Notch (#33ΔCla+XEGF(10-13)). These results unambiguously show that Notch interacts with both Delta and Serrate, and that the same two EGF repeats of Notch mediate both interactions. The Serrate region which is essential for the Notch/Serrate aggregation was also defined. Deleting nucleotides 676-1287 (i.e. amino acids 79-282) (See FIG. 5; SEQ ID NO:3 and NO:4) eliminates the ability of the Serrate protein to aggregate with Notch.

Notch and Serrate appear to aggregate less efficiently than Notch and Delta, perhaps because the Notch-Serrate interaction is weaker. One trivial explanation for this reduced amount of aggregation could be that the Serrate construct simply did not express as much protein at the cell surface as the Delta construct, thereby diminishing the strength of the interaction. Alternatively, the difference in strength of interaction may indicate a fundamental functional difference between Notch-Delta and Notch-Serrate interactions that may be significant in vivo.

### 9. The Cloning, Sequencing, and Expression of Human Notch

### 9.1. Isolation and Sequencing of Human Notch

Clones for the human Notch sequence were originally obtained using the polymerase chain reaction (PCR) to amplify DNA from a 17-18 week human fetal brain cDNA library in the Lambda Zap II vector (Stratagene).

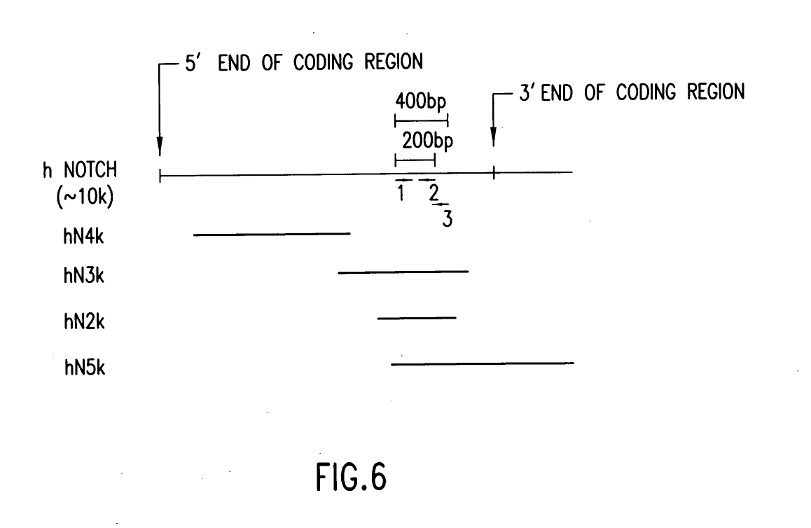

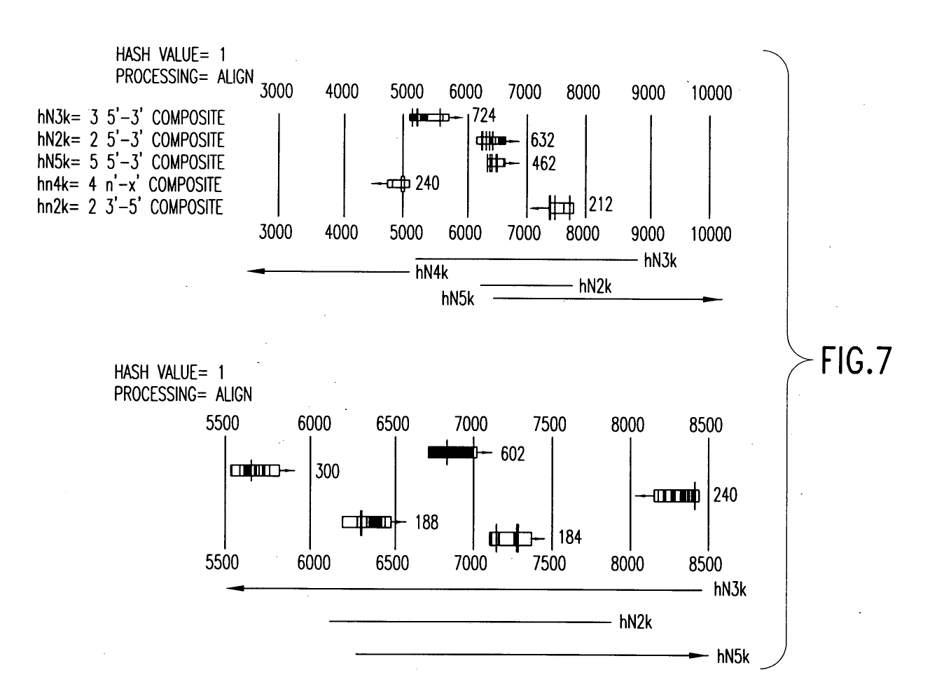

The 400 bp fragment obtained in this manner was then used as a probe with which to screen the same library for human Notch clones. The original screen yielded three unique clones, hN3k, hN2K, and hN5k, all of which were shown by subsequent sequence analysis to fall in the 3′ end of human Notch (FIG. 6). A second screen using the 5′ end of hN3k as probe was undertaken to search for clones encompassing the 5′ end of human Notch. One unique clone, hN4k, was obtained from this screen, and preliminary sequencing data indicate that it contains most of the 5′ end of the gene (FIG. 6). Together, clones hN4k, hN3k and hN5k encompass about 10 kb of the human Notch homolog(s), beginning early in the EGF-repeats and extending into the 3′ untranslated region of the gene. All three clones are cDNA inserts in the EcoRI site of pBluescript SK⁻ (Stratagene). The host E. coli strain is XL1-Blue (see Maniatis, T., 1990, Molecular Cloning, A Laboratory Manual, 2d ed., Cold Spring Harbor Laboratory, Cold Spring Harbor, N.Y., p. A12). An alignment of the human Notch sequences with Drosophila Notch is shown in FIG. 7.

The sequence of various portions of Notch contained in the cDNA clones was determined (by use of Sequenase, U.S. Biochemical Corp.) and is shown for hN2k and hN4k in FIGS. 8 (SEQ ID NO:5-7) and 9 (SEQ ID NO:8, 9), respectively. Further sequence analysis of hN2k revealed that it encodes a human Notch sequence overlapping that contained in hN5k.

The complete nucleotide sequences of the human Notch cDNA contained in hN3k and hN5k was determined by the dideoxy chain termination method using the Sequenase® kit (U.S. Biochemical Corp.). Those nucleotide sequences encoding human Notch, in the appropriate reading frame, were readily identified since there are no introns and translation in only one out of the three possible reading frames yields a sequence which, upon comparison with the published Drosophila Notch deduced amino acid sequence, yields a sequence with a substantial degree of homology to the Drosophila Notch sequence. The DNA and deduced protein sequences of the human Notch cDNA in hN3k and hN5k are presented in FIGS. 10 (SEQ ID NO:10, 11) and 11 (SEQ ID NO:12, 13), respectively. Clone hN3k encodes a portion of a Notch polypeptide starting at approximately the third Notch/lin-12 repeat to several amino acids short of the carboxy-terminal amino acid. Clone hN5k encodes a portion of a Notch polypeptide starting approximately before the cdc10 region through the end of the polypeptide, and also contains a 3′ untranslated region.

Comparing the DNA and protein sequences presented in FIG. 10 (SEQ ID NO:10, 11) with those in FIG. 11 (SEQ ID NO:12, 13) reveals significant differences between the sequences, suggesting that hN3k and hN5k represent part of two distinct Notch-homologous genes. The data thus suggest that the human genome harbors more than one Notch-homologous gene. This is unlike Drosophila, where Notch appears to be a single-copy gene.

Comparison of the DNA and amino acid sequences of the human Notch homologs contained in hN3k and hN5k with the corresponding Drosophila Notch sequences (as published in Wharton et al., 1985, Cell 43:567-581) and with the corresponding Xenopus Notch sequences (as published in Coffman et al., 1990, Science 249:1438-1441 or available from Genbank® (accession number M33874)) also revealed differences.

The amino acid sequence shown in FIG. 10 (hN3k) was compared with the predicted sequence of the TAN-1 polypeptide shown in FIG. 2 of Ellisen et al., August 1991, Cell 66:649-661. Some differences were found between the deduced amino acid sequences; however, overall the hN3k Notch polypeptide sequence is 99% identical to the corresponding TAN-1 region (TAN-1 amino acids 1455 to 2506). Four differences were noted: in the region between the third Notch/lin-12 repeat and the first cdc10 motif, there is an arginine (hN3k) instead of an X (TAN-1 amino acid 1763); (2) there is a proline (hN3k) instead of an X (TAN-1, amino acid 1787); (3) there is a conservative change of an aspartic acid residue (hN3k) instead of a glutamic acid residue (TAN-1, amino acid 2495); and (4) the carboxyl-terminal region differs substantially between TAN-1 amino acids 2507 and 2535.

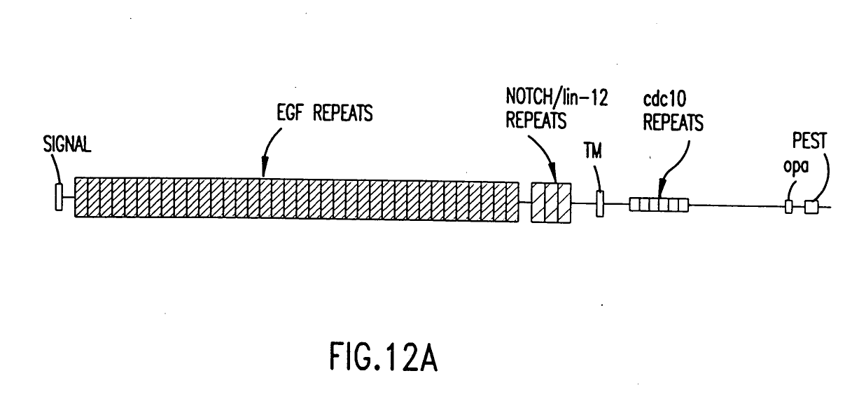

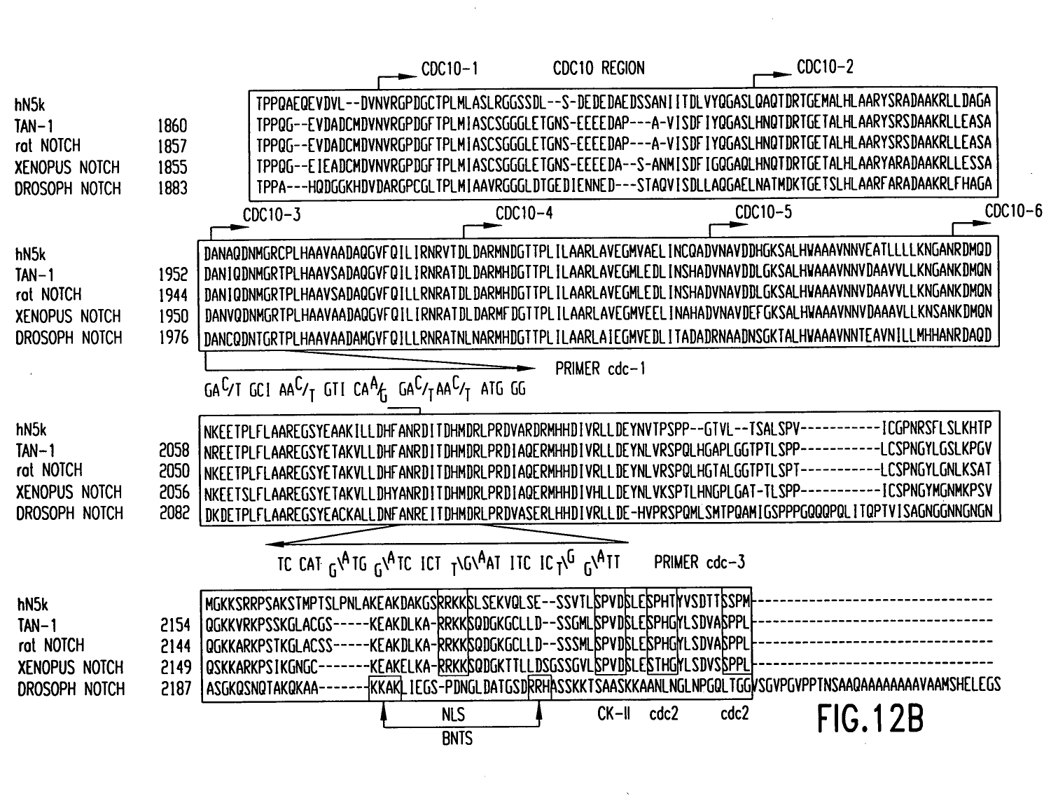

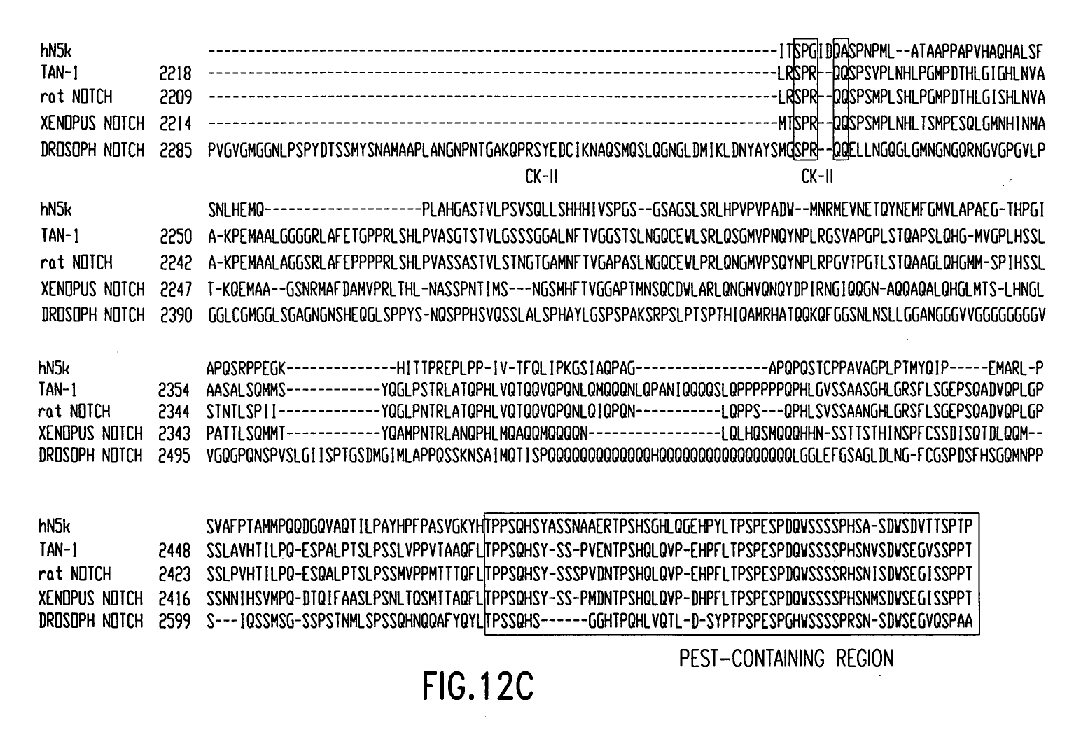

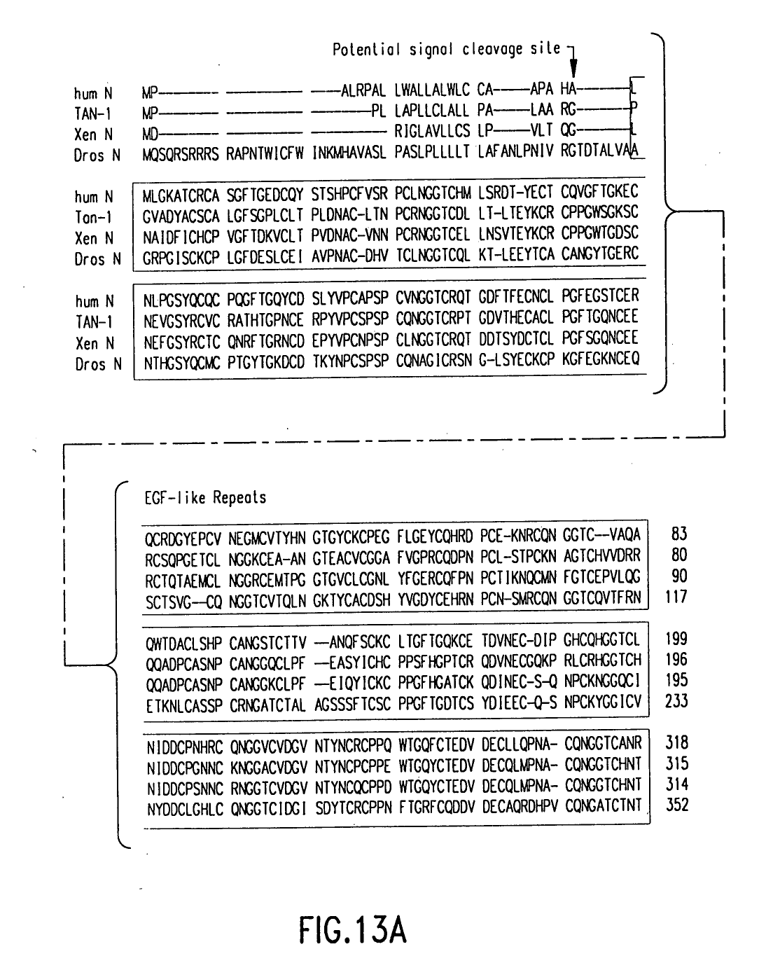

The amino acid sequence shown in FIG. 11 (hN5k) was compared with the predicted sequence of the TAN-1 polypeptide shown in FIG. 2 of Ellisen et al., August 1991, Cell 66:649-661. Differences were found between the deduced amino acid sequences. The deduced Notch polypeptide of hN5k is 79% identical to the TAN-1 polypeptide (64% identical to Drosophila Notch) in the cdc10 region that encompasses both the cc10 motif (TAN-1 amino acids 1860 to 2217) and the well-conserved flanking regions (FIG. 12). The cdc10 region covers amino acids 1860 through 2217 of the TAN-1 sequence. In addition, the hN5k encoded polypeptide is 65% identical to the TAN-1 polypeptide (44% identical to Drosophila Notch) at the carboxy-terminal end of the molecule containing a PEST (proline, glutamic acid, serine, threonine)-rich region (TAN-1 amino acids 2482 to 2551) (FIG. 12B). The stretch of 215 amino acids lying between the aforementioned regions is not well conserved among any of the Notch-homologous clones represented by hN3k, hNSk, and TAN-1. Neither the hN5k polypeptide nor Drosophila Notch shows significant levels of amino acid identity to the other proteins in this region (e.g., hN5k/TAN-1=24% identity; hN5k/Drosophila Notch=11% identity; TAN-1/Drosophila Notch=17% identity). In contrast, Xenopus Notch (Xotch) (SEQ ID NO:16), rat Notch (SEQ ID NO:17), and TAN-1 (SEQ ID NO:18) continue to share significant levels of sequence identity with one another (e.g., TAN-1/rat Notch=75% identity, TAN-1/Xenopus Notch=45% identity, rat Notch/Xenopus Notch=50% identity).

Examination of the sequence of the intracellular domains of the vertebrate Notch homologs shown in FIG. 12B revealed an unexpected finding: all of these proteins, including hN5k, contain a putative CcN motif, associated with nuclear targeting function, in the conserved region following the last of the six cdc10 repeats (FIG. 12B). Although Drosophila Notch lacks such a defined motif, closer inspection of its sequence revealed the presence of a possible bipartite nuclear localization sequence (Robbins et al., 1991, Cell 64:615-623), as well as of possible CK II and cdc2 phosphorylation sites, all in relative proximity to one another, thus possibly defining an alternative type of CcN motif (FIG. 12B).

To isolate clones covering the 5′ end of hN (the human Notch homolog contained in part in hN5k), clone hN2k was used as a probe to screen 260,000 plaques of human fetal brain phage library, commercially available from Stratagene, for crosshybridizing clones. Four clones were identified and isolated using standard procedures (Maniatis et al., 1982, Molecular Cloning: A Laboratory Manual, Cold Spring Harbor Laboratory, Cold Spring Harbor, N.Y.). Four clones were also isolated by hybridization to the Notch-homologous sequence of Adams et al., 1992, Nature 355:632-655, which was obtained from the ATCC.

To isolate clones covering the 5′ end of TAN-1, the human fetal brain library that is commercially available from Stratagene was screened for clones which would extend the sequence to the 5′ end. 880,000 plaques were screened and four clones were identified which crosshybridized with the hN3k sequences. Sequencing confirmed the relative position of these sequences within the Notch protein encoded by TAN-1.

The 5′ sequence of our isolated TAN-1 homolog has been determined through nucleotide number 972 (nucleotide number 1 being the A in the ATG initiation codon), and compared to the sequence as published by Ellisen et al (1991, Cell 66:649-661). At nucleotide 559, our TAN-1 homolog has a G, whereas Ellisen et al. disclose an A, which change results in a different encoded amino acid. Thus, within the first 324 amino acids, our TAN-1-encoded protein differs from that taught by Ellisen et al., since our protein has a Gly at position 187, whereas Ellisen et al. disclose an Arg at that position (as presented in FIG. 13.)

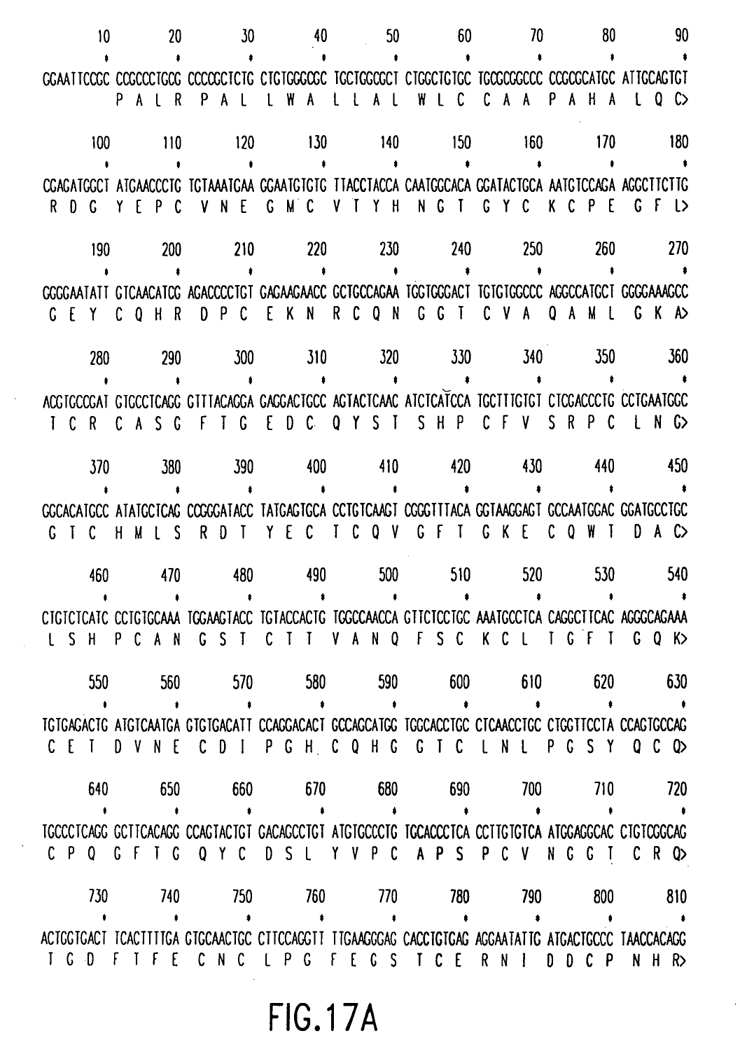

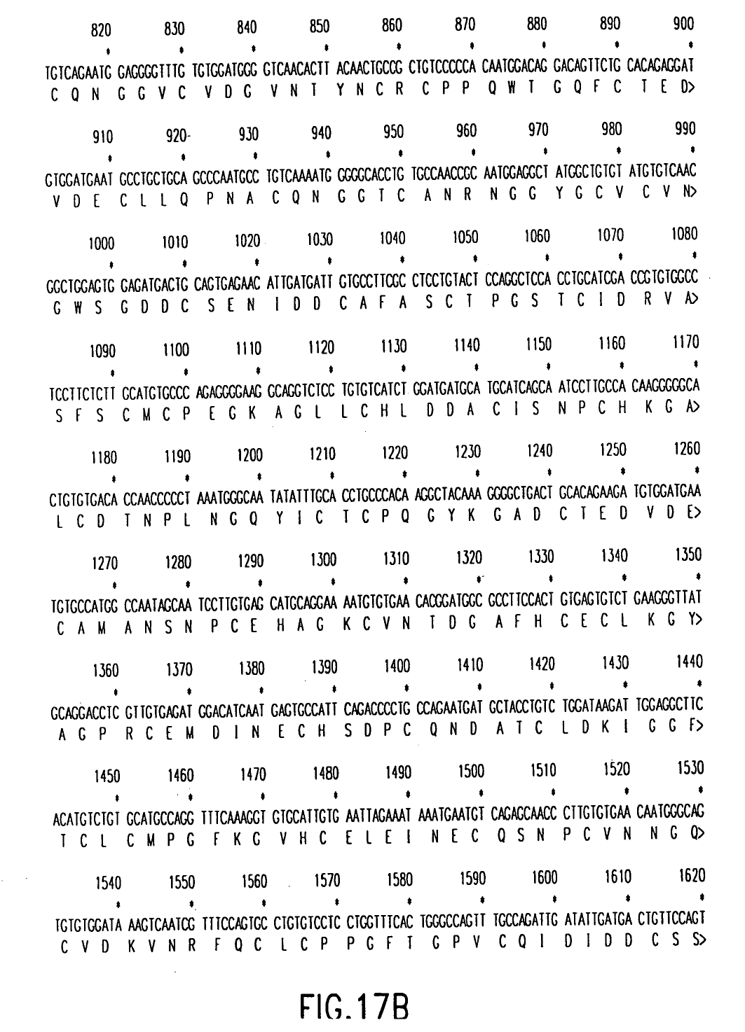

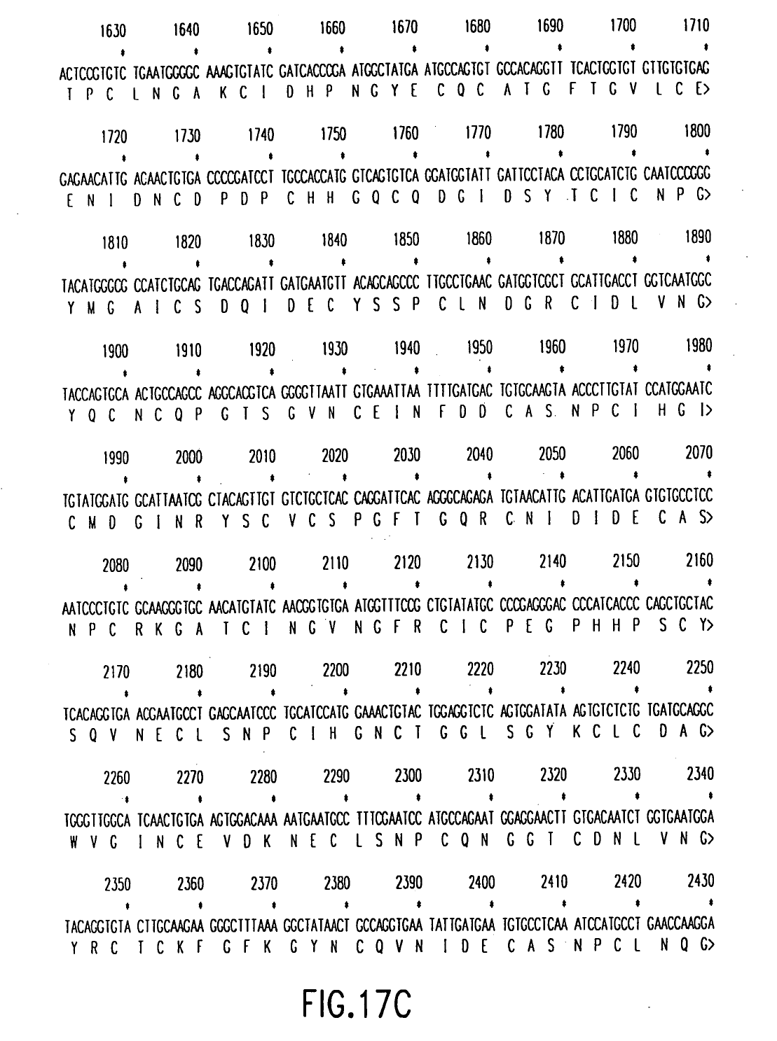

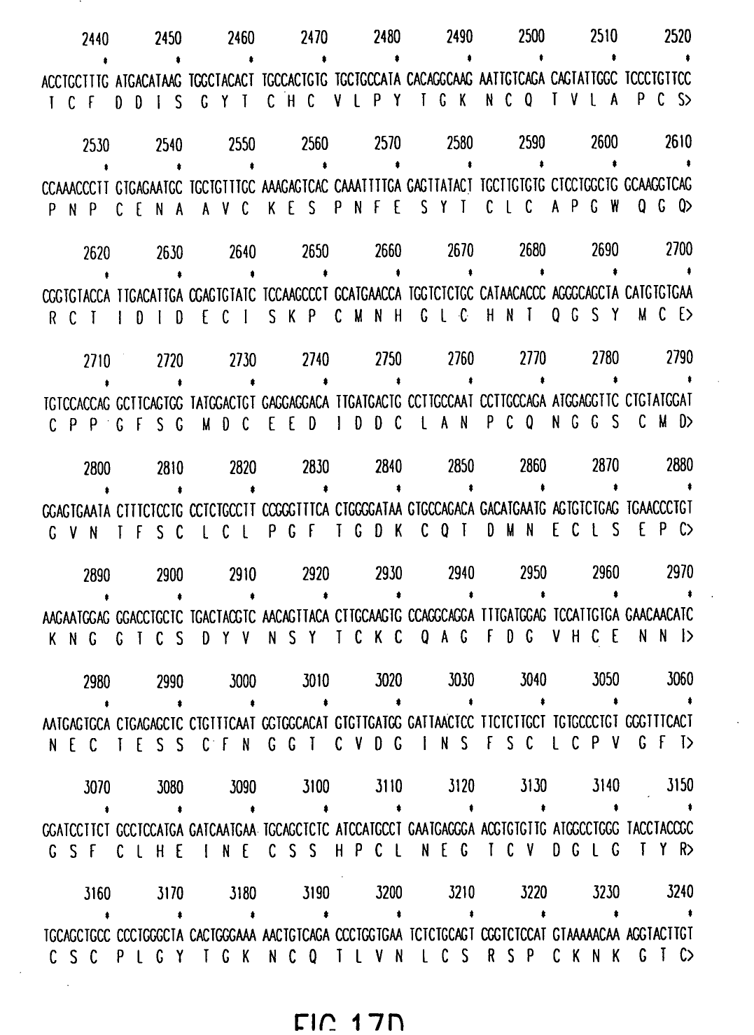

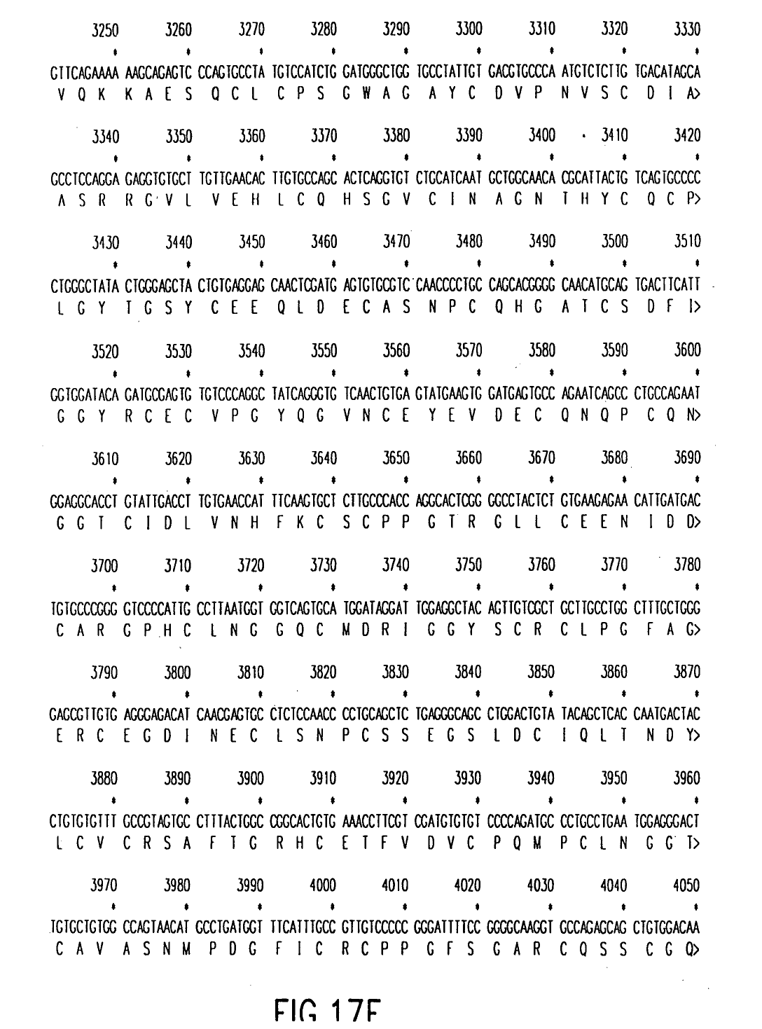

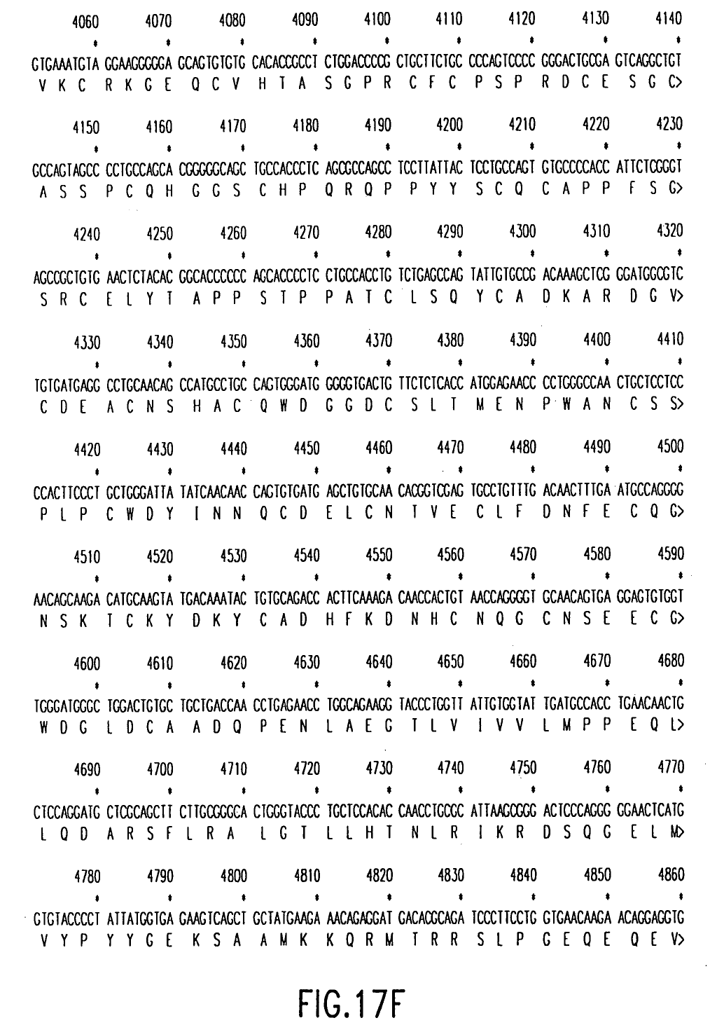

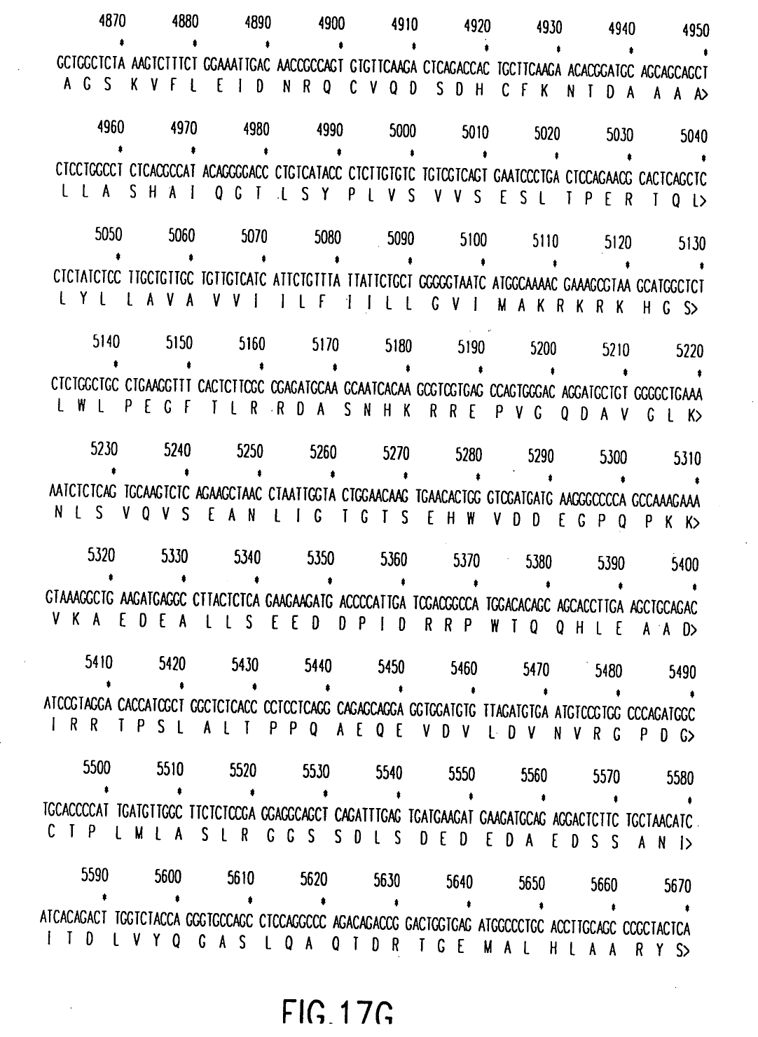

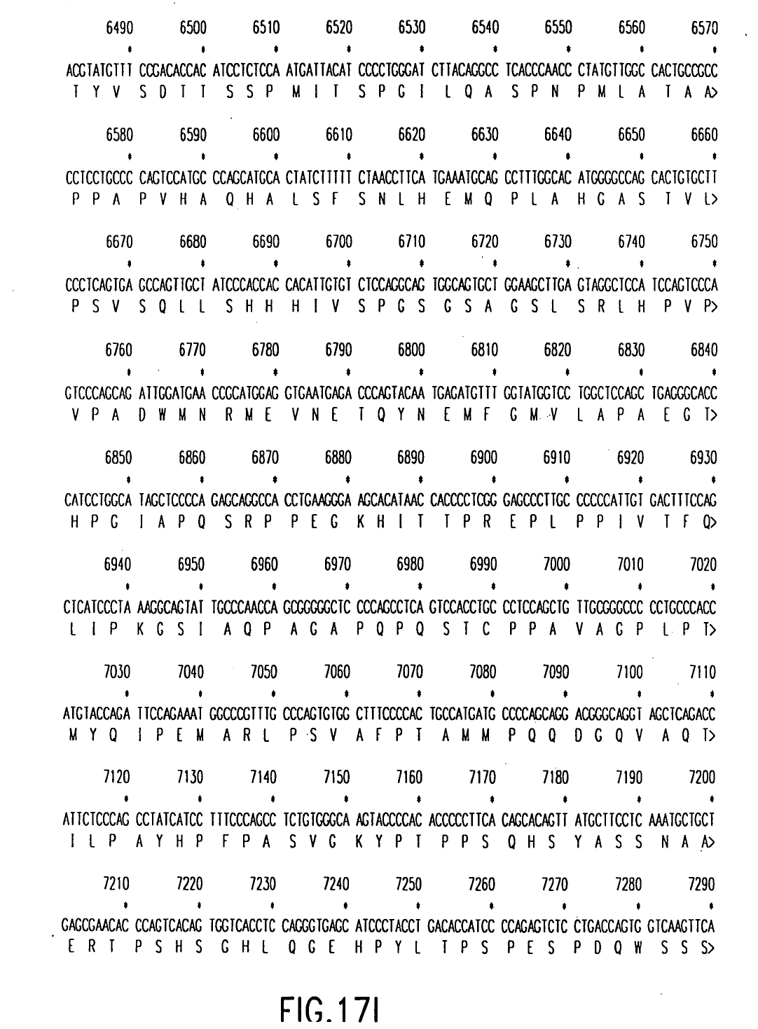

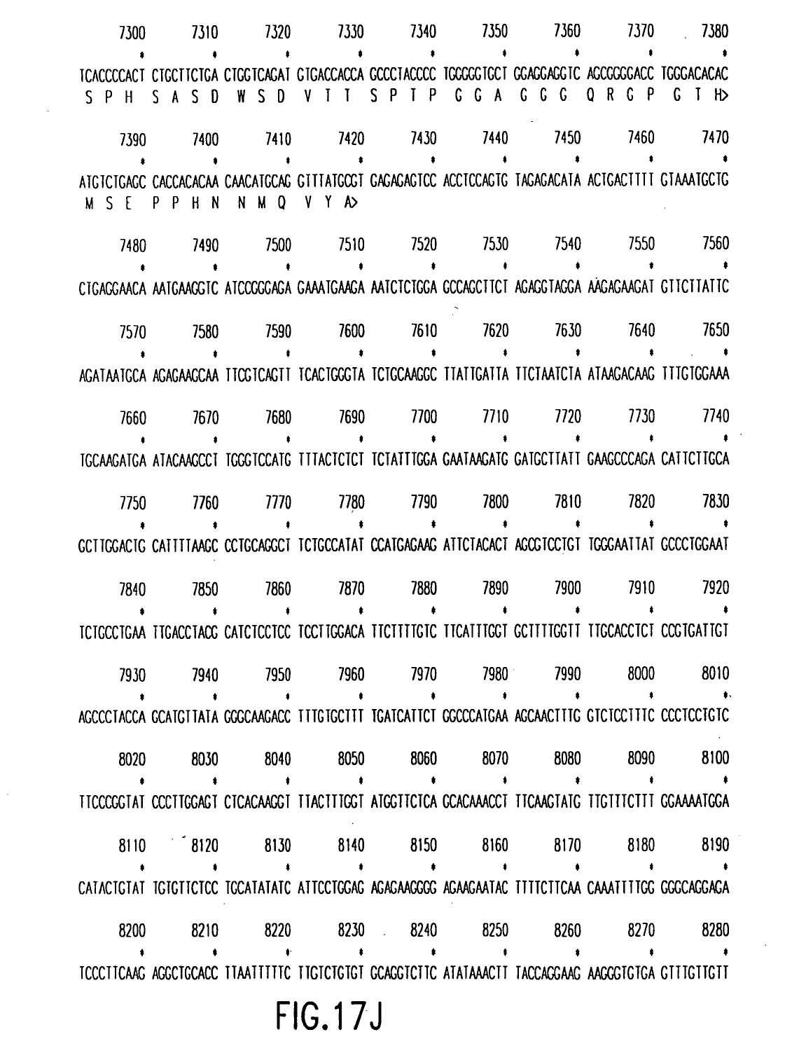

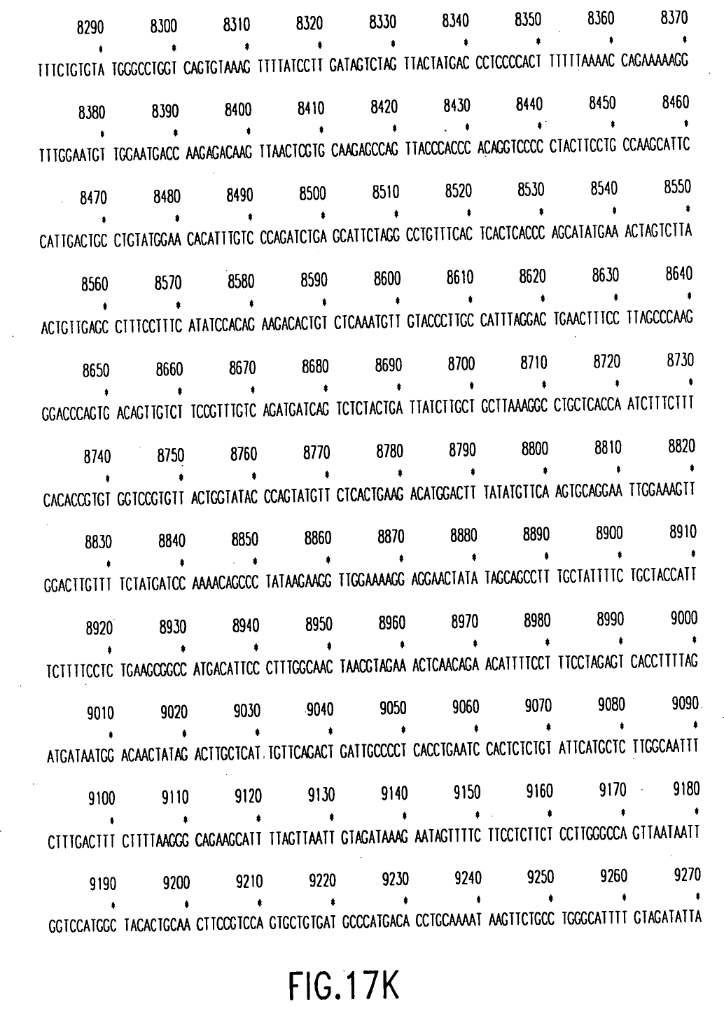

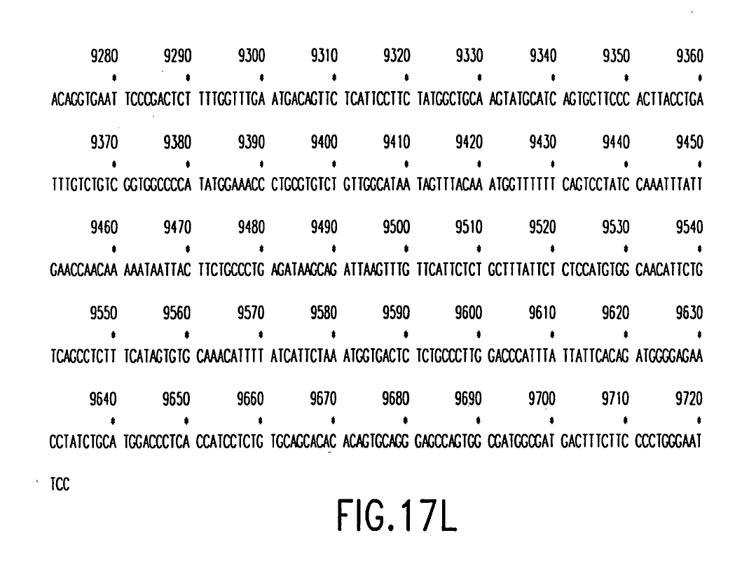

The full-length amino acid sequences of both the hN (SEQ ID NO:19) and TAN-1-encoded (SEQ ID NO:20) proteins, as well as Xenopus and Drosophila Notch proteins, are shown in FIG. 13. The full-length DNA coding sequence (except for that encoding the initiator Met) (contained in SEQ ID NO:21) and encoded amino acid sequence (except that the initiator Met is not shown) (contained in SEQ ID NO:19) of hN are shown in FIG. 17.

### 9.2. Expression of Human Notch

Expression constructs were made using the human Notch cDNA clones discussed in Section 9.1 above. In the cases of hN3k and hN2k, the entire clone was excised from its vector as an EcoRI restriction fragment and subcloned into the EcoRI restriction site of each of the three pGEX vectors (Glutathione S-Transferase expression vectors; Smith and Johnson, 1988, Gene 7, 31-40). This allows for the expression of the Notch protein product from the subclone in the correct reading frame. In the case of hN5k, the clone contains two internal EcoRI restriction sites, producing 2.6, 1.5 and 0.6 kb fragments. Both the 2.6 and the 1.5 kb fragments have also been subcloned into each of the pGEX vectors.

The pGEX vector system was used to obtain expression of human Notch fusion (chimeric) proteins from the constructs described below. The cloned Notch DNA in each case was inserted, in phase, into the appropriate pGEX vector. Each construct was then electroporated into bacteria (E. coli), and was expressed as a fusion protein containing the Notch protein sequences fused to the carboxyl terminus of glutathione S-transferase protein. Expression of the fusion proteins was confirmed by analysis of bacterial protein extracts by polyacrylamide gel electrophoresis, comparing protein extracts obtained from bacteria containing the pGEX plasmids with and without the inserted Notch DNA. The fusion proteins were soluble in aqueous solution, and were purified from bacterial lysates by affinity chromatography using glutathione-coated agarose (since the carboxyl terminus of glutathione S-transferase binds to glutathionine). The expressed fusion proteins were bound by an antibody to Drosophila Notch, as assayed by Western blotting.

The constructs used to make human Notch-glutathione S-transferase fusion proteins were as follows:
hNFP#2—PCR was used to obtain a fragment starting just before the cdc10 repeats at nucleotide 192 of the hN5k insert to just before the PEST-rich region at nucleotide 1694. The DNA was then digested with BamHI and SmaI and the resulting fragment was ligated into pGEX-3. After expression, the fusion protein was purified by binding to glutathione agarose. The purified polypeptide was quantitated on a 4-15% gradient polyacrylamide gel. The resulting fusion protein had an approximate molecular weight of 83 kD.
hN3FP#1—The entire hN3k DNA insert (nucleotide 1 to 3235) was excised from the Bluescript (SK) vector by digesting with EcoRI. The DNA was ligated into pGEX-3.
hN3FP#2—A 3′ segment of hN3k DNA (nucleotide 1847 to 3235) plus some of the polylinker was cut out of the Bluescript (SK) vector by digesting with XmaI. The fragment was ligated into pGEX-1.

Following purification, these fusion proteins are used to make either polyclonal and/or monoclonal antibodies to human Notch.

### 10. Notch Expression in Normal and Malignant Cells

Various human patient tissue samples and cell lines, representing both normal and a wide variety of malignant cells are assayed to detect and/or quantitate expression of Notch. Patient tissue samples are obtained from the pathology department at the Yale University School of Medicine.

The following assays are used to measure Notch expression in patient tissue samples: (a) Northern hybridization; (b) Western blots; (c) in situ hybridization; and (d) immunocytochemistry. Assays are carried out using standard techniques. Northern hybridization and in situ hybridization are carried out (i) using a DNA probe specific to the Notch sequence of clone hN3k; and (ii) using a DNA probe specific to the Notch sequence of clone hN5k. Western blots and immunocytochemistry are carried out using an antibody to Drosophila Notch protein (which also recognizes human Notch proteins).

Northern hybridization and Western blots, as described above, are also used to analyze numerous human cell lines, representing various normal or cancerous tissues. The cell lines tested are listed in Table 2.

TABLE 2
HUMAN CELL LINES

| Tissue/Tumor | Cell line |
|---|---|
| Bone marrow | IM-9 |
| | KG-1 |
| Brain | A-172 |
| | HS 683 |
| | U-87MG |
| | TE 671 |
| Breast | BT-20 |
| | Hs 578Bs |
| | MDA-MB-330 |
| Colon | Caco-2 |
| | SW 48 |
| | T84 |
| | WiDr |
| Embryo | FHs 173We |
| Kidney | A-498 |
| | A-704 |
| | Caki-2 |
| Leukemia | ARH-77 |
| | KG-1 |
| Liver | Hep G2 |
| | WRL 68 |
| Lung | Calu-1 |
| | HLF-a |
| | SK-Lu-1 |
| Lymphoblasts | CCRF-CEM |
| | HuT 78 |
| Lymphoma | Hs 445 |
| | MS116 |
| | U-937 |
| Melanoma | A-375 |
| | G-361 |
| | Hs 294T |
| | SK-MEL-1 |
| Myeloma | IM-9 |
| | RPMI 8226 |
| Neuroblastoma | IMR-32 |
| | SK-N-SH |
| | SK-N-MC |
| Ovary | Caov-3 |
| | Caov-4 |
| | PA-1 |
| Plasma Cells | ARH-77 |
| Sarcoma | A-204 |
| | A673 |
| | HOS |
| Skin | Amdur II |
| | BUD-8 |
| Testis | Tera-1 |
| | Tera-2 |
| Thymus | Hs67 |
| Uterus | AN3 Ca |
| | HEC-1-A |

Malignancies of malignant cell tissue types which are thus shown to specifically express Notch can be treated as described in Section 5.1 et seq.

### 10.1. Expression of Human Notch Protein is Increased in Various Malignancies

As described below, we have found that human Notch protein expression is increased in at least three human cancers, namely cervical, breast, and colon cancer. Immunocytochemical staining of tissue samples from cervical, breast, and colon cancers of human patients showed clearly that the malignant tissue expresses high levels of Notch, at increased levels relative to non-malignant tissue sections. This broad spectrum of different neoplasias in which there is elevated Notch expression suggests that many more cancerous conditions will be seen to upregulate Notch.

Slides of human tumor samples (for breast, colon, and cervical tumors) were obtained from the tissue bank of the Pathology Department, Yale Medical School. The stainings were done using monoclonal antibodies raised against the P1 and P4 fusion proteins which were generated from sequences of hN and TAN-1, respectively.

The P1 and P4 fusion proteins were obtained by insertion of the desired human Notch sequence into the appropriate pGEX expression vector (Smith and Johnson, 1988, Gene 7:31-40; AMRAD Corp., Melbourne, Australia) and were affinity-purified according to the instructions of the manufacturer (AMRAD Corp.). For production of the P1 fusion protein, pGEX-2 was cut with BamHI and ligated to a concatamer which consists of three copies of a 518 bp BamHI-BgIII fragment of hN. Rats were immunized with the expressed protein and monoclonal antibodies were produced by standard procedures. For production of the P4 fusion protein, pGEX-2 was cut with BamHI and ligated to a concatamer which consists of three copies of a 473 bp BamHI-BgIII fragment of TAN-1. Rats were immunized with the expressed protein, and monoclonal antibodies were produced by standard procedures.

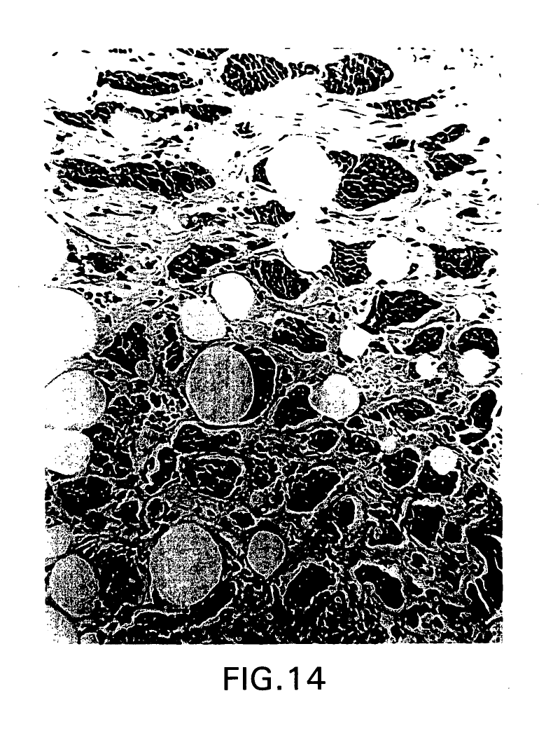

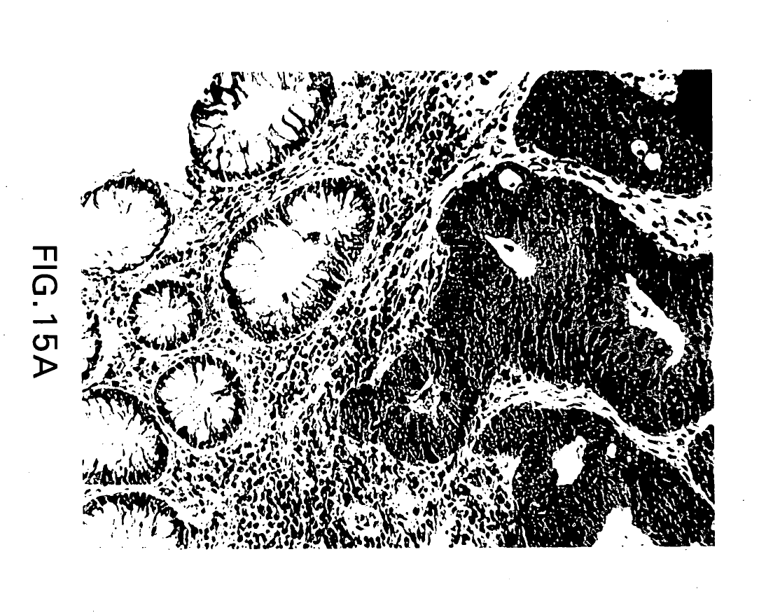

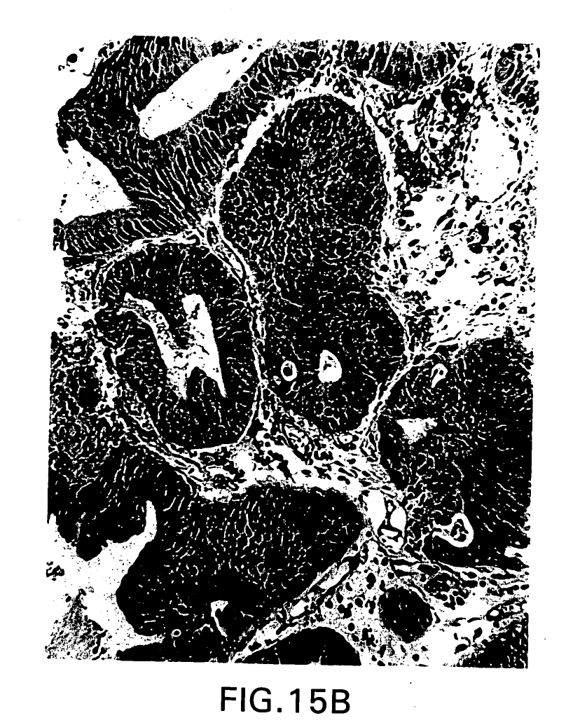

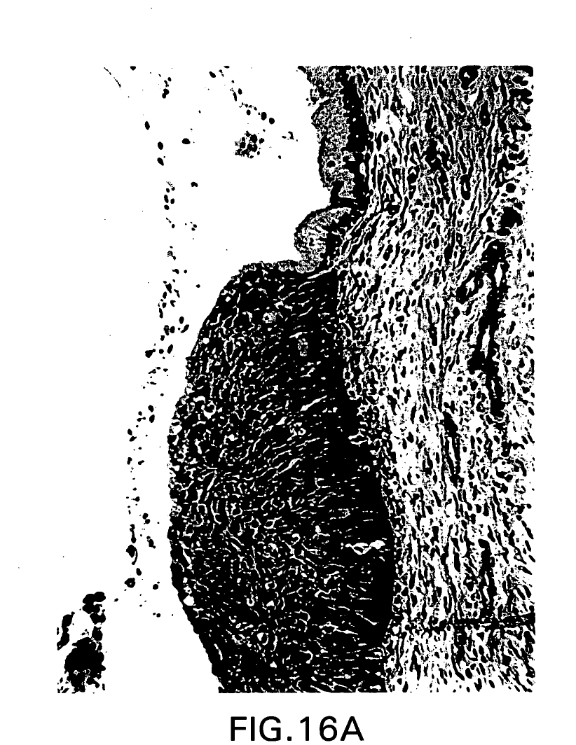

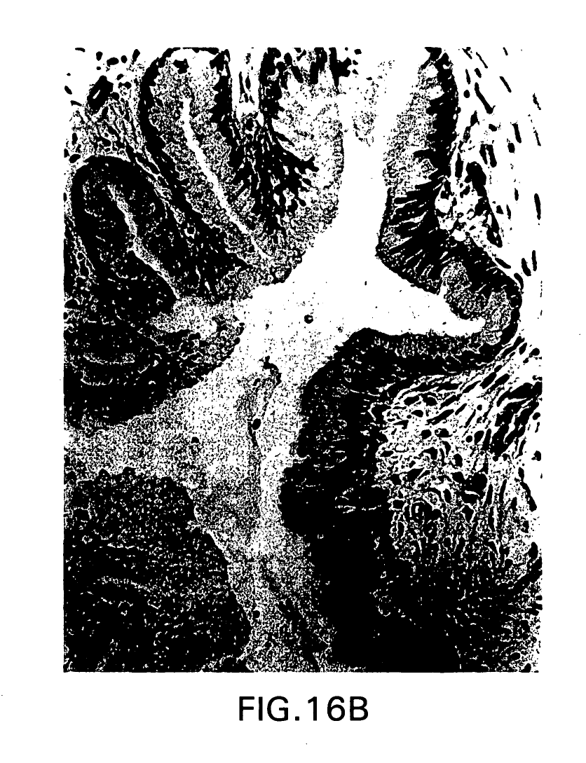

In all tumors examined, the Notch proteins encoded by both human Notch homologs TAN-1 and hN were present at increased levels in the malignant part of the tissue compared to the normal part. Representative stainings are shown in the pictures provided (FIGS. 14-16).

The staining procedure was as follows: The tissues were fixed in paraformaldehyde, embedded in paraffin, cut in 5 micrometer thick sections and placed on glass slides. Then the following steps were carried out:
1. Deparafinization through 4 changes of xylene, 4 minutes each.
2. Removal of xylene through 3 changes in absolute ethanol, 4 minutes each.
3. Gradual rehydration of the tissues by immersing the slides into 95%, 90%, 80%, 60% and 30% ethanol, 4 minutes each. At the end the slides were rinsed in distilled water for 5 minutes.
4. Quenching of endogenous, peroxidase by incubating for 30 minutes in 0.3% hydrogen peroxide in methanol.
5. Washing in PBS (10 mM sodium phosphate pH 7.5, 0.9% NaCl) for 20 minutes.
6. Incubation for 1 hour in blocking solution. (Blocking solution: PBS containing 4% normal rabbit serum and 0.1 Triton X-100.)
7. Incubation overnight at 4° C. with primary antibody diluted in blocking solution. Final concentration of primary antibody 20-50 μg/ml.
8. Washing for 20 minutes with PBS+0.1% Triton X-100 (3 changes).
9. Incubation for 30 minutes with biotinylated rabbit anti-rat antibody: 50 μl of biotinylated antibody (VECTOR) in 10 ml of blocking solution.
10. Washing for 20 minutes with PBS+0.1% Triton X-100 (3 changes).
11. Incubation with ABC reagent (VECTOR) for 30 minutes (the reagent is made in PBS+0.1% Triton X-100).
12. Washing for 20 minutes in PBS+0.1% Triton X-100. Followed by incubation for 2 minutes in PBS+0.5% Triton X-100.
13. Incubation for 2-5 minutes in peroxidase substrate solution. Peroxidase substrate solution: Equal volumes of 0.02% hydrogen peroxide in distilled water and 0.1% diaminobenzidine tetrahydrochloride (DAB) in 0.1 M Tris buffer pH 7.5 are mixed just before the incubation with the tissues. Triton X-100 is added to the final solution at a concentration of 0.5%.
14. Washing for 15 minutes in tap water.
15. Counterstaining for 10 minutes with Mayer's hematoxylin.
16. Washing for 15 minutes in tap water.
17. Dehydration through changes in 30%, 60%, 80%, 90%, 95% and absolute ethanol (4 minutes each).
18. Immersion into xylene (2 changes, 4 minutes each).
19. Mounting, light microscopy.

### 11. Deposit of Microorganisms

The following recombinant bacteria, each carrying a plasmid encoding a portion of human Notch, were deposited on May 2, 1991 with the American Type Culture Collection, 1201 Parklawn Drive, Rockville, Md. 20852, under the provisions of the Budapest Treaty on the International Recognition of the Deposit of Microorganisms for the Purposes of Patent Procedures.

| | | | ATCC |
|---|---|---|---|
| Bacteria | carrying | Plasmid | Accession No. |
| E. coli XL1-Blue | | hN4k | 68610 |
| E. coli XL1-Blue | | hN3k | 68609 |
| E. coli XL1-Blue | | hN5k | 68611 |

The present invention is not to be limited in scope by the microorganisms deposited or the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

1. A pharmaceutical composition comprising a therapeutically effective amount of a Notch protein; and a pharmaceutically acceptable carrier.

2. The composition of claim 1 in which the Notch protein is a human Notch protein.

3-18. (canceled)

19. A pharmaceutical composition comprising a therapeutically effective amount of a protein, said protein comprising a derivative or analog of a Delta protein, which derivative or analog is characterized by the ability in vitro, when expressed on the surface of a first cell, to bind to a Notch protein expressed on the surface of a second cell; and a pharmaceutically acceptable carrier.

20. (canceled)

21. A pharmaceutical composition comprising a therapeutically effective amount of a protein, said protein comprising a derivative or analog of a Serrate protein, which derivative or analog is characterized by the ability in vitro, when expressed on the surface of a first cell, to bind to a Notch protein expressed on the surface of a second cell; and a pharmaceutically acceptable carrier.

22. (canceled)

23. A pharmaceutical composition comprising a therapeutically effective amount of a nucleic acid encoding a Notch protein; and a pharmaceutically acceptable carrier.

24-28. (canceled)

29. A pharmaceutical composition comprising a therapeutically effective amount of a nucleic acid encoding a fragment of a Delta protein, which fragment is characterized by the ability in vitro, when expressed on the surface of a first cell, to bind to a Notch protein expressed on the surface of a second cell; and a pharmaceutically acceptable carrier.

30. A pharmaceutical composition comprising a therapeutically effective amount of a nucleic acid encoding a fragment of a Serrate protein, which fragment is characterized by the ability in vitro, when expressed on the surface of a first cell, to bind to a Notch protein expressed on the surface of a second cell; and a pharmaceutically acceptable carrier.

31. (canceled)

32. A pharmaceutical composition comprising a therapeutically effective amount of an antibody which binds to a Notch protein; and a pharmaceutically acceptable carrier.

33. A pharmaceutical composition comprising a therapeutically effective amount of a fragment or derivative of an antibody to a Notch protein containing the idiotype of the antibody; and a pharmaceutically acceptable carrier.

34. A method of treating a disease or disorder in a subject comprising administering to a subject in need of such treatment a therapeutically effective amount of a molecule which antagonizes the function of a Notch protein.

35-45. (canceled)

46. A method of treating a disease or disorder in a subject comprising administering to a subject in need of such treatment a therapeutically effective amount of a molecule which promotes the function of a Notch protein.

47-89. (canceled)


SEQUENCE LISTING (21 sequences)

SEQ ID NO: 1 - DNA, 2892 bp, Drosophila
  CDS (142)...(2640): Drosophila Delta cDNA (C11)
gaattcggaggaattattcaaaacataaacacaataaacaatttgagtagttgccgcacacacacacacacacagcccgtggattattacactaaaagcgacactcaatccaaaaaatcagcaacaaaaacatcaataaacatgcattggattaaatgtttattaacagcattcatttgcttcacagtcatcgtgcaggttcacagttccggcagctttgagttgcgcctgaagtacttcagcaacgatcacgggcgggacaacgagggtcgctgctgcagcggggagtcggacggagcgacgggcaagtgcctgggcagctgcaagacgcggtttcgcgtctgcctaaagcactaccaggccaccatcgacaccacctcccagtgcacctacggggacgtgatcacgcccattctcggcgagaactcggtcaatctgaccgacgcccagcgcttccagaacaagggcttcacgaatcccatccagttccccttctcgttctcatggccgggtaccttctcgctgatcgtcgaggcctggcatgatacgaacaatagcggcaatgcgcgaaccaacaagctcctcatccagcgactcttggtgcagcaggtactggaggtgtcctccgaatggaagacgaacaagtcggaatcgcagtacacgtcgctggagtacgatttccgtgtcacctgcgatctcaactactacggatccggctgtgccaagttctgccggccccgcgacgattcatttggacactcgacttgctcggagacgggcgaaattatctgtttgaccggatggcagggcgattactgtcacatacccaaatgcgccaaaggctgtgaacatggacattgcgacaaacccaatcaatgcgtttgccaactgggctggaagggagccttgtgcaacgagtgcgttctggaaccgaactgcatccatggcacctgcaacaaaccctggacttgcatctgcaacgagggttggggaggcttgtactgcaaccaggatctgaactactgcaccaaccacagaccctgcaagaatggcggaacctgcttcaacaccggcgagggattgtacacatgcaaatgcgctccaggatacagtggtgatgattgcgaaaatgagatctactcctgcgatgccgatgtcaatccctgccagaatggtggtacctgcatcgatgagccgcacacaaaaaccggctacaagtgtcattgcgccaacggctggagcggaaagatgtgcgaggagaaagtgctcacgtgttcggacaaaccctgtcatcagggaatctgccgcaacgttcgtcctggcttgggaagcaagggtcagggctaccagtgcgaatgtcccattggctacagcggacccaactgcgatctccagctggacaactgcagtccgaatccatgcataaacggtggaagctgtcagccgagcggaaagtgtatttgcccagcgggattttcgggaacgagatgcgagaccaacattgacgattgtcttggccaccagtgcgagaacggaggcacctgcatagatatggtcaaccaatatcgctgccaatgcgttcccggtttccatggcacccactgtagtagcaaagttgacttgtgcctcatcagaccgtgtgccaatggaggaacctgcttgaatctcaacaacgattaccagtgcacctgtcgtgcgggatttactggcaaggattgctctgtggacatcgatgagtgcagcagtggaccctgtcataacggcggcacttgcatgaaccgcgtcaattcgttcgaatgcgtgtgtgccaatggtttcaggggcaagcagtgcgatgaggagtcctacgattcggtgaccttcgatgcccaccaatatggagcgaccacacaagcgagagccgatggtttgaccaatgcccaggtagtcctaattgctgttttctccgttgcgatgcctttggtggcggttattgcggcgtgcgtggtcttctgcatgaagcgcaagcgtaagcgtgctcaggaaaaggacgacgcggaggccaggaagcagaacgaacagaatgcggtggccacaatgcatcacaatggcagtggggtgggtgtagctttggcttcagcctctctgggcggcaaaactggcagcaacagcggtctcaccttcgatggcggcaacccgaatatcatcaaaaacacctgggacaagtcggtcaacaacatttgtgcctcagcagcagcagcggcggcggcggcagcagcggcggacgagtgtctcatgtacggcggatatgtggcctcggtggcggataacaacaatgccaactcagacttttgtgtggctccgctacaaagagccaagtcgcaaaagcaactcaacaccgatcccacgctcatgcaccgcggttcgccggcaggcagctcagccaagggagcgtctggcggaggaccgggagcggcggagggcaagaggatctctgttttaggcgagggttcctactgtagccagcgttggccctcgttggcggcggcgggagtggccggagcctgttcatcccagctaatggctgcagcttcggcagcgggcagcggagcggggacggcgcaacagcagcgatccgtggtctgcggcactccgcatatgtaactccaaaaatccggaagggctcctggtaaatccggagaaatccgcatggaggagctgacagcacatacacaaagaaaagactgggttgggttcaaaatgtgagagagacgccaaaatgttgttgttgattgaagcagtttagtcgtcacgaaaaatgaaaaatctgtaacaggcataactcgtaaactccctaaaaaatttgtatagtaattagcaaagctgtgacccagccgtttcgatcccgaattc

SEQ ID NO: 2 - protein, 833 aa, Drosophila
  Drosophila Delta protein (C11)
MHWIKCLLTAFICFTVIVQVHSSGSFELRLKYFSNDHGRDNEGRCCSGESDGATGKCLGSCKTRFRVCLKHYQATIDTTSQCTYGDVITPILGENSVNLTDAQRFQNKGFTNPIQFPFSFSWPGTFSLIVEAWHDTNNSGNARTNKLLIQRLLVQQVLEVSSEWKTNKSESQYTSLEYDFRVTCDLNYYGSGCAKFCRPRDDSFGHSTCSETGEIICLTGWQGDYCHIPKCAKGCEHGHCDKPNQCVCQLGWKGALCNECVLEPNCIHGTCNKPWTCICNEGWGGLYCNQDLNYCTNHRPCKNGGTCFNTGEGLYTCKCAPGYSGDDCENEIYSCDADVNPCQNGGTCIDEPHTKTGYKCHCANGWSGKMCEEKVLTCSDKPCHQGICRNVRPGLGSKGQGYQCECPIGYSGPNCDLQLDNCSPNPCINGGSCQPSGKCICPAGFSGTRCETNIDDCLGHQCENGGTCIDMVNQYRCQCVPGFHGTHCSSKVDLCLIRPCANGGTCLNLNNDYQCTCRAGFTGKDCSVDIDECSSGPCHNGGTCMNRVNSFECVCANGFRGKQCDEESYDSVTFDAHQYGATTQARADGLTNAQVVLIAVFSVAMPLVAVIAACVVFCMKRKRKRAQEKDDAEARKQNEQNAVATMHHNGSGVGVALASASLGGKTGSNSGLTFDGGNPNIIKNTWDKSVNNICASAAAAAAAAAAADECLMYGGYVASVADNNNANSDFCVAPLQRAKSQKQLNTDPTLMHRGSPAGSSAKGASGGGPGAAEGKRISVLGEGSYCSQRWPSLAAAGVAGACSSQLMAAASAAGSGAGTAQQQRSVVCGTPHM

SEQ ID NO: 3 - DNA, 1320 bp, Drosophila
  CDS (442)...(1320): Drosophila Serrate
ccgagtcgagcgccgtgcttcgagcggtgatgagccccttttctgtcaacgctaaagatctacaaaacatcagcgcctatcaagtggaagtgtcaagtgtgaacaaaacaaaaacgagagaagcacatactaaggtccatataaataataaataataattgtgtgtgataacaacattatccaaacaaaaccaaacaaaacgaaggcaaagtggagaaaatgatacagcatccagagtacggccgttattcagctatccagagcaagtgtagtgtggcaaaatagaaacaaacaaaggcaccaaaatctgcatacatgggctaattaaggctgcccagcgaatttacatttgtgtggtgccaatccagagtgaatccgaaacaaactccatctagatcgccaaccagcatcacgctcgcaaacgcccccagaatgtacaaaatgtttaggaaacattttcggcgaaaaccagctacgtcgtcgtcgttggagtcaacaatagaatcagcagacagcctgggaatgtccaagaagacggcgacaaaaaggcagcgtccgaggcatcgggtacccaaaatcgcgaccctgccatcgacgatccgcgattgtcgatcattaaagtctgcctgcaacttaattgctttaattttaatactgttagtccataagatatccgcagctggtaacttcgagctggaaatattagaaatctcaaataccaacagccatctactcaacggctattgctgcggcatgccagcggaacttagggccaccaagacgataggctgctcgccatgcacgacggcattccggctgtgcctgaaggagtaccagaccacggagcagggtgccagcatatccacgggctgttcgtttggcaacgccaccaccaagatactgggtggctccagctttgtgctcagcgatccgggtgtgggagccattgtgctgccctttacgtttcgttggacgaagtcgtttacgctgatactgcaggcgttggatatgtacaacacatcctatccagatgcggagaggttaattgaggaaacatcatactcgggcgtgatactgccgtcgccggagtggaagacgctggaccacatcgggcggaacgcgcggatcacctaccgtgtccgggtgcaatgcgccgttacctactacaacacgacctgcacgaccttctgccgtccgcgggacgatcagttcggtcactacgcctgcggctccgagggtcagaagctctgcctgaatggctggcagggcgtcaactgcgaggaggccatatgcaaggcgggctgcgaccccgtccacggc

SEQ ID NO: 4 - protein, 293 aa, Drosophila
  Drosophila Serrate protein
MFRKHFRRKPATSSSLESTIESADSLGMSKKTATKRQRPRHRVPKIATLPSTIRDCRSLKSACNLIALILILLVHKISAAGNFELEILEISNTNSHLLNGYCCGMPAELRATKTIGCSPCTTAFRLCLKEYQTTEQGASISTGCSFGNATTKILGGSSFVLSDPGVGAIVLPFTFRWTKSFTLILQALDMYNTSYPDAERLIEETSYSGVILPSPEWKTLDHIGRNARITYRVRVQCAVTYYNTTCTTFCRPRDDQFGHYACGSEGQKLCLNGWQGVNCEEAICKAGCDPVHG

SEQ ID NO: 5 - DNA, 267 bp, Homo sapiens
  Humna Notch (portion)
cggtggacttccttcgtgtattggtgggagccctcgggaacggggggtaacactgaaaggtcgagtacccatttccgtcataacgggttggtcgccccctaggggtcggagtcaggtggacgggaggtcgacaacgcccgggggacgggtggtacatggtgtaaggtctttaccggaccgggcaaacgggtcacaccgaaaggggtgaacggtaactacggggtcgtcctgcccgtccatcgagtctggtaagagggtcgccttaag

SEQ ID NO: 6 - DNA, 574 bp, Homo Sapiens
  misc_feature 399: n = A,T,C or G
gaattccttccattatacgtgacttttctgaaactgtagccaccctagtgtctctaactccctctggagtttgtcagctttggtcttttcaaagagcaggctctcttcaagctccttaatgcgggcatgctccagtttggtctgcgtctcaagatcacctttggtaattgattcttcttcaacccggaactgaaggctggctctcaccctctaggcagagcaggaattccgaggtggatgtgttagatgtgaatgtccgtggcccagatggctgcaccccattgatgttggcttctctccgaggaggcagctcagatttgagtgatgaagatgaagatgcagaggactgttctgctaacatcatcacagacttggtctaccagggtgccagcctccagnccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcacgtgatgccaaggtgtattcagatctgtta

SEQ ID NO: 7 - DNA, 295 bp, Homo Sapiens
  Humna Notch (portion)
tccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggagctataagc

SEQ ID NO: 8 - DNA, 248 bp, Homo Sapiens
  Humna Notch (portion)
gaattccattcaggaggaaagggtggggagagaagcaggcacccactttcccgtggctggactcgttcccaggtggctccaccggcagctgtgaccgccgcaggtgggggcggagtgccattcagaaaattccagaaaagccctaccccaactcggacggcaacgtcacacccgtgggtagcaactggcacacaaacagccagcgtgtctggggcacggggggatggcaccccctgcaggcagagctg

SEQ ID NO: 9 - DNA, 323 bp, Homo Sapiens
  misc_feature 38, 39, 263, 264, 265, 283, 285, 306: n = A,T,C or G
tacgtatctcgagcacagacagctgacgtacacttttnnagtgcgagggacattcgtccgaccagtacgaacatttaggctcagtacggtaggtccatggccaagactaggagacgtagggagctacaggtcccgctcgctaaactcggaccactgaaacctccggtcgacagtcggtaagcgaacaagagggccagatcttagagaaggtgtcgcggcgagactcgggctcgggtcaggcggccttaaggacgtcgggcccnnnaggtgatcaagatctcgncncggcgggcgccacctcgaggncgaaaacaagggaaatc

SEQ ID NO: 10 - DNA, 3234 bp, Homo Sapiens
  CDS (1)...(3234): Human Notch contained in Plasmid cDNA clone hN3k
tgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggatggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgttcctgtaatggtaatgatccgatcttcggatccttctaaaggctcatcaattttgatcgaagctcccgactcatgg

SEQ ID NO: 11 - protein, 1078 aa, Homo Sapiens
  Human Notch contained in Plasmid cDNA clone hN3k
CQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPVPVMVMIRSSDPSKGSSILIEAPDSW

SEQ ID NO: 12 - DNA, 4268 bp, Homo Sapiens
  CDS (2)...(1972): Human Notch contained in cDNA clone of hN5k
ggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggaccctcaccatcctctgtgcagcacacacagtgcagggagccagtggcgatggcgatgactttcttcccctg

SEQ ID NO: 13 - protein, 657 aa, Homo Sapiens
  Human Notch contained in Plasmid cDNA clone hN5k
EVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 14 - protein, 77 aa, Drosophila
  Drosophila Notch
EDIDECDQGSPCEHNGICVNTPGSYRCNCSQGFTGPRCETNINECESHPCQNEGSCLDDPGTFRCVCMPGFTGTQCE

SEQ ID NO: 15 - protein, 78 aa, Xenopus
  Xenopus Notch
NDVDECSLGANPCEHGGRCTNTLGSFQCNCPQGYAGPRCEIDVNECLSNPCQNDSTCLDQIGEFQCICMPGYEGLYCE

SEQ ID NO: 16 - protein, 654 aa, Xenopus
  Xenopus Notch
TPPQGEIEADCMDVNVRGPDGFTPLMIASCSGGGLETGNSEEEEDASANMISDFIGQGAQLHNQTDRTGETALHLAARYARADAAKRLLESSADANVQDNMGRTPLHAAVAADAQGVFQILIRNRATDLDARMFDGTTPLILAARLAVEGMVEELINAHADVNAVDEFGKSALHWAAAVNNVDAAAVLLKNSANKDMQNNKEETSLFLAAREGSYETAKVLLDHYANRDITDHMDRLPRDIAQERMHHDIVHLLDEYNLVKSPTLHNGPLGATTLSPPICSPNGYMGNMKPSVQSKKARKPSIKGNGCKEAKELKARRKKSQDGKTTLLDSGSSGVLSPVDSLESTHGYLSDVSSPPLMTSPFQQSPSMPLNHLTSMPESQLGMNHINMATKQEMAAGSNRMAFDAMVPRLTHLNASSPNTIMSNGSMHFTVGGAPTMNSQCDWLARLQNGMVQNQYDPIRNGIQQGNAQQAQALQHGLMTSLHNGLPATTLSQMMTYQAMPNTRLANQPHLMQAQQMQQQQNLQLHQSMQQQHHNSSTTSTHINSPFCSSDISQTDLQQMSSNNIHSVMPQDTQIFAASLPSNLTQSMTTAQFLTPPSQHSYSSPMDNTPSHQLQVPDHPFLTPSPESPDQWSSSSPHSNMSDWSEGISSPPT

SEQ ID NO: 17 - protein, 666 aa, Rattus
  rat Notch
TPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLAVEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACSSKEAKDLKARRKKSQDGKGCLLDSSSMLSPVDSLESPHGYLSDVASPPLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHGMMSPIHSSLSTNTLSPIIYQGLPNTRLATQPHLVQTQQVQPQNLQIQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSRHSNISDWSEGISSPPT

SEQ ID NO: 18 - protein, 681 aa, Rattus
  rat TAN-1
TPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVENTPSHQLQVPEHPFLTPSPESPDQWSSSSPHSNVSDWSEGVSSPPT

SEQ ID NO: 19 - protein, 2471 aa, Homo Sapiens
  Human Notch protein encoded by the hN homolog (contained in part in plasmid hN5k)
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCNQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 20 - protein, 2556 aa, Homo Sapiens
  Human Notch protein encoded by the TAN-1 homolog
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREELGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVENTPSHQLQVPEHPFLTPSPESPDQWSSSSPHSNVSDWSEGVSSPPTSMQSQIARIPEAFK

SEQ ID NO: 21 - DNA, 9723 bp, Homo Sapiens
  CDS (10)...(7419): Human Notch homolog (hN) entire coding sequence
ggaattccgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgtgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtaaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggaccctcaccatcctctgtgcagcacacacagtgcagggagccagtggcgatggcgatgactttcttcccctgggaattcc